SEQUENCE LISTING (379 sequences)

SEQ ID NO: 1 - DNA, 1581 bp, ARABIDOPSIS THALIANA
atggagagcgacgaagcagcagcagtgtctcctcaagcaacgacaccgagcggaggaaccggagcttctgggccgaagaagagaggtcggaaacctaaaaccaaggaagattctcagacgccgtcgtctcagcaacagagcgatgttaaaatgaaagaaagtgggaagaaaacgcagcagtcgccgagtgttgacgagaagtactctcagtggaaaggtctcgtccccattctctacgactggctcgctaaccataacctcgtctggccttcactctcttgcagatggggtccgcagcttgagcaagcaacctacaagaatcgccagcgtctgtacctctcagagcaaactgatggaagtgtgcccaatactttggtcatagcaaattgtgaagttgttaagccaagggttgctgcagcagagcacatttctcagttcaatgaagaagcacgttctccatttgtgaagaagtacaagaccatcattcaccctggagaggttaacagaatcagggaactcccacagaatagtaagattgttgctactcacaccgacagtcctgattcaaagagcatcctttgttcggacatatgtccagtttcagagttatctaaatacaatatgtccagtttggatgttgaaacccaaccaaaccgtcatgctgtgcttggagctgcaaattcccgtccagatttgatactaactgggcaccaagataatgctgaatttgctcttgccatgtgcccaacggaaccctttgtgctctccggaggcaaggacaagtcagttgttttgtggagtatccaagatcacatcacaacgattgggacagattccaaatcatctggatctatcatcaaacagactggtgaaggtactgataagaatgagagtcctactgttggcccacgaggtgtatatcatggccatgaagatacagttgaagatgtggcattcagcccgacgagtgcacaagaattctgcagtgttggtgatgattcttgccttatactatgggatgcgagaactggcacaaaccctgtcacgaaggttgaaaaagcgcatgatgctgatcttcattgtgttgattggaatcctcatgacgacaatctgatcctgacagggtcagcagacaacactgtccggttgtttgatcgtaggaagcttaccgctaatggagttggttcgcctatttacaaatttgagggacacaaagctgctgttctttgtgttcagtggtctcctgataagtcatccgtctttgggagctctgcagaagatggtctcttgaacatctgggattatgacagggtcagtaagaagtctgatcgtgcagctaaaagccccgctgggctcttcttccagcatgctggtcacagggacaaagttgttgatttccactggaatgcttcagacccttggactattgtcagtgtttctgatgactgtgagactactggtggaggtggaacattgcagatatggcggatgagtgacttgatttacagaccagaagaagaagtcgtggcagaattggagaagttcaagtcgcatgttatgacttgtgcctccaagccttaa

SEQ ID NO: 2 - protein, 526 aa, ARABIDOPSIS THALIANA
MESDEAAAVSPQATTPSGGTGASGPKKRGRKPKTKEDSQTPSSQQQSDVKMKESGKKTQQSPSVDEKYSQWKGLVPILYDWLANHNLVWPSLSCRWGPQLEQATYKNRQRLYLSEQTDGSVPNTLVIANCEVVKPRVAAAEHISQFNEEARSPFVKKYKTIIHPGEVNRIRELPQNSKIVATHTDSPDSKSILCSDICPVSELSKYNMSSLDVETQPNRHAVLGAANSRPDLILTGHQDNAEFALAMCPTEPFVLSGGKDKSVVLWSIQDHITTIGTDSKSSGSIIKQTGEGTDKNESPTVGPRGVYHGHEDTVEDVAFSPTSAQEFCSVGDDSCLILWDARTGTNPVTKVEKAHDADLHCVDWNPHDDNLILTGSADNTVRLFDRRKLTANGVGSPIYKFEGHKAAVLCVQWSPDKSSVFGSSAEDGLLNIWDYDRVSKKSDRAAKSPAGLFFQHAGHRDKVVDFHWNASDPWTIVSVSDDCETTGGGGTLQIWRMSDLIYRPEEEVVAELEKFKSHVMTCASKP

SEQ ID NO: 3 - DNA, 1524 bp, ARABIDOPSIS THALIANA
atggagagcgacgaagcagcagcagtgtctcctcaagcaacgacaccgagcggaggaaccggagcttctgggccgaagaagagaggtcggaaacctaaaaccaaggaagattctcagacgccgtcgtctcagcaacagagcgatgttaaaatgaaagaaagtgggaagaaaacgcagcagtcgccgagtgttgacgagaagtactctcagtggaaaggtctcgtccccattctctacgactggctcgctaaccataacctcgtctggccttcactctcttgcagatggggtccgcagcttgagcaagcaacctacaagaatcgccagcgtctgtacctctcagagcaaactgatggaagtgtgcccaatactttggtcatagcaaattgtgaagttgttaagccaagggttgctgcagcagagcacatttctcagttcaatgaagaagcacgttctccatttgtgaagaagtacaagaccatcattcaccctggagaggttaacagaatcagggaactcccacagaatagtaagattgttgctactcacaccgacagtcctgatgttctcatttgggatgttgaaacccaaccaaaccgtcatgctgtgcttggagctgcaaattcccgtccagatttgatactaactgggcaccaagataatgctgaatttgctcttgccatgtgcccaacggaaccctttgtgctctccggaggcaaggacaagtcagttgttttgtggagtatccaagatcacatcacaacgattgggacagattccaaatcatctggatctatcatcaaacagactggtgaaggtactgataagaatgagagtcctactgttggcccacgaggtgtatatcatggccatgaagatacagttgaagatgtggcattcagcccgacgagtgcacaagaattctgcagtgttggtgatgattcttgccttatactatgggatgcgagaactggcacaaaccctgtcacgaaggttgaaaaagcgcatgatgctgatcttcattgtgttgattggaatcctcatgacgacaatctgatcctgacagggtcagcagacaacactgtccggttgtttgatcgtaggaagcttaccgctaatggagttggttcgcctatttacaaatttgagggacacaaagctgctgttctttgtgttcagtggtctcctgataagtcatccgtctttgggagctctgcagaagatggtctcttgaacatctgggattatgacagggtcagtaagaagtctgatcgtgcagctaaaagccccgctgggctcttcttccagcatgctggtcacagggacaaagttgttgatttccactggaatgcttcagacccttggactattgtcagtgtttctgatgactgtgagactactggtggaggtggaacattgcagatatggcggatgagtgacttgatttacagaccagaagaagaagtcgtggcagaattggagaagttcaagtcgcatgttatgacttgtgcctccaagccttaa

SEQ ID NO: 4 - protein, 507 aa, ARABIDOPSIS THALIANA
MESDEAAAVSPQATTPSGGTGASGPKKRGRKPKTKEDSQTPSSQQQSDVKMKESGKKTQQSPSVDEKYSQWKGLVPILYDWLANHNLVWPSLSCRWGPQLEQATYKNRQRLYLSEQTDGSVPNTLVIANCEVVKPRVAAAEHISQFNEEARSPFVKKYKTIIHPGEVNRIRELPQNSKIVATHTDSPDVLIWDVETQPNRHAVLGAANSRPDLILTGHQDNAEFALAMCPTEPFVLSGGKDKSVVLWSIQDHITTIGTDSKSSGSIIKQTGEGTDKNESPTVGPRGVYHGHEDTVEDVAFSPTSAQEFCSVGDDSCLILWDARTGTNPVTKVEKAHDADLHCVDWNPHDDNLILTGSADNTVRLFDRRKLTANGVGSPIYKFEGHKAAVLCVQWSPDKSSVFGSSAEDGLLNIWDYDRVSKKSDRAAKSPAGLFFQHAGHRDKVVDFHWNASDPWTIVSVSDDCETTGGGGTLQIWRMSDLIYRPEEEVVAELEKFKSHVMTCASKP

SEQ ID NO: 5 - DNA, 3763 bp, ARABIDOPSIS THALIANA
ccgaaaaatcataatattgtctatatgtaaatataaatataaatattttagttatatttatatttaagatatgtttgggtaccagttcgggttcgggtttttcaggttttgaagtttagattcagtcggatatttgaaaatttcaggttcgaatttagattgggatttttggattgggatttttggatcgggttcagatcggttttttcggattcaggtattatgtccaaagtaaaaatggtaaacggtggttgttttgtttgtatttgtaaagcagaagagagagagatatagagacactgaaagcaaagaccaaaaaagaaaaattaaaaagagagagaggaaaatggagagcgacgaagcagcagcagtgtctcctcaagcaacgacaccgagcggaggaaccggagcttctgggccgaagaagagaggtcggaaacctaaaaccaaggaagattctcagacgccgtcgtctcagcaacagagcgatgttaaaatgaaagaaagtgggaagaaaacgcagcagtcgccgagtgttgacgagaagtactctcagtggaaaggtctcgtccccattctctacgactggctcgctaaccataacctcgtctggccttcactctcttgcaggtctccccctttctcctttcctctccttctagggtttcgtttcgtaatcgtttcttagctttgaacattctcatgtttggaatgaatttagtaaaatcttacacatacattttctcgatttctgggtttaagtgagattgttgcgattgttctagttagggttttggatgtggctctgtcttcataccttgatatatctgatgttctattcatgaattgttactattgattaccttgttggttactaatgactaagaggaattttcagtttctctgagtgtttatatctgatgaagtctttagttgttgtgctaagagtttccatttggtgaattgttgtttgatttttttatagatggggtccgcagcttgagcaagcaacctacaagaatcgccagcgtctgtacctctcagagcaagtaagtttttagctttctcttgtatcttgttgtctcatcttctttatatacttctcatcgtattatttgtatttttcttggttgtgtcaccagactgatggaagtgtgcccaatactttggtcatagcaaattgtgaagttgttaagccaagggttgctgcagcagagcacatttctcaggtattatgtggtttaatactaagcttgtgtcctttccatatcctactccacactacaattggtttcatgtttgacacttatatactatcttctgaaaatgtgttctcagttcaatgaagaagcacgttctccatttgtgaagaagtacaagaccatcattcaccctggagaggtgtgaattctgcccactcttgagatatttctgtattgacattgttctttttagttctatttggtttgttaattgtatctgcatccctgtttcatctgtccatgaagttattcgtttggcacgttggtgaaagtaaattttgatgtgtattcattactaatttgcaattgcaggttaacagaatcagggaactcccacagaatagtaagattgttgctactcacaccgacagtcctgatgtgagtgctgcttctattttgttatggtcatagcaacttgaaatatgtcggtttcatatttctgtatttggcagtcaaagagcatcctttgttcggacatatgtccagtttcagagttatctaaatacaatatgtccagtttggatgttgaaacccaaccaaaccgtcatgctgtgcttggagctgcaaattcccgtccagatttggtatgtccacttctgagaatgttgttttatgctttattcttgtttgtttctcatcattggaagtgataaatctctttgatatcttcttaaatagtgcttcttgtttgcatcatctgaatgaaccatttttcatgcagatactaactgggcaccaagataatgctgaatttgctcttgccatgtgcccaacggaaccctttgtgctctccggaggtttgtgtttctgtaatttgtagagtccaatcctgtggtttgccagtttctcatacaaaagttcttctcttaggcaaggacaagtcagttgttttgtggagtatccaagatcacatcacaacgattgggacagattccaaatcatctggatctatcatcaaacagactggtgaaggtactgataagaatgagagtcctactgttggcccacgaggtgtatatcatggccatgaagatacagttgaagatgtggcattcagcccgacgaggtaacttcttagaacagactccttctattgatatcgtgtttgtttatgcatactgcagatattttcatgattttctaataatacttctggtgaacttttataccgtgaagtgcacaagaattctgcagtgttggtgatgattcttgccttatactatgggatgcgagaactggcacaaaccctgtcacgaaggtactctatcttttgaatcctatcaaaagtttgaagatttacctccttttgatattatatcttacttttttgttttccaggttgaaaaagcgcatgatgctgatcttcattgtgttgattggaatcctcatgacgacaatctgatcctgacagggtatggagaaatacatacaaatagatgattaatacatacttagtatctaattaagaaattgatgaatatttcaggtcagcagacaacactgtccggttgtttgatcgtaggaagcttaccgctaatggagttggttcgcctatttacaaatttgagggacacaaagctgctgttctttgtgttcaggtataatcaactttttttttttttttccttctttgtatgaagtatatctcttaacccactgacactatcttgttattcaattcagtggtctcctgataagtcatccgtctttgggagctctgcagaagatggtctcttgaacatctgggattatgacagggtgtgtacatagttcactcagatgtctaaaattaatctttcttcactatcatcactgaaacatattcattgtactcatgtttggtttgtttaattaaccatcaggtcagtaagaagtctgatcgtgcagctaaaagccccgctgggctcttcttccagcatgctggtcacaggttcttaaagacttatcttgatttttcttgattgctttctcattttacttgcttctaagttcccttgtttataaaccatattagggacaaagttgttgatttccactggaatgcttcagacccttggactattgtcagtgtttctgatgactgtgagactactggtggaggtggaacattgcaggtaaccttgaaatctttcttggtaccttgataagcaattttattgacataccgttaaatgttgtttatactttcttctatggcagatatggcggatgagtgacttgatttacagaccagaagaagaagtcgtggcagaattggagaagttcaagtcgcatgttatgacttgtgcctccaagccttaagagtaaagaaaacccattgtctatctatctatcgcctatggtaaactaatgcgggttttagcgaggagtcttggtttttgtaaggctggtttgtcttttgagatattggtggtagcttttaggacctttccatatcagttaggggtacatggttctggttcatgatcctgtttcatcagactcttaggtgctgttttgtt

SEQ ID NO: 6 - DNA, 3776 bp, ARABIDOPSIS THALIANA
ccgaaaaatcataatattgtctatatgtaaatataaatataaatattttagttatatttatatttaagatatgtttgggtaccagttcgggttcgggtttttcaggttttgaagtttagattcagtcggatatttgaaaatttcaggttcgaatttagattgggatttttggattgggatttttggatcgggttcagatcggttttttcggattcaggtattatgtccaaagtaaaaatggtaaacggtggttgttttgtttgtatttgtaaagcagaagagagagagatatagagacactgaaagcaaagaccaaaaaagaaaaattaaaaagagagagaggaaaatggagagcgacgaagcagcagcagtgtctcctcaagcaacgacaccgagcggaggaaccggagcttctgggccgaagaagagaggtcggaaacctaaaaccaaggaagattctcagacgccgtcgtctcagcaacagagcgatgttaaaatgaaagaaagtgggaagaaaacgcagcagtcgccgagtgttgacgagaagtactctcagtggaaaggtctcgtccccattctctacgactggctcgctaaccataacctcgtctggccttcactctcttgcaggtctccccctttctcctttcctctccttctagggtttcgtttcgtaatcgtttcttagctttgaacattctcatgtttggaatgaatttagtaaaatcttacacatacattttctcgatttctgggtttaagtgagattgttgcgattgttctagttagggttttggatgtggctctgtcttcataccttgatatatctgatgttctattcatgaattgttactattgattaccttgttggttactaatgactaagaggaattttcagtttctctgagtgtttatatctgatgaagtctttagttgttgtgctaagagtttccatttggtgaattgttgtttgatttttttatagatggggtccgcagcttgagcaagcaacctacaagaatcgccagcgtctgtacctctcagagcaagtaagtttttagctttctcttgtatcttgttgtctcatcttctttatatacttctcatcgtattatttgtatttttcttggttgtgtcaccagactgatggaagtgtgcccaatactttggtcatagcaaattgtgaagttgttaagccaagggttgctgcagcagagcacatttctcaggtattatgtggtttaatactaagcttgtgtcctttccatatcctactccacactacaattggtttcatgtttgacacttatatactatcttctgaaaatgtgttctcagttcaatgaagaagcacgttctccatttgtgaagaagtacaagaccatcattcaccctggagaggtgtgaattctgcccactcttgagatatttctgtattgacattgttctttttagttctatttggtttgttaattgtatctgcatccctgtttcatctgtccatgaagttattcgtttggcacgttggtgaaagtaaattttgatgtgtattcattactaatttgcaattgcaggttaacagaatcagggaactcccacagaatagtaagattgttgctactcacaccgacagtcctgatgtgagtgctgcttctattttgttatggtcatagcaacttgaaatatgtcggtttcatatttctgtatttggcagtcaaagagcatcctttgttcggacatatgtccagtttcagagttatctaaatacaatatgttgatttcaggttctcatttgggatgttgaaacccaaccaaaccgtcatgctgtgcttggagctgcaaattcccgtccagatttggtatgtccacttctgagaatgttgttttatgctttattcttgtttgtttctcatcattggaagtgataaatctctttgatatcttcttaaatagtgcttcttgtttgcatcatctgaatgaaccatttttcatgcagatactaactgggcaccaagataatgctgaatttgctcttgccatgtgcccaacggaaccctttgtgctctccggaggtttgtgtttctgtaatttgtagagtccaatcctgtggtttgccagtttctcatacaaaagttcttctcttaggcaaggacaagtcagttgttttgtggagtatccaagatcacatcacaacgattgggacagattccaaatcatctggatctatcatcaaacagactggtgaaggtactgataagaatgagagtcctactgttggcccacgaggtgtatatcatggccatgaagatacagttgaagatgtggcattcagcccgacgaggtaacttcttagaacagactccttctattgatatcgtgtttgtttatgcatactgcagatattttcatgattttctaataatacttctggtgaacttttataccgtgaagtgcacaagaattctgcagtgttggtgatgattcttgccttatactatgggatgcgagaactggcacaaaccctgtcacgaaggtactctatcttttgaatcctatcaaaagtttgaagatttacctccttttgatattatatcttacttttttgttttccaggttgaaaaagcgcatgatgctgatcttcattgtgttgattggaatcctcatgacgacaatctgatcctgacagggtatggagaaatacatacaaatagatgattaatacatacttagtatctaattaagaaattgatgaatatttcaggtcagcagacaacactgtccggttgtttgatcgtaggaagcttaccgctaatggagttggttcgcctatttacaaatttgagggacacaaagctgctgttctttgtgttcaggtataatcaactttttttttttttttccttctttgtatgaagtatatctcttaacccactgacactatcttgttattcaattcagtggtctcctgataagtcatccgtctttgggagctctgcagaagatggtctcttgaacatctgggattatgacagggtgtgtacatagttcactcagatgtctaaaattaatctttcttcactatcatcactgaaacatattcattgtactcatgtttggtttgtttaattaaccatcaggtcagtaagaagtctgatcgtgcagctaaaagccccgctgggctcttcttccagcatgctggtcacaggttcttaaagacttatcttgatttttcttgattgctttctcattttacttgcttctaagttcccttgtttataaaccatattagggacaaagttgttgatttccactggaatgcttcagacccttggactattgtcagtgtttctgatgactgtgagactactggtggaggtggaacattgcaggtaaccttgaaatctttcttggtaccttgataagcaattttattgacataccgttaaatgttgtttatactttcttctatggcagatatggcggatgagtgacttgatttacagaccagaagaagaagtcgtggcagaattggagaagttcaagtcgcatgttatgacttgtgcctccaagccttaagagtaaagaaaacccattgtctatctatctatcgcctatggtaaactaatgcgggttttagcgaggagtcttggtttttgtaaggctggtttgtcttttgagatattggtggtagcttttaggacctttccatatcagttaggggtacatggttctggttcatgatcctgtttcatcagactcttaggtgctgttttgtt

SEQ ID NO: 7 - DNA, 1545 bp, GOSSYPIUM HIRSUTUM
atggagtctcctcaaccccaacaacagcaacaacaaggtcctaagaagagaggtcgaaaaccaaagccaaaggaagagaaagaacagcagcaacaacaaagtgctagtaaaatgaaagagggaaagaaagctcaccaacctagcgttgatgagaagtacactcagtggaagtctttggttcctgttctttatgactggcttgctaatcacaatctcgtttggccttctctttcttgccgctggggaccacagcttgagcaagctacttacaagaatcggcagcgtctctacctctctgaacagactgatggtagtgttccgaatactcttgtgattgctaattgtgaagttgtcaaacctagggttgctgctgctgagcacatatctcagtttaatgaaaaaacacgctcaccatttgtgaagaagtacaaaaccatcatacatcctggagaagtcaacagaatcaaggaacttccacagagctctaggattgtggcaactcacactgatagtcctgatgttcttatttgggatgtcgaggctcaacccaaccgccatgcggttcttggagcaacccattctcgcccagatttgattttgactgggcatcaagataatgctgaatttgctcttgcaatgtgtccaactgagccttatgtgctctctggagggaaggacaaatcagtggttttgtggagcatccaggaccacataacaacaatggctacagaccctactaaatctcctggatctggcggatcaatcatcaaacaaaacaagcctggggaaggtaatgacaaagctgctgatgggccttctttaggtcctcgtggtgtcttctgtgggcatgaggatacagttgaagatgttacattctgtccatcaagtgcacaagagttttgtagtgtaggtgatgattcctgcctcatactatgggatgcacgagttggcactggccccactgtcaaggttgaaaaggcacataatgctgatctccattgtgttgattggaacccccatgatgacaatcttatcctgactgggtctgcagatcatactgtacgtatgtttgatcgaagaaatcttacttctaatggaattggatcacccatttacaagtttgagggtcataaagctgctgttctatgcgtgcagtggtctccagacaaatcatctgtatttggcagttctgctgaggatgggctcttgaacatttgggactatgacaaggttggcaagaaggtggaacgtgcttcaagatctcctagtgcttctgcgggactgtttttccagcacgctggacacagggacaaagttgttgacttccattggaatgcatttgatccatggactgttgttagtgtgtttgatgactgtgaaacaactggtggaggagggacattgcaaatatggggcatgagtgatttgatttataggccggaaaaggaggttttagcttagctggagaaattcaagtcccatgtcattttgtgggcgacaaaggcattaaaaaggtttagtagccctaatggtatgcctatttga

SEQ ID NO: 8 - protein, 502 aa, GOSSYPIUM HIRSUTUM
  misc_feature (487)..(487): Xaa can be any naturally occurring amino acid
MESPQPQQQQQQGPKKRGRKPKPKEEKEQQQQQSASKMKEGKKAHQPSVDEKYTQWKSLVPVLYDWLANHNLVWPSLSCRWGPQLEQATYKNRQRLYLSEQTDGSVPNTLVIANCEVVKPRVAAAEHISQFNEKTRSPFVKKYKTIIHPGEVNRIKELPQSSRIVATHTDSPDVLIWDVEAQPNRHAVLGATHSRPDLILTGHQDNAEFALAMCPTEPYVLSGGKDKSVVLWSIQDHITTMATDPTKSPGSGGSIIKQNKPGEGNDKAADGPSLGPRGVFCGHEDTVEDVTFCPSSAQEFCSVGDDSCLILWDARVGTGPTVKVEKAHNADLHCVDWNPHDDNLILTGSADHTVRMFDRRNLTSNGIGSPIYKFEGHKAAVLCVQWSPDKSSVFGSSAEDGLLNIWDYDKVGKKVERASRSPSASAGLFFQHAGHRDKVVDFHWNAFDPWTVVSVFDDCETTGGGGTLQIWGMSDLIYRPEKEVLAXLEKFKSHVILWATKA

SEQ ID NO: 9 - DNA, 1542 bp, GLYCINE MAX
atggagactcctcctccccaacaaggcgtcgtgaagaagaaggagacaaggggccgaaaacccaaaccaaaggacgaacacgggaagggtttgaaggaaggtaggaaaacacaacaacaacaacaacaacaacaacaacatcatcatcagcagcagcaacagcaacaagatcaaccttcggtggacgagaaatacacgcagtggaagtcccttgttcctgtcctctacgactggctcgccaaccacaacctcgtctggccctctctctcttgcaggtggggcccccagcttgaacaagccacttacaagaatcgccagagactctacctttctgagcagactgatggtagtgtgccgaatactctggtgattgcgaattgcgaggttgtgaagcctagggttgctgctgctgagcacatttcgcagtttaatgaagaggcgcggtccccatttgtgaagaagtacaagaccatcatacatcctggtgaggtaaacagaattagggaattgccacaaaattccaagatagtggctacacatacagacagccctgatgtccttgtttgggatgttgaaagtcaacctaatcgccatgctgtccttggagctacaaactctcgtcctgatttgatattgactggacaccaagataatgcggaatttgctcttgcaatgtgcccaactgaaccctatgttctttcaggaggaaaggacaaaacagtggtgttgtggagtattgaagaccatataacatctgctgccacagactccaaatctggtgggtcaattatcaaacaaaactctaaatctggagaaggcaatgataaaactgctgatggccctactgttggaccacgaggcatttattgtgggcatgaggatacagttgaagatgtgactttctgcccatctagtgcacaggagttctgtagtgttggagatgattcttgtctcatcttatgggatgcacgtgttggctctagccctgtggttaaggttgagaaagctcataatgctgatcttcactgtgtggactggaatccccatgatgataatctgattcttactgggtcagcagataattctgttcgcatgtttgatcgccgcaatctcaccactaatggagttgggtcacccatccataaatttgagggtcacaaagctgctgttctttgtgttcagtggtctccagacaaatcatctgtatttggaagttcagctgaagatggtctcttaaacatttgggactatgagaaggttggtaaaaagatagagcgatctggaaaatcaataagttctcctccagggttgttttttcaacatgcaggtcatagggataaagttgttgacttccattggaatgcatatgatccatggacgattgttagtgtgtctgatgactgtgaaagtactggaggagggggaacgttgcagatatggcgcatgagtgatttgatctacagaccagaagatgaggttttggccgagctggagaaattcaaatctcatgttgtggcgtgtgcttcaaagactgaaaaatga

SEQ ID NO: 10 - protein, 498 aa, GLYCINE MAX
KETRGRKPKPKDEHGKGLKEGRKTQQQQQQQQQHHHQQQQQQQDQPSVDEKYTQWKSLVPVLYDWLANHNLVWPSLSCRWGPQLEQATYKNRQRLYLSEQTDGSVPNTLVIANCEVVKPRVAAAEHISQFNEEARSPFVKKYKTIIHPGEVNRIRELPQNSKIVATHTDSPDVLVWDVESQPNRHAVLGATNSRPDLILTGHQDNAEFALAMCPTEPYVLSGGKDKTVVLWSIEDHITSAATDSKSGGSIIKQNSKSGEGNDKTADGPTVGPRGIYCGHEDTVEDVTFCPSSAQEFCSVGDDSCLILWDARVGSSPVVKVEKAHNADLHCVDWNPHDDNLILTGSADNSVRMFDRRNLTTNGVGSPIHKFEGHKAAVLCVQWSPDKSSVFGSSAEDGLLNIWDYEKVGKKIERSGKSISSPPGLFFQHAGHRDKVVDFHWNAYDPWTIVSVSDDCESTGGGGTLQIWRMSDLIYRPEDEVLAELEKFKSHVVACASKT

SEQ ID NO: 11 - DNA, 1365 bp, PETUNIA HYBRIDA
atgaaggagagaaccggaaaagggggtcagccgtcggttgacgagaagtacacacagtggaagtctctcgttcccgttctctatgactggcttgctaaccataacctcgtatggccatcactttcctgcagatgggggcctctggttgagcaagcaacttacaagaatcgccaacgactttacctctcagagcagactgatggaagtgtgccaaacactttggttatagcaaattgtgaagttgttaaacctagggttgcagctgcagagcacatatcacagttcaatgaagaatcacgatctccttttgtcaagaagtacaaaaccattatacacccaggggaggttaacagaatcagagagcttcctcaaaataaaaacatagtggcaacccatactgatagtcctgaagttctaatttgggatgtggaagcccaacctaatcgacatgctgttcttggagctgttgcttcacgcccagatttgacattgattggacatagtgagaatgcagaatttgcactggcaatgtgccccactgaaccctttgtgctctctggaggaaaggacaaatctgtggtactgtggagtattcaagatcatatatcaacattgagcacagatgcacaaaaacctgctggtttcatcaagccagccactacatctattaaggctggtgataatccctctattcaagctcgtggaatcttccaaggccatgaggataccgttgaagatgttcagttctgcccatcaagttcacaggaattctgtagtgttggtgatgattcatgtctcattctatgggatgctcgagttggtactagtccagttgtaaaggttgaaaaagctcataatgctgatctccactgtgttgactggaatcctcatgatggcaactttattatcactggatctgcagataattctgtccgcttgtttgatcgacgtagtttaacttcgaatggagttggctcaccagtccatatctttgaaaatcacaaagcagcagttctttgtgtgcagtggtgtccagacaggtcctctgtatttgggagtactgcagaggatggtcgtttgaatatttgggattacgataaggttggtgagaaggacaatgagacacctgctccaggtttatttttccagcatgctgggcacagggataaaattgttgacttccactggaatgtggctgatccatggacaattgtaagtgtatctgatgactgtgactccacaggtggaggtggtacactacagatatggcggatgttggatttgctgtatcgtcctgaagaggaggctctggccgagcttcaaaagttcaaatcccatgtaagcaagtgcgctcgcaaaatctga

SEQ ID NO: 12 - protein, 454 aa, PETUNIA HYBRIDA
MKERTGKGGQPSVDEKYTQWKSLVPVLYDWLANHNLVWPSLSCRWGPLVEQATYKNRQRLYLSEQTDGSVPNTLVIANCEVVKPRVAAAEHISQFNEESRSPFVKKYKTIIHPGEVNRIRELPQNKNIVATHTDSPEVLIWDVEAQPNRHAVLGAVASRPDLTLIGHSENAEFALAMCPTEPFVLSGGKDKSVVLWSIQDHISTLSTDAQKPAGFIKPATTSIKAGDNPSIQARGIFQGHEDTVEDVQFCPSSSQEFCSVGDDSCLILWDARVGTSPVVKVEKAHNADLHCVDWNPHDGNFIITGSADNSVRLFDRRSLTSNGVGSPVHIFENHKAAVLCVQWCPDRSSVFGSTAEDGRLNIWDYDKVGEKDNETPAPGLFFQHAGHRDKIVDFHWNVADPWTIVSVSDDCDSTGGGGTLQIWRMLDLLYRPEEEALAELQKFKSHVSKCARKI

SEQ ID NO: 13 - DNA, 1548 bp, PISUM SATIVUM
atggagactcctccgtcgcagcaaggcgtagtaaagaagaaggaaactcgaggtcgaaaacctaaaccaaaagacgatcaacaacaaacacctgccaaaacactgaaagaagcgaaaaaggcgcaactacaacagcagcaacaacagcaactgcagcaacagcagcaacaacaacaacaccaagcttcggtagacgagaaatacactcaatggaagtctctcgttcccgttctctacgattggcttgcgaatcataaccttgtttggccttctctctcttgcaggtggggtcctcagcttgaacaagctacttataagaatcggcagcggctttatctttctgagcagactgatggtagtgtcccaaatactttggtgattgcgaattgtgaggttgtgaaaactagggttgcagctgcagagcatatttcacagtttaatgaggaggctcgctctccatttgttaagaagtacaagaccatcatacatcccggggaggtgaacagaattagggaattgccgcaaaattctaagatagtggctactcacacagacagccctgatgttctcatttgggatgttgaaagtcaacctaaccgtcacgctgtccttggagccacaaactctcgtccagatttgatattgaccggacaccaagacaatgctgagtttgctcttgcgatgtgcccaactgagccttatgtcctttcaggaggaaaagataaaacagtggtgttgtggagtattgaagaccatgtaacatctgctgctacggacaagtctggtggatccattatcaaaccgaactctaaatctggggaaggcaatgacaaaactgttgatagcccttctgtcgggccaagaggtatctactctgggcacgatgatactgttgaagatgtggccttttgcccttctagtgcgcaggaattctgtagtgttggagatgattcttgtctcatattatgggatgcacgtgttggctctagtcctgttgttaaggttgaaaaagctcataatgccgatcttcactgtgttgactggaatccccatgatgataatctgattcttactgggtcggcagataattctgttcgcttgtttgatcggcgcaatctcacctctaatggggttgggtctcctattcataaatttgaggctcataaagctgccgttctttgtgttcagtggtctccagacaaatcatctgtatttggaagttcagcagaagacggtctcttaaacatttgggattatgagaaggttggtaaaaagattgagcgagctggaaaaacaataaactctcctccagggttgtttttccaacatgctggtcatagagacaaagttgttgacttccattggaatgcttatgatccatggacaattgtaagtgtgtctgatgattgtgaaagtactggtggaggaggaacattgcagatatggcgcatgagtgatttgctctatagaccagaggatgaggttttggcagagttggagaaattcaaatctcacgtggtggcttgtgctgcaaagaccgatacataa

SEQ ID NO: 14 - protein, 515 aa, PISUM SATIVUM
METPPSQQGVVKKKETRGRKPKPKDDQQQTPAKTLKEAKKAQLQQQQQQQLQQQQQQQQHQASVDEKYTQWKSLVPVLYDWLANHNLVWPSLSCRWGPQLEQATYKNRQRLYLSEQTDGSVPNTLVIANCEVVKTRVAAAEHISQFNEEARSPFVKKYKTIIHPGEVNRIRELPQNSKIVATHTDSPDVLIWDVESQPNRHAVLGATNSRPDLILTGHQDNAEFALAMCPTEPYVLSGGKDKTVVLWSIEDHVTSAATDKSGGSIIKPNSKSGEGNDKTVDSPSVGPRGIYSGHDDTVEDVAFCPSSAQEFCSVGDDSCLILWDARVGSSPVVKVEKAHNADLHCVDWNPHDDNLILTGSADNSVRLFDRRNLTSNGVGSPIHKFEAHKAAVLCVQWSPDKSSVFGSSAEDGLLNIWDYEKVGKKIERAGKTINSPPGLFFQHAGHRDKVVDFHWNAYDPWTIVSVSDDCESTGGGGTLQIWRMSDLLYRPEDEVLAELEKFKSHVVACAAKTDT

SEQ ID NO: 15 - DNA, 1503 bp, POPULUS
atggaacctgctgccactactgctcaagtgccaaagaagagaggccgaaaaccgaagcccaaagaagatcaacagcagcagcagcagcagcaaagtgctggtgcccgaatgaaagagggaaggaaggcacagcaaccctctattgatgacaagtacactcagtggaagtctttggttcctgttctttatgactggctagccaatcacaacctcgtctggccatctctctcttgccggtggggcccacagcttgagcaagctacttacaagaatcgccagcgcctctatctctccgagcagactgatggtagtgttccaaatactttggtcattgcaaattgtgatgttgtcaagtctagagttgctgctgcggaacacatatctcagtttaatgaagaagcacgctctccatttgttaagaagtacaagaccatcatacatcctggagaggtaaacagaatcagagaactcccccagaatagtaagatagtggctactcatactgacagccctgatgttcttatatgggatgttgaagcacagcctaaccgccatgctgttcttggagctacaaattctcgtccagatttgattttgactggacatcaagacaatgctgagtttgcccttgcaatgtgcccaactgatccctatgtgctttctggagggaaggacaagttcgtagttttgtggagtatccaggaccatataacatcatctgcctctgatccagcaactaagtctccaggatctggtggatcaatcattaaaaagactggggatggcagtgataaagccactgatggcccttctgttggaccgcgaggtatctaccaagggcatgaggatacagttgaagacgtggcattctgtccatccagtgcgcaggagttttgtagtgtaggagatgattcttgccttatattatgggatgctagagctggcactagcccagctataaaggttgaaagggcacataatgctgatcttcactgtgttgattggaatcctcaagatgataatctcattttaactgggtctgcagatacttctgtttgcatgtttgatcgaaggaatctcacttctaatggagttggtttgccagtctataaatttgagggtcacaaagctgctgttctctgtgtacagtggtctccagataaggcatctgtttttgggagttctgcggaggatggtctcttgaatatttgggattatgagaaggttggcaaaaagacagagcgtccgactagagctccaagttctcctgcaggattgtttttccagcatgctgggcacagggataaagttgttgatttccattggaatgcatctgatccttggacggtggttagtgtctctgatgactgtgataccactggcgggggagggacactgcagatatggcgcatgagtgatttgatctacaggccagaagatgaggttttggcagagcttgagaagttcaagtcacatgtggtttcctgtgcttcgaagccttga

SEQ ID NO: 16 - protein, 500 aa, POPULUS
MEPAATTAQVPKKRGRKPKPKEDQQQQQQQQSAGARMKEGRKAQQPSIDDKYTQWKSLVPVLYDWLANHNLVWPSLSCRWGPQLEQATYKNRQRLYLSEQTDGSVPNTLVIANCDVVKSRVAAAEHISQFNEEARSPFVKKYKTIIHPGEVNRIRELPQNSKIVATHTDSPDVLIWDVEAQPNRHAVLGATNSRPDLILTGHQDNAEFALAMCPTDPYVLSGGKDKFVVLWSIQDHITSSASDPATKSPGSGGSIIKKTGDGSDKATDGPSVGPRGIYQGHEDTVEDVAFCPSSAQEFCSVGDDSCLILWDARAGTSPAIKVERAHNADLHCVDWNPQDDNLILTGSADTSVCMFDRRNLTSNGVGLPVYKFEGHKAAVLCVQWSPDKASVFGSSAEDGLLNIWDYEKVGKKTERPTRAPSSPAGLFFQHAGHRDKVVDFHWNASDPWTVVSVSDDCDTTGGGGTLQIWRMSDLIYRPEDEVLAELEKFKSHVVSCASKP

SEQ ID NO: 17 - DNA, 1389 bp, VITIS VINIFERA
atgaaggagacgagcacagccggcgagacgtcgatttcggtggacgagaggtatacccagtggaagtctctcgttccgactgatggcagtgttccgaataccttagttatagcaaactgtgaagttgttaaacctagggtagctgccgcagagcatattgctcagttcaacgaggaagcgcggtcaccctttgtaaagaagttcaaaacaattatacatccaggagaggtgaaccgaatcagggaactgccacagaatagtaagatagtggccacacacactgacagtcctgatgtcctcatttgggatgttgaaactcaacctaatcgtcatgctgtattgggaactccagaatctcgtccagatttgttttggtttacattttcactagctgtgaatttacatgatgcatggagcccacccaacaaatcgggagaaagatttagtggcacctttgaaccccgtataacaggttctgcttatttagagactacagaggggaaggttttgttcctgatattaactgggcataaagataatgcagaatttgctcttgccatgtgtccaactgaaccattggtgctctctggaggcaaggataagtctgtggtgttgtggagtattcaggatcacatctcgactttggcagcagatccagggtcagcaaagagtacctctaaggctggtggtggtaatgataaacctgtagaaagcccttctataggagcacgtggtatctaccaagggcatgatgatactgttgaagatgtgcaattctgcccattaagcgcacaggagttctgtagcgtaggtgatgattcttgccttatcttatgggatgcaagatctggcaccactccagccatcaaggtcgagaaagctcataatgctgatcttcactgtgttgattggaatcctcatgacataaatcttattctaactggatcggctgacaatactgttcgcatgtttgatcgccgaaagcttacatctggtggaattggatcaccaatccatacatttgagggccatactgctgctgtcctttgtgtacagtggtctccggacaaggcttcgatctttgggagttctgcagaagatggtatcttaaacctctggaatcatgaaaagattgataagaagcaagctcccaacgctcctccaggtttattcttcagacatgctggccatagggataaggtcgtggacttccattggaatgcatcggatccatggacaatcgttagcgtatctgatgatggtgaaagtactggtggaggcggtacactccagatatggcggatgatcgatctgatttacaggaacgaagatgaggtgctggctgagctcgataacttcaaagctcatttggccacatgctccccctga

SEQ ID NO: 18 - protein, 462 aa, VITIS VINIFERA
MKETSTAGETSISVDERYTQWKSLVPTDGSVPNTLVIANCEVVKPRVAAAEHIAQFNEEARSPFVKKFKTIIHPGEVNRIRELPQNSKIVATHTDSPDVLIWDVETQPNRHAVLGTPESRPDLFWFTFSLAVNLHDAWSPPNKSGERFSGTFEPRITGSAYLETTEGKVLFLILTGHKDNAEFALAMCPTEPLVLSGGKDKSVVLWSIQDHISTLAADPGSAKSTSKAGGGNDKPVESPSIGARGIYQGHDDTVEDVQFCPLSAQEFCSVGDDSCLILWDARSGTTPAIKVEKAHNADLHCVDWNPHDINLILTGSADNTVRMFDRRKLTSGGIGSPIHTFEGHTAAVLCVQWSPDKASIFGSSAEDGILNLWNHEKIDKKQAPNAPPGLFFRHAGHRDKVVDFHWNASDPWTIVSVSDDGESTGGGGTLQIWRMIDLIYRNEDEVLAELDNFKAHLATCSP

SEQ ID NO: 19 - DNA, 1272 bp, SORGHUM BICOLOR
  misc_feature (723)..(723): n is a, c, g, or t
atgaaggagagaagcggctccagggcggcggtggacgagcgctacgcgcagtggaagtcgctcatccccgtcctctacgactggtttgctaaccacaacctcgtctggccatccctctcctgccggtgggggccacagtttgagaaagctacctacaagaatcgtcaacgcctttacctatctgagcagacggatgggagtgtgcctaatactctagttatcgcaaactgtgaagttgtgaagccacgggttgcagctgctgaacatatctcgcagtttaacgaggaagcacgatcaccttttgttaagaagtacaagaccatagttcatcctggtgaagttaacagaatcagggagcttccacagaacagtaagatcatagccactcacactgacagtccagatgtacttatttgggatgttgaagcacagccaaatagacatgccgtcctaggagcttctgaatctcgccctgatctgatattaacgggacataaggaaaatgcggaatttgcgcttgctatgtgcccagcagaaccatatgtcctatcaggaggaaaggacaaatctgttgtcttgtggagcatccaagaccacatatctgcccttggggattcctcgtcttctcctggagcatctggcagcaagcagtctggtaaatctgcaactgaaaaggagagccctaaagttgatcctagaggtatattctgtgggatgctcganactggcactggcccagctgttaaggttgagaaagctcacagtggagatgttcattgtgttgattggaatccccttgacgttaactatatcttaactggttctgccgataactctgtccgaatgtgggatcgtcgtaagctgggttcgggaggagctagttctccaattcataaatttgagggccataaagctgctgttctttgtgttcagtggtcacctgacagagcatctgtttttggaagttctgcggaagatggtttcttaaacgtgtgggatcatgagaaggttgggactaagaaaaatactaatgtcccagctgggcttttcttccagcatgctggtcatagggataagattgtagacttccactggaattcgtcagatccttggacaattgtcagtgtatctgatgatggtgagagcactggtggaggcggaacactgcagatatggcgcatgagtgatttgatctaccgccctgaggatgaagttcttacagaactggagaatttcaaggctcacttggccagctgtgctccaaggacttga

SEQ ID NO: 20 - protein, 423 aa, SORGHUM BICOLOR
MKERSGSRAAVDERYAQWKSLIPVLYDWFANHNLVWPSLSCRWGPQFEKATYKNRQRLYLSEQTDGSVPNTLVIANCEVVKPRVAAAEHISQFNEEARSPFVKKYKTIVHPGEVNRIRELPQNSKIIATHTDSPDVLIWDVEAQPNRHAVLGASESRPDLILTGHKENAEFALAMCPAEPYVLSGGKDKSVVLWSIQDHISALGDSSSSPGASGSKQSGKSATEKESPKVDPRGIFCGMLELALAQLLRLRKLTVEMFIVVDWNPLDVNYILTGSADNSVRMWDRRKLGSGGASSPIHKFEGHKAAVLCVQWSPDRASVFGSSAEDGFLNVWDHEKVGTKKNTNVPAGLFFQHAGHRDKIVDFHWNSSDPWTIVSVSDDGESTGGGGTLQIWRMSDLIYRPEDEVLTELENFKAHLASCAPRT

SEQ ID NO: 21 - DNA, 1362 bp, ZEA MAYS
atgaaagagagaggcggctccagcgcggcggtggacgagcgctacgcgcagtggaagtcgctcatccccgtcctctacgactggtttgccaaccacaacctcgtctggccatccctctcctgccggtgggggccacagtttgagaaagctacctacaagaatcgtcagcgcctgtacctatctgaacagacggatgggagtgtgcctaatactctggttatcgcaaattgtgaagttgtgaaacctagggttgcagctgctgaacatatctcacagttcaatgaggaagcacggtcaccttttgtaaagaagtacaaaactatagttcatcctggtgaggttaacagaatcagggaacttccacagaacagtaagatcatagccactcacactgacagtccagatgtacttatttgggatgttgaagcacaaccaaatagacatgctgtcctaggagcaagcgagtctcgccctgatctgatattaacaggacataaggaaaatgcggaatttgcgcttgccatgtgtccagcagaaccatatgtcctatcaggaggaaaggacaaatctgttgtcttgtggagcatccaagaccatatatctgcccttggggattcctcgtcttctcctggagcatctggcagcaagcagtctattaaaactgcaaatgaaaaggagagccctaaagttgatcctagaggtatatttcatggacatgacagcacggttgaagatgttcagttctgcccttccagtgcgcaggagttttgtagtgtgggtgatgatgcttgtcttattctgtgggatgctcgaactggcactgccccagctgttaaggttgagaaagctcacagtggagatgttcattgtgttgattggaatccccttgatgttaactatatcttaactgggtctgccgataactctgtccgaatgtgggatcgtcggaatctgggttcaggaggagctggttctccaattcacaaatttgaaggccataaagctgctgttctttgtgttcagtggtcacctgacagagcatctgtttttggaagttctgcagaagatggtttcttaaacgtgtgggatcatgagaaggtggggaagaagaaaaattctaatgtcccagccgggcttttctttcagcacgctggtcatagggataagattgtagacttccactggaattcgtcagatccttggacaattgtcagtgtctctgatgatggcgagagcactggtggaggtggaacactgcagatttggcgcatgagtgatttgatctaccgcccagaggatgaagttcttgcagagctggagaatttcaaggctcacttggccagctgtgcaccgaagaattga

SEQ ID NO: 22 - protein, 453 aa, ZEA MAYS
MKERGGSSAAVDERYAQWKSLIPVLYDWFANHNLVWPSLSCRWGPQFEKATYKNRQRLYLSEQTDGSVPNTLVIANCEVVKPRVAAAEHISQFNEEARSPFVKKYKTIVHPGEVNRIRELPQNSKIIATHTDSPDVLIWDVEAQPNRHAVLGASESRPDLILTGHKENAEFALAMCPAEPYVLSGGKDKSVVLWSIQDHISALGDSSSSPGASGSKQSIKTANEKESPKVDPRGIFHGHDSTVEDVQFCPSSAQEFCSVGDDACLILWDARTGTAPAVKVEKAHSGDVHCVDWNPLDVNYILTGSADNSVRMWDRRNLGSGGAGSPIHKFEGHKAAVLCVQWSPDRASVFGSSAEDGFLNVWDHEKVGKKKNSNVPAGLFFQHAGHRDKIVDFHWNSSDPWTIVSVSDDGESTGGGGTLQIWRMSDLIYRPEDEVLAELENFKAHLASCAPKN

SEQ ID NO: 23 - DNA, 1362 bp, ZEA MAYS
atgaaggagagaggcggctccagggcggcagtggacgagcgctacgcgcagtggaagtcgctcatccccgttctctacgactggtttgccaaccacaacctcgtttggccatccctctcctgccggtgggggccacagtttgagaaagctacctacaagaatcgtcaacgcctttacctatctgaacagaccgatgggagtgtgcctaatactctggttatcgcaaactgtgaagttgtgaaaccaagggttgcagctgctgaacatatctcgcagtttaacgaggaagcacgatcaccttttgtaaagaagtacaagactatagttcatcctggtgaagttaacagaatcagggagcttccacagaacagtaagatcatagccacacacactgacagtccagatgtacttgtttgggatgttgaagcacaaccaaatagacatgctgtcttaggagcttctgaatctcgtcctgatctgatattaacgggacaccaggaaaatgcagaatttgcgcttgccatgtgtccagcagaaccatatgtcctgtcaggaggaaaggacaaatttgttgtcttgtggagcatccaagaccacatatctgcccttggggattcctcgtcttctcccggagcatctggcagcaagcagtctggcaaaattgcaaatgaaaaggagagtcctaaagttgatcctagaggtatattccatggccatgacagtactgttgaagatgttcagttctgcccttccagtgcgcaggagttttgtagtgtgggtgatgatgcttgtcttattctgtgggatgctcgaactggcactgacccagctgttaaggttgagaaagctcacagtggagatgttcattgtgttgattggaatccccttgatgttaactatatcttaactggttctgccgataactctgtccgaatgtgggatcgtcgcaatctgggttcgggaggagctggttctccaattcacaaatttgagggccataaagctgctgttctttgtgttcagtggtcacctgacagagcatctgtttttggaagttctgcagaagatggtttcttaaatgtttgggaccatgagaaggttgggaagaagaaaaattctaatgtcccagctgggcttttctttcagcatgctggtcatagggataagatcgtagacttccactggaattcgtcagatccttggacaattgtcagtgtctcagatgatggtgagagcactggtggaggtggaacactgcagatatggcgaatgagtgatttgatctaccgcccagaggatgaagttcttacagagctggagaatttcaaggctcacctggccagttgcgctccgaggaattga

SEQ ID NO: 24 - protein, 453 aa, ZEA MAYS
MKERGGSRAAVDERYAQWKSLIPVLYDWFANHNLVWPSLSCRWGPQFEKATYKNRQRLYLSEQTDGSVPNTLVIANCEVVKPRVAAAEHISQFNEEARSPFVKKYKTIVHPGEVNRIRELPQNSKIIATHTDSPDVLVWDVEAQPNRHAVLGASESRPDLILTGHQENAEFALAMCPAEPYVLSGGKDKFVVLWSIQDHISALGDSSSSPGASGSKQSGKIANEKESPKVDPRGIFHGHDSTVEDVQFCPSSAQEFCSVGDDACLILWDARTGTDPAVKVEKAHSGDVHCVDWNPLDVNYILTGSADNSVRMWDRRNLGSGGAGSPIHKFEGHKAAVLCVQWSPDRASVFGSSAEDGFLNVWDHEKVGKKKNSNVPAGLFFQHAGHRDKIVDFHWNSSDPWTIVSVSDDGESTGGGGTLQIWRMSDLIYRPEDEVLTELENFKAHLASCAPRN

SEQ ID NO: 25 - DNA, 1362 bp, ORYZA SATIVA
atgaaggagaaggggtcgagggcggcggtggacgagcgctacgcgcagtggaagtcgctcatcccggtgctctacgactggttcgccaaccacaacctcgtctggccgtccctctcctgccggtggggaccccagtttgagaaggctacctacaagaatcgtcagcgtctttacctttcagagcagacggatgggactgtccctaatactctggttattgcaaattgtgaagttgtcaaaccaagggttgcagctgctgaacatatatctcagttcaatgaggaagcacgatcaccttttgtaaagaagtacaagactataattcatccaggcgaggtgaacaggattagggagcttccgcagaacagtaagatcattgccactcataccgacagcccagatgttctcatttgggatgtcgaggcccagccaaatagacaagctcaactggcacagatggagtctcgtcctgatttgatattaagaggacataaggatattgctgagtttgcgcttgctatgtgcccagctgagccatatgtgttatctggaggaaaagacaaatctgttgtatggtggagcatccaagaccacatatctgcactgggagattcctcgaaaactgaatcttctccaggggcatcaggaagcaagggaaaaactgcaaatgataaggatagtcctaaagttgatcctcgtggtatctttcttggccacgacagtactgttgaagatgtccagttctgcccttctagtgcacaggagttttgtagtgtaggcgatgattcttgtcttattctttgggatgcccggagtggtacaggcccagctgttaaggttgagaaagctcatggtggtgatgttcattgtgttgactggaacctccatgatgttaactatatcttaactggttctgcggataattctgtccgtatgtgggaccggagaaatctgggttctggaggagctggcattccagttcacaaatttgagggtcataaagctgctgttctttgtgttcagtggtcacctgacaaggcatctgtatttggaagctctgcggaagacggcttcttaaatgtgtgggatcatgagaaggttggaaataagaaaaatcccaacgcacctgctgggcttttctttcaacatgctggtcatagggataagattgtagacttccactggaattcttcggatccttggactattgtgagtgtgtctgatgatggtgagagtactggtggaggtggaacattgcagatatggcgcatgagtgacctgatataccgtccagaagatgaagttcttgcagagctggagaacttcaagactcacttggccagctgtgctccaaggagctga

SEQ ID NO: 26 - protein, 453 aa, ORYZA SATIVA
MKEKGSRAAVDERYAQWKSLIPVLYDWFANHNLVWPSLSCRWGPQFEKATYKNRQRLYLSEQTDGTVPNTLVIANCEVVKPRVAAAEHISQFNEEARSPFVKKYKTIIHPGEVNRIRELPQNSKIIATHTDSPDVLIWDVEAQPNRQAQLAQMESRPDLILRGHKDIAEFALAMCPAEPYVLSGGKDKSVVWWSIQDHISALGDSSKTESSPGASGSKGKTANDKDSPKVDPRGIFLGHDSTVEDVQFCPSSAQEFCSVGDDSCLILWDARSGTGPAVKVEKAHGGDVHCVDWNLHDVNYILTGSADNSVRMWDRRNLGSGGAGIPVHKFEGHKAAVLCVQWSPDKASVFGSSAEDGFLNVWDHEKVGNKKNPNAPAGLFFQHAGHRDKIVDFHWNSSDPWTIVSVSDDGESTGGGGTLQIWRMSDLIYRPEDEVLAELENFKTHLASCAPRS

SEQ ID NO: 27 - DNA, 1506 bp, ORYZA SATIVA
atgaaggagaaggggtcgagggcggcggtggacgagcgctacgcgcagtggaagtcgctcatcccggtgctctacgactggttcgccaaccacaacctcgtctggccgtccctctcctgccggtggggaccccagtttgagaaggctacctacaagaatcgtcagcgtctttacctttcagagcaggcaagtgctcccaaaataagttgttctttttctttcttttgctgtagggatgttttgaagctcaaatttatgacggatgggactgtccctaatactctggttattgcaaattgtgaagttgtcaaaccaagggttgcagctgctgaacatatatctcagttcaatgaggaagcacgatcaccttttgtaaagaagtacaagactataattcatccaggcgaggtgaacaggattagggagcttccgcagaacagtaagatcattgccactcataccgacagcccagatgttctcatttgggatgtcgaggcccagccaaatagacaagctcaactggcacagatggagtctcgtcctgatttggtaccacctgactctcgccctgatttgatattaagaggacataaggatattgctgagtttgcgcttgctatgtgcccagctgagccatatgtgttatctggaggaaaagacaaatctgttgtatggtggagcatccaagaccacatatctgcactgggagattcctcgaaaactgaatcttctccaggggcatcaggaagcaagggaaaaactgcaaatgataaggatagtcctaaagttgatcctcgtggtatctttcttggccacgacagtactgttgaagatgtccagttctgcccttctagtgcacaggagttttgtagtgtgggcgatgattcttgtcttattctttgggatgcccggagtggtacaggcccagctgttaaggttgagaaagctcatggtggtgatgttcattgtgttgactggaacctccatgatgttaactatatcttaactggtgttcatttggtacaagaagccgtggatacacttcttgatattctgcggataattctgtccgtatgtgggaccggagaaatctgggttctggaggagctggcattccagttcacaaatttgagggtcataaagctgctgttctttgtgttcaggcattggtcacctgacaaggcatctgtatttggaagctctgcggaagacggtttcttaaatgtgtgggatcatgagaaggttggaaataagaaaaatcccaacgcacctgctgggcttttctttcaacatgctggtcatagggataagattgtagacttccactggaattcttcggatccttggactattgtgagtgtgtctgatgatggtgagagtactggtggaggtggaacattgcagatatggcgcatgagtgacctgatataccgtccagaagatgaagttcttgcagagctggagaacttcaagactcacttggccagctgtgctccaaggagctga

SEQ ID NO: 28 - protein, 501 aa, ORYZA SATIVA
MKEKGSRAAVDERYAQWKSLIPVLYDWFANHNLVWPSLSCRWGPQFEKATYKNRQRLYLSEQASAPKISCSFSFFCCRDVLKLKFMTDGTVPNTLVIANCEVVKPRVAAAEHISQFNEEARSPFVKKYKTIIHPGEVNRIRELPQNSKIIATHTDSPDVLIWDVEAQPNRQAQLAQMESRPDLVPPDSRPDLILRGHKDIAEFALAMCPAEPYVLSGGKDKSVVWWSIQDHISALGDSSKTESSPGASGSKGKTANDKDSPKVDPRGIFLGHDSTVEDVQFCPSSAQEFCSVGDDSCLILWDARSGTGPAVKVEKAHGGDVHCVDWNLHDVNYILTGVHLVQEAVDTLLDILRIILSVCGTGEIWVLEELAFQFTNLRVIKLLFFVFRHWSPDKASVFGSSAEDGFLNVWDHEKVGNKKNPNAPAGLFFQHAGHRDKIVDFHWNSSDPWTIVSVSDDGESTGGGGTLQIWRMSDLIYRPEDEVLAELENFKTHLASCAPRS

SEQ ID NO: 29 - DNA, 1503 bp, MEDICAGO TRUNCATULA
atggagacaccaccctcaaccggcgccgtgaagaagaaggagacaagaggacgcaaacctaaacccaaggaagacaagagagaagaaccatcacaagtgaagtcaccgagagaatcgaagaaggaaaagcaacagcaacagcttcatcagcagcagcaacaacaagcttctgttgatgagaagtactctcaatggaaatctcttgttcctgttctttatgattggcttgctaatcataacctcgtttggccttctctctcttgccggtggggtcctcagcttgaacaggctacttacaagaatcgtcagaggctttatctttcggagcagactgatggaagtgttccgaatactttggtgattgcgaattgtgaggttgtgaagactagggttgcagctgctgaacacatttctcagtttaatgaagaggctcgctctccatttgtcaagaagtacaagactatcatacatccaggcgaggtgaacagaattagggaattgccacaaaattctaagattgtggccactcacacagacagccctgatgttctcatttgggatgttgaaagtcaacctaaccgtcatgctgtccttggagctacaaactctcgtccagatctgatattgactggacaccaagataatgcagagtttgctcttgcaatgtgccctactcagccctatgtgctttctggaggaaaagacaaaacagtcgtgttgtggagtattgaagaccatataacatctgctgctacagactccaacaagtctggtggatccattgccaaaactgctgatagccctactgtcggaccaagaggtatctactctgggcatgaggatactgttgaagacgtggctttttgcccttctagtgcgcaggagttctgtagtgttggagatgattcttgtctcatattatgggatgcacgtgttggctctagtcctgtggttaaggttgaaaaagctcatgatgctgatcttcactgtgttgactggaatccccatgatgataatctgattcttactgggtcggcagataattctattcgcatgtttgatcgtcgcaatctcacttctaatggggttgggtctcctatccacaaatttgaggctcataaggctgctgtcctttgtgttcagtggtctccagacaaatcatctgtatttggaagttcagcagaagatggtctcctgaacatttgggattatgagaaggttggcaagaagatagagcgagctggaaaaacaataaattctcctccagggttgtttttccaacatgctggtcatagagacaaagttgttgactttcactggaatgcacatgatccatggacacttgttagtgtgtctgatgattgcgaaagtactggtggagggggaacattgcagatatggcgcatgagtgatttgctctacagaccagaggatgaggttttggcagagctggagaaattcaaatctcatgtggtggcttgtgcagcaaagaccgacaaataa

SEQ ID NO: 30 - protein, 500 aa, MEDICAGO TRUNCATULA
METPPSTGAVKKKETRGRKPKPKEDKREEPSQVKSPRESKKEKQQQQLHQQQQQQASVDEKYSQWKSLVPVLYDWLANHNLVWPSLSCRWGPQLEQATYKNRQRLYLSEQTDGSVPNTLVIANCEVVKTRVAAAEHISQFNEEARSPFVKKYKTIIHPGEVNRIRELPQNSKIVATHTDSPDVLIWDVESQPNRHAVLGATNSRPDLILTGHQDNAEFALAMCPTQPYVLSGGKDKTVVLWSIEDHITSAATDSNKSGGSIAKTADSPTVGPRGIYSGHEDTVEDVAFCPSSAQEFCSVGDDSCLILWDARVGSSPVVKVEKAHDADLHCVDWNPHDDNLILTGSADNSIRMFDRRNLTSNGVGSPIHKFEAHKAAVLCVQWSPDKSSVFGSSAEDGLLNIWDYEKVGKKIERAGKTINSPPGLFFQHAGHRDKVVDFHWNAHDPWTLVSVSDDCESTGGGGTLQIWRMSDLLYRPEDEVLAELEKFKSHVVACAAKTDK

SEQ ID NO: 31 - DNA, 1440 bp, MEDICAGO TRUNCATULA
atgaatgaatcgaagaaagagggaacaatttcaacaaccccactctcaatcgaagaaagatacagtcaatggaagtcactcgttccagtcctctacgactggctagcgaatcacaatctagtatggccatcacaatcatgccgttggggttcacttcttgatcatgctacttacaagaatcgtcatcgtctttatctttctgaacaaactgatggaactgctcccaatacgcttgttattgctacttgtgaaattgttaaacctagggttgctgctgctgaacatattgctatgtttaatgaagaggcgcgttctccatttgttaagaaggttaaaactattcttcatcccggtgaggtaaatagaatcagagaactcccgtcaaatactaatatagttgccacacatacagatagtccaaatgttatgatttggaatgttgaatctcaacctaaccggaataatgctgctctggatgctcccacttccatcccagacttggtattaactggacataaggataatgctgaatttgcactagctatgtgttcaactgagccctttgttctttccggagggagagacaagcttgtggtgttatggagtattcatgatcatattgcaactttagctacagaagaagaacccgatgttaatgagggctctaatgttgggggaaatagcgagaaagctgcacaaagcccgtctgttggagcaaggggcgtctaccggggtcataaagacactgttgaagatgtgcagttttgcccctcaagtgcacaggagttctgtagtgtaggtgatgattcttgtctcatactctgggatgcgcgtgttggatcttttccagctgtcaaggttgagaaggcacatgatggagatgtacattgcgttgattggaatactcatgacatcaattttattctgactggctctgctgataacacagttcgcatgtttgatcgccggaaactaaacaatcgtggaggaattgggtctcctgtttataaatttgaaggccatgatgaaccagtcctctgtgtacagtggaatcctgctaaatcatctgtatttggaagtggtgccgaagatggaattataaacatctgggaccatgaaaaggttggtaaaacatcaggttctgctgatactacagtaccagagacttcccctggtttattctttcgtcatgcagggcatagggataaggttgttgactttcattggaatgcatctgatccatggacaattgttagtgtatctgatgattgtgcaagcactggtggaggtggcaccctgcagatctggcggatgatggatctaatttatcgaccagaggacgaggtgatggctgagctggataagttcaaatctcatatcttaggatgcgacactgtcactgacagtgccactgaccctgccactccatcacatacgtga

SEQ ID NO: 32 - protein, 479 aa, MEDICAGO TRUNCATULA
MNESKKEGTISTTPLSIEERYSQWKSLVPVLYDWLANHNLVWPSQSCRWGSLLDHATYKNRHRLYLSEQTDGTAPNTLVIATCEIVKPRVAAAEHIAMFNEEARSPFVKKVKTILHPGEVNRIRELPSNTNIVATHTDSPNVMIWNVESQPNRNNAALDAPTSIPDLVLTGHKDNAEFALAMCSTEPFVLSGGRDKLVVLWSIHDHIATLATEEEPDVNEGSNVGGNSEKAAQSPSVGARGVYRGHKDTVEDVQFCPSSAQEFCSVGDDSCLILWDARVGSFPAVKVEKAHDGDVHCVDWNTHDINFILTGSADNTVRMFDRRKLNNRGGIGSPVYKFEGHDEPVLCVQWNPAKSSVFGSGAEDGIINIWDHEKVGKTSGSADTTVPETSPGLFFRHAGHRDKVVDFHWNASDPWTIVSVSDDCASTGGGGTLQIWRMMDLIYRPEDEVMAELDKFKSHILGCDTVTDSATDPATPSHT

SEQ ID NO: 33 - DNA, 1419 bp, SILENE LATIFOLIA
atgacggagaaagggaaaggaggcaagaaagcattgagtgttgatgagaaatacagtcaatggaaatcacttgttcctattttatatgattggcttgccaatcataacctcgtttggccttctctctcttgcagatggggcccgttgattgaggaggcaacttacaagaaccgccaacgtctttatctctcagaacagactgatgggagtgttcctaataccctcgtgattgccaatgttgaagtcgtgaaacccagggttgctgctgcagaacatatatcgaagttcaatgaagaggctcgttctccctttgtgaggaagttcaaaacgataatacatcccggtgaggtaaacaggattagggagctcccccagaacagtaatatagttgcaactcatactgacagtccagatgtttatatttgggatctggaaagccaacctaatcgtcctgctaatttgggaacacctgcctctcgtccagatttgactctgactggtcatcaagataatgctgaattcgctcttgcaatgtgctcttctgaaccgctggtgctttctggaggaaaggataagtcagttgttttgtggagcatccatgatcatatctcaacattggcaacagaaccagggtcagcaaaatctcccaacagtggcagcaacataaagaaagctggtaatggaaattcagataatccttctattggacctcgaggcatctatctgggacatgaggatactgttgaagatgttcaattttgcccctcaagtgcacagcagttttgcagcgtgggtgatgactcctgtctcatactatgggatgcaagagctggattgacgccagtgactaaggttgagaaagctcacaatgctgatcttcactgtgttgactggaatccacatgatgaaaacctcattctaactgggtcagctgatagttctattaatttgtttgaccgccgtaatcttactgctagtggagttggatcacctgttcataaatttcagggtcatgatgctcctgtcctttgtgttcagtggtcaccgcataatagatcgatttttggcagtgctgctgaagatggcttgctaaacatatgggactatgaaaaggttagtaagatggaaacagagagcggaggtaaaaagtcaaaccatcctgcaggcttgtttttcagacatgctggacacagggacaaagtggtggacttccactggaattcgatagatccatggactctggttagtgtatcagatgactgttcaagctctgctggaggtggaacattgcagatatggcgcataatagacttgctgtacagaccagaagaggaagttttggctgagctggacaagttcagatcccatgtagccgcttgctcgcctactcctaccaaagatgttaatcattctgcctga

SEQ ID NO: 34 - protein, 472 aa, SILENE LATIFOLIA
MTEKGKGGKKALSVDEKYSQWKSLVPILYDWLANHNLVWPSLSCRWGPLIEEATYKNRQRLYLSEQTDGSVPNTLVIANVEVVKPRVAAAEHISKFNEEARSPFVRKFKTIIHPGEVNRIRELPQNSNIVATHTDSPDVYIWDLESQPNRPANLGTPASRPDLTLTGHQDNAEFALAMCSSEPLVLSGGKDKSVVLWSIHDHISTLATEPGSAKSPNSGSNIKKAGNGNSDNPSIGPRGIYLGHEDTVEDVQFCPSSAQQFCSVGDDSCLILWDARAGLTPVTKVEKAHNADLHCVDWNPHDENLILTGSADSSINLFDRRNLTASGVGSPVHKFQGHDAPVLCVQWSPHNRSIFGSAAEDGLLNIWDYEKVSKMETESGGKKSNHPAGLFFRHAGHRDKVVDFHWNSIDPWTLVSVSDDCSSSAGGGTLQIWRIIDLLYRPEEEVLAELDKFRSHVAACSPTPTKDVNHSA

SEQ ID NO: 35 - DNA, 1320 bp, PHYSCOMITRELLA PATENS
  misc_feature (364)..(364): n is a, c, g, or t
  misc_feature (938)..(938): n is a, c, g, or t
atgaagaaggttccgggggatcggtacgggcaatggaagttgcttgtcccagctttgtatgactggctgaccaatcatcaccttatggggccctctctttcttgccggtgggggccgcagttggaggcagggtcctccaagactcgtcaacttcttttttactctgaaaggacggatggagagtgtccaaacaccatcgttgttgcccaatgtgacattatgaagccacgaactgctgcagcggaacagatttcacagttcaaagaaggtggaaaatcacctcacctgaagaagctcaaaactattattcatcctggtgaggttaacagaatacgagaaattcctcagaatagcaatattcttnccacgcatactgatagtcccaaggttctgatatggaataccaaaactcagccaaaccgagctacaacatcagcagcgtctgaatcaaaacccgatctggttttgataggccacactgacaatgctgaattcgctttgaacgtaagtcgaactgctccttacgtcatatcaggaggtaagactcctaaatcaatcacaccgacggcagcagggtccaagcaatcaggaactgcagggggtgctgccgacactactaatgtgtataccagagggatttttaagggccatactgatactgtggaggatgtccaatttcgtccttcgagcatgaatgagttttgcagtgtaggcgatgattcttgcctcctcttgtgggatgctcgcactggataccaaccaatatcgaaggttgtgaaggctcacaatgcggacctccactgtgtcgattggaatgctcatgacgagaatcttattttaactggatcagcagataattctgtaaggttgtttgatcaccgaaagattttagctaggggtcaagctattcccgttgagcaatttgaagggcactccgntgctgtgctctgtgtccagtggtgcccagatagagcttctgtgtttgggagctgcgccgaggatggattgcttaatgtgtgggattatgaaaaggtcggtaaggcattagataccaccaatttgaaacaacctgtaaaagttcctccaggccttttcttccaacacacagggcacagggacaaagtggtggactttcattgggattcgcgcgatccgtggacaatagtcagtgtgtcggaggatgctaacacccccgggggtgggggcactttgcagatttggagaatgatagacttcatttacaggcctgaagacgaagttttggcagaacttgacgaagtgcgacctcaattggtgtctcaaaacacccctgccagttag

SEQ ID NO: 36 - protein, 439 aa, PHYSCOMITRELLA PATENS
  misc_feature (122)..(122): Xaa can be any naturally occurring amino acid
  misc_feature (313)..(313): Xaa can be any naturally occurring amino acid
MKKVPGDRYGQWKLLVPALYDWLTNHHLMGPSLSCRWGPQLEAGSSKTRQLLFYSERTDGECPNTIVVAQCDIMKPRTAAAEQISQFKEGGKSPHLKKLKTIIHPGEVNRIREIPQNSNILXTHTDSPKVLIWNTKTQPNRATTSAASESKPDLVLIGHTDNAEFALNVSRTAPYVISGGKTPKSITPTAAGSKQSGTAGGAADTTNVYTRGIFKGHTDTVEDVQFRPSSMNEFCSVGDDSCLLLWDARTGYQPISKVVKAHNADLHCVDWNAHDENLILTGSADNSVRLFDHRKILARGQAIPVEQFEGHSXAVLCVQWCPDRASVFGSCAEDGLLNVWDYEKVGKALDTTNLKQPVKVPPGLFFQHTGHRDKVVDFHWDSRDPWTIVSVSEDANTPGGGGTLQIWRMIDFIYRPEDEVLAELDEVRPQLVSQNTPAS

SEQ ID NO: 37 - protein, 36 aa, ZEA MAYS
DGSVPNTLVIANCEVVKPRVAAAEHISQFNEEARSP

SEQ ID NO: 38 - protein, 36 aa, SORGHUM BICOLOR; identical to SEQ ID NO: 37

SEQ ID NO: 39 - protein, 36 aa, ZEA MAYS; identical to SEQ ID NO: 37

SEQ ID NO: 40 - protein, 36 aa, GLYCINE MAX; identical to SEQ ID NO: 37

SEQ ID NO: 41 - protein, 36 aa, ARABIDOPSIS THALIANA; identical to SEQ ID NO: 37

SEQ ID NO: 42 - protein, 36 aa, PISUM SATIVUM
DGSVPNTLVIANCEVVKTRVAAAEHISQFNEEARSP

SEQ ID NO: 43 - protein, 36 aa, MEDICAGO TRUNCATULA; identical to SEQ ID NO: 42

SEQ ID NO: 44 - protein, 36 aa, POPULUS
DGSVPNTLVIANCDVVKSRVAAAEHISQFNEEARSP

SEQ ID NO: 45 - protein, 36 aa, GOSSYPIUM HIRSUTUM
DGSVPNTLVIANCEVVKPRVAAAEHISQFNEKTRSP

SEQ ID NO: 46 - protein, 36 aa, PETUNIA HYBRIDA
DGSVPNTLVIANCEVVKPRVAAAEHISQFNEESRSP

SEQ ID NO: 47 - protein, 36 aa, ORYZA SATIVA
DGTVPNTLVIANCEVVKPRVAAAEHISQFNEEARSP

SEQ ID NO: 48 - protein, 36 aa, ORYZA SATIVA; identical to SEQ ID NO: 47

SEQ ID NO: 49 - protein, 36 aa, VITIS VINIFERA
DGSVPNTLVIANCEVVKPRVAAAEHIAQFNEEARSP

SEQ ID NO: 50 - protein, 36 aa, MEDICAGO TRUNCATULA
DGTAPNTLVIATCEIVKPRVAAAEHIAMFNEEARSP

SEQ ID NO: 51 - DNA, 108 bp, PISUM SATIVUM
gatggtagtgtcccaaatactttggtgattgcgaattgtgaggttgtgaaaactagggttgcagctgcagagcatatttcacagtttaatgaggaggctcgctctcca

SEQ ID NO: 52 - DNA, 108 bp, MEDICAGO TRUNCATULA
gatggaagtgttccgaatactttggtgattgcgaattgtgaggttgtgaagactagggttgcagctgctgaacacatttctcagtttaatgaagaggctcgctctcca

SEQ ID NO: 53 - DNA, 108 bp, GLYCINE MAX
gatggtagtgtgccgaatactctggtgattgcgaattgcgaggttgtgaagcctagggttgctgctgctgagcacatttcgcagtttaatgaagaggcgcggtcccca

SEQ ID NO: 54 - DNA, 108 bp, GOSSYPIUM HIRSUTUM
gatggtagtgttccgaatactcttgtgattgctaattgtgaagttgtcaaacctagggttgctgctgctgagcacatatctcagtttaatgaaaaaacacgctcacca

SEQ ID NO: 55 - DNA, 108 bp, POPULUS
gatggtagtgttccaaatactttggtcattgcaaattgtgatgttgtcaagtctagagttgctgctgcggaacacatatctcagtttaatgaagaagcacgctctcca

SEQ ID NO: 56 - DNA, 108 bp, PETUNIA HYBRIDA
gatggaagtgtgccaaacactttggttatagcaaattgtgaagttgttaaacctagggttgcagctgcagagcacatatcacagttcaatgaagaatcacgatctcct

SEQ ID NO: 57 - DNA, 108 bp, ARABIDOPSIS THALIANA
gatggaagtgtgcccaatactttggtcatagcaaattgtgaagttgttaagccaagggttgctgcagcagagcacatttctcagttcaatgaagaagcacgttctcca

SEQ ID NO: 58 - DNA, 108 bp, VITIS VINIFERA
gatggcagtgttccgaataccttagttatagcaaactgtgaagttgttaaacctagggtagctgccgcagagcatattgctcagttcaacgaggaagcgcggtcaccc

SEQ ID NO: 59 - DNA, 108 bp, ZEA MAYS
gatgggagtgtgcctaatactctggttatcgcaaactgtgaagttgtgaaaccaagggttgcagctgctgaacatatctcgcagtttaacgaggaagcacgatcacct

SEQ ID NO: 60 - DNA, 108 bp, SORGHUM BICOLOR
gatgggagtgtgcctaatactctagttatcgcaaactgtgaagttgtgaagccacgggttgcagctgctgaacatatctcgcagtttaacgaggaagcacgatcacct

SEQ ID NO: 61 - DNA, 108 bp, ZEA MAYS
gatgggagtgtgcctaatactctggttatcgcaaattgtgaagttgtgaaacctagggttgcagctgctgaacatatctcacagttcaatgaggaagcacggtcacct

SEQ ID NO: 62 - DNA, 108 bp, ORYZA SATIVA
gatgggactgtccctaatactctggttattgcaaattgtgaagttgtcaaaccaagggttgcagctgctgaacatatatctcagttcaatgaggaagcacgatcacct

SEQ ID NO: 63 - DNA, 108 bp, ORYZA SATIVA; identical to SEQ ID NO: 62

SEQ ID NO: 64 - DNA, 108 bp, MEDICAGO TRUNCATULA
gatggaactgctcccaatacgcttgttattgctacttgtgaaattgttaaacctagggttgctgctgctgaacatattgctatgtttaatgaagaggcgcgttctcca

SEQ ID NO: 65 - protein, 40 aa, GOSSYPIUM HIRSUTUM
IQDHITTMATDPTKSPGSGGSIIKQNKPGEGNDKAADGPS

SEQ ID NO: 66 - protein, 39 aa, POPULUS
IQDHITSSASDPATKSPGSGGSIIKKTGDGSDKATDGPS

SEQ ID NO: 67 - protein, 37 aa, GLYCINE MAX
IEDHITSAATDSKSGGSIIKQNSKSGEGNDKTADGPT

SEQ ID NO: 68 - protein, 36 aa, S: PISUM SATIVUM
IEDHVTSAATDKSGGSIIKPNSKSGEGNDKTVDSPS

SEQ ID NO: 69 - protein, 27 aa, MEDICAGO TRUNCATULA
IEDHITSAATDSNKSGGSIAKTADSPT

SEQ ID NO: 70 - protein, 34 aa, MEDICAGO TRUNCATULA
IHDHIATLATEEEPDVNEGSNVGGNSEKAAQSPS

SEQ ID NO: 71 - protein, 33 aa, VITIS VINIFERA
IQDHISTLAADPGSAKSTSKAGGGNDKPVESPS

SEQ ID NO: 72 - protein, 33 aa, PETUNIA HYBRIDA
IQDHISTLSTDAQKPAGFIKPATTSIKAGDNPS

SEQ ID NO: 73 - protein, 33 aa, ARABIDOPSIS THALIANA
IQDHITTIGTDSKSSGSIIKQTGEGTDKNESPT

SEQ ID NO: 74 - protein, 34 aa, ZEA MAYS
IQDHISALGDSSSSPGASGSKQSIKTANEKESPK

SEQ ID NO: 75 - protein, 34 aa, ZEA MAYS
IQDHISALGDSSSSPGASGSKQSGKIANEKESPK

SEQ ID NO: 76 - protein, 34 aa, SORGHUM BICOLOR
IQDHISALGDSSSSPGASGSKQSGKSATEKESPK

SEQ ID NO: 77 - protein, 35 aa, ORYZA SATIVA
IQDHISALGDSSKTESSPGASGSKGKTANDKDSPK

SEQ ID NO: 78 - protein, 35 aa, ORYZA SATIVA; identical to SEQ ID NO: 77

SEQ ID NO: 79 - DNA, 102 bp, MEDICAGO TRUNCATULA
attcatgatcatattgcaactttagctacagaagaagaacccgatgttaatgagggctctaatgttgggggaaatagcgagaaagctgcacaaagcccgtct

SEQ ID NO: 80 - DNA, 99 bp, VITIS VINIFERA
attcaggatcacatctcgactttggcagcagatccagggtcagcaaagagtacctctaaggctggtggtggtaatgataaacctgtagaaagcccttct

SEQ ID NO: 81 - DNA, 108 bp, PISUM SATIVUM
attgaagaccatgtaacatctgctgctacggacaagtctggtggatccattatcaaaccgaactctaaatctggggaaggcaatgacaaaactgttgatagcccttct

SEQ ID NO: 82 - DNA, 120 bp, GOSSYPIUM HIRSUTUM
atccaggaccacataacaacaatggctacagaccctactaaatctcctggatctggcggatcaatcatcaaacaaaacaagcctggggaaggtaatgacaaagctgctgatgggccttct

SEQ ID NO: 83 - DNA, 117 bp, POPULUS
atccaggaccatataacatcatctgcctctgatccagcaactaagtctccaggatctggtggatcaatcattaaaaagactggggatggcagtgataaagccactgatggcccttct

SEQ ID NO: 84 - DNA, 111 bp, GLYCINE MAX
attgaagaccatataacatctgctgccacagactccaaatctggtgggtcaattatcaaacaaaactctaaatctggagaaggcaatgataaaactgctgatggccctact

SEQ ID NO: 85 - DNA, 81 bp, MEDICAGO TRUNCATULA
attgaagaccatataacatctgctgctacagactccaacaagtctggtggatccattgccaaaactgctgatagccctact

SEQ ID NO: 86 - DNA, 99 bp, PETUNIA HYBRIDA
attcaagatcatatatcaacattgagcacagatgcacaaaaacctgctggtttcatcaagccagccactacatctattaaggctggtgataatccctct

SEQ ID NO: 87 - DNA, 99 bp, ARABIDOPSIS THALIANA
atccaagatcacatcacaacgattgggacagattccaaatcatctggatctatcatcaaacagactggtgaaggtactgataagaatgagagtcctact

SEQ ID NO: 88 - DNA, 102 bp, ZEA MAYS
atccaagaccacatatctgcccttggggattcctcgtcttctcccggagcatctggcagcaagcagtctggcaaaattgcaaatgaaaaggagagtcctaaa

SEQ ID NO: 89 - DNA, 102 bp, SORGHUM BICOLOR
atccaagaccacatatctgcccttggggattcctcgtcttctcctggagcatctggcagcaagcagtctggtaaatctgcaactgaaaaggagagccctaaa

SEQ ID NO: 90 - DNA, 102 bp, ZEA MAYS
atccaagaccatatatctgcccttggggattcctcgtcttctcctggagcatctggcagcaagcagtctattaaaactgcaaatgaaaaggagagccctaaa

SEQ ID NO: 91 - DNA, 105 bp, ORYZA SATIVA
atccaagaccacatatctgcactgggagattcctcgaaaactgaatcttctccaggggcatcaggaagcaagggaaaaactgcaaatgataaggatagtcctaaa

SEQ ID NO: 92 - DNA, 105 bp, ORYZA SATIVA; identical to SEQ ID NO: 91

SEQ ID NO: 93 - protein, 40 aa, GLYCINE MAX
SRPDLILTGHQDNAEFALAMCPTEPYVLSGGKDKTVVLWS

SEQ ID NO: 94 - protein, 40 aa, PISUM SATIVUM; identical to SEQ ID NO: 93

SEQ ID NO: 95 - protein, 40 aa, MEDICAGO TRUNCATULA
SRPDLILTGHQDNAEFALAMCPTQPYVLSGGKDKTVVLWS

SEQ ID NO: 96 - protein, 40 aa, POPULUS
SRPDLILTGHQDNAEFALAMCPTDPYVLSGGKDKFVVLWS

SEQ ID NO: 97 - protein, 40 aa, GOSSYPIUM HIRSUTUM
SRPDLILTGHQDNAEFALAMCPTEPYVLSGGKDKSVVLWS

SEQ ID NO: 98 - protein, 40 aa, ZEA MAYS
SRPDLILTGHKENAEFALAMCPAEPYVLSGGKDKSVVLWS

SEQ ID NO: 99 - protein, 40 aa, SORGHUM BICOLOR; identical to SEQ ID NO: 98

SEQ ID NO: 100 - protein, 40 aa, ORYZA SATIVA
SRPDLILRGHKDIAEFALAMCPAEPYVLSGGKDKSVVWWS

SEQ ID NO: 101 - protein, 40 aa, ORYZA SATIVA; identical to SEQ ID NO: 100

SEQ ID NO: 102 - protein, 40 aa, ZEA MAYS
SRPDLILTGHQENAEFALAMCPAEPYVLSGGKDKFVVLWS

SEQ ID NO: 103 - protein, 40 aa, ARABIDOPSIS THALIANA
SRPDLILTGHQDNAEFALAMCPTEPFVLSGGKDKSVVLWS

SEQ ID NO: 104 - protein, 40 aa, PETUNIA HYBRIDA
SRPDLTLIGHSENAEFALAMCPTEPFVLSGGKDKSVVLWS

SEQ ID NO: 105 - protein, 40 aa, VITIS VINIFERA
KVLFLILTGHKDNAEFALAMCPTEPLVLSGGKDKSVVLWS

SEQ ID NO: 106 - protein, 40 aa, MEDICAGO TRUNCATULA
SIPDLVLTGHKDNAEFALAMCSTEPFVLSGGRDKLVVLWS

SEQ ID NO: 107 - DNA, 120 bp, PISUM SATIVUM
tctcgtccagatttgatattgaccggacaccaagacaatgctgagtttgctcttgcgatgtgcccaactgagccttatgtcctttcaggaggaaaagataaaacagtggtgttgtggagt

SEQ ID NO: 108 - DNA, 120 bp, MEDICAGO TRUNCATULA
tctcgtccagatctgatattgactggacaccaagataatgcagagtttgctcttgcaatgtgccctactcagccctatgtgctttctggaggaaaagacaaaacagtcgtgttgtggagt

SEQ ID NO: 109 - DNA, 120 bp, GLYCINE MAX
tctcgtcctgatttgatattgactggacaccaagataatgcggaatttgctcttgcaatgtgcccaactgaaccctatgttctttcaggaggaaaggacaaaacagtggtgttgtggagt

SEQ ID NO: 110 - DNA, 120 bp, GOSSYPIUM HIRSUTUM
tctcgcccagatttgattttgactgggcatcaagataatgctgaatttgctcttgcaatgtgtccaactgagccttatgtgctctctggagggaaggacaaatcagtggttttgtggagc

SEQ ID NO: 111 - DNA, 120 bp, POPULUS
tctcgtccagatttgattttgactggacatcaagacaatgctgagtttgcccttgcaatgtgcccaactgatccctatgtgctttctggagggaaggacaagttcgtagttttgtggagt

SEQ ID NO: 112 - DNA, 120 bp, SORGHUM BICOLOR
tctcgccctgatctgatattaacgggacataaggaaaatgcggaatttgcgcttgctatgtgcccagcagaaccatatgtcctatcaggaggaaaggacaaatctgttgtcttgtggagc

SEQ ID NO: 113 - DNA, 120 bp, ZEA MAYS
tctcgccctgatctgatattaacaggacataaggaaaatgcggaatttgcgcttgccatgtgtccagcagaaccatatgtcctatcaggaggaaaggacaaatctgttgtcttgtggagc

SEQ ID NO: 114 - DNA, 120 bp, ZEA MAYS
tctcgtcctgatctgatattaacgggacaccaggaaaatgcagaatttgcgcttgccatgtgtccagcagaaccatatgtcctgtcaggaggaaaggacaaatttgttgtcttgtggagc

SEQ ID NO: 115 - DNA, 120 bp, ORYZA SATIVA
tctcgtcctgatttgatattaagaggacataaggatattgctgagtttgcgcttgctatgtgcccagctgagccatatgtgttatctggaggaaaagacaaatctgttgtatggtggagc

SEQ ID NO: 116 - DNA, 120 bp, ORYZA SATIVA
tctcgccctgatttgatattaagaggacataaggatattgctgagtttgcgcttgctatgtgcccagctgagccatatgtgttatctggaggaaaagacaaatctgttgtatggtggagc

SEQ ID NO: 117 - DNA, 120 bp, PETUNIA HYBRIDA
tcacgcccagatttgacattgattggacatagtgagaatgcagaatttgcactggcaatgtgccccactgaaccctttgtgctctctggaggaaaggacaaatctgtggtactgtggagt

SEQ ID NO: 118 - DNA, 120 bp, ARABIDOPSIS THALIANA
tcccgtccagatttgatactaactgggcaccaagataatgctgaatttgctcttgccatgtgcccaacggaaccctttgtgctctccggaggcaaggacaagtcagttgttttgtggagt

SEQ ID NO: 119 - DNA, 120 bp, MEDICAGO TRUNCATULA
tccatcccagacttggtattaactggacataaggataatgctgaatttgcactagctatgtgttcaactgagccctttgttctttccggagggagagacaagcttgtggtgttatggagt

SEQ ID NO: 120 - DNA, 120 bp, VITIS VINIFERA
aaggttttgttcctgatattaactgggcataaagataatgcagaatttgctcttgccatgtgtccaactgaaccattggtgctctctggaggcaaggataagtctgtggtgttgtggagt

SEQ ID NO: 121 - protein, 40 aa, POPULUS
FVKKYKTIIHPGEVNRIRELPQNSKIVATHTDSPDVLIWD

SEQ ID NO: 122 - protein, 40 aa, ARABIDOPSIS THALIANA; identical to SEQ ID NO: 121

SEQ ID NO: 123 - protein, 40 aa, PISUM SATIVUM; identical to SEQ ID NO: 121

SEQ ID NO: 124 - protein, 40 aa, MEDICAGO TRUNCATULA; identical to SEQ ID NO: 121

SEQ ID NO: 125 - protein, 40 aa, ZEA MAYS
FVKKYKTIVHPGEVNRIRELPQNSKIIATHTDSPDVLIWD

SEQ ID NO: 126 - protein, 40 aa, SORGHUM BICOLOR; identical to SEQ ID NO: 125

SEQ ID NO: 127 - protein, 40 aa, ZEA MAYS
FVKKYKTIVHPGEVNRIRELPQNSKIIATHTDSPDVLVWD

SEQ ID NO: 128 - protein, 40 aa, ORYZA SATIVA
FVKKYKTIIHPGEVNRIRELPQNSKIIATHTDSPDVLIWD

SEQ ID NO: 129 - protein, 40 aa, ORYZA SATIVA; identical to SEQ ID NO: 128

SEQ ID NO: 130 - protein, 40 aa, GLYCINE MAX
FVKKYKTIIHPGEVNRIRELPQNSKIVATHTDSPDVLVWD

SEQ ID NO: 131 - protein, 40 aa, VITIS VINIFERA
FVKKFKTIIHPGEVNRIRELPQNSKIVATHTDSPDVLIWD

SEQ ID NO: 132 - protein, 40 aa, GOSSYPIUM HIRSUTUM
FVKKYKTIIHPGEVNRIKELPQSSRIVATHTDSPDVLIWD

SEQ ID NO: 133 - protein, 40 aa, PETUNIA HYBRIDA
FVKKYKTIIHPGEVNRIRELPQNKNIVATHTDSPEVLIWD

SEQ ID NO: 134 - protein, 40 aa, MEDICAGO TRUNCATULA
FVKKVKTILHPGEVNRIRELPSNTNIVATHTDSPNVMIWN

SEQ ID NO: 135 - DNA, 120 bp, PISUM SATIVUM
tttgttaagaagtacaagaccatcatacatcccggggaggtgaacagaattagggaattgccgcaaaattctaagatagtggctactcacacagacagccctgatgttctcatttgggat

SEQ ID NO: 136 - DNA, 120 bp, MEDICAGO TRUNCATULA
tttgtcaagaagtacaagactatcatacatccaggcgaggtgaacagaattagggaattgccacaaaattctaagattgtggccactcacacagacagccctgatgttctcatttgggat

SEQ ID NO: 137 - DNA, 120 bp, GLYCINE MAX
tttgtgaagaagtacaagaccatcatacatcctggtgaggtaaacagaattagggaattgccacaaaattccaagatagtggctacacatacagacagccctgatgtccttgtttgggat

SEQ ID NO: 138 - DNA, 120 bp, POPULUS
tttgttaagaagtacaagaccatcatacatcctggagaggtaaacagaatcagagaactcccccagaatagtaagatagtggctactcatactgacagccctgatgttcttatatgggat

SEQ ID NO: 139 - DNA, 120 bp, GOSSYPIUM HIRSUTUM
tttgtgaagaagtacaaaaccatcatacatcctggagaagtcaacagaatcaaggaacttccacagagctctaggattgtggcaactcacactgatagtcctgatgttcttatttgggat

SEQ ID NO: 140 - DNA, 120 bp, ARABIDOPSIS THALIANA
tttgtgaagaagtacaagaccatcattcaccctggagaggttaacagaatcagggaactcccacagaatagtaagattgttgctactcacaccgacagtcctgatgttctcatttgggat

SEQ ID NO: 141 - DNA, 120 bp, ZEA MAYS
tttgtaaagaagtacaagactatagttcatcctggtgaagttaacagaatcagggagcttccacagaacagtaagatcatagccacacacactgacagtccagatgtacttgtttgggat

SEQ ID NO: 142 - DNA, 120 bp, SORGHUM BICOLOR
tttgttaagaagtacaagaccatagttcatcctggtgaagttaacagaatcagggagcttccacagaacagtaagatcatagccactcacactgacagtccagatgtacttatttgggat

SEQ ID NO: 143 - DNA, 120 bp, ZEA MAYS
tttgtaaagaagtacaaaactatagttcatcctggtgaggttaacagaatcagggaacttccacagaacagtaagatcatagccactcacactgacagtccagatgtacttatttgggat

SEQ ID NO: 144 - DNA, 120 bp, ORYZA SATIVA
tttgtaaagaagtacaagactataattcatccaggcgaggtgaacaggattagggagcttccgcagaacagtaagatcattgccactcataccgacagcccagatgttctcatttgggat

SEQ ID NO: 145 - DNA, 120 bp, ORYZA SATIVA; identical to SEQ ID NO: 144

SEQ ID NO: 146 - DNA, 120 bp, VITIS VINIFERA
tttgtaaagaagttcaaaacaattatacatccaggagaggtgaaccgaatcagggaactgccacagaatagtaagatagtggccacacacactgacagtcctgatgtcctcatttgggat

SEQ ID NO: 147 - DNA, 120 bp, PETUNIA HYBRIDA
tttgtcaagaagtacaaaaccattatacacccaggggaggttaacagaatcagagagcttcctcaaaataaaaacatagtggcaacccatactgatagtcctgaagttctaatttgggat

SEQ ID NO: 148 - DNA, 120 bp, MEDICAGO TRUNCATULA
tttgttaagaaggttaaaactattcttcatcccggtgaggtaaatagaatcagagaactcccgtcaaatactaatatagttgccacacatacagatagtccaaatgttatgatttggaat

SEQ ID NO: 149 - protein, 40 aa, VITIS VINIFERA
IGARGIYQGHDDTVEDVQFCPLSAQEFCSVGDDSCLILWD

SEQ ID NO: 150 - protein, 40 aa, PETUNIA HYBRIDA
IQARGIFQGHEDTVEDVQFCPSSSQEFCSVGDDSCLILWD

SEQ ID NO: 151 - protein, 40 aa, PISUM SATIVUM
VGPRGIYSGHDDTVEDVAFCPSSAQEFCSVGDDSCLILWD

SEQ ID NO: 152 - protein, 40 aa, MEDICAGO TRUNCATULA
VGPRGIYSGHEDTVEDVAFCPSSAQEFCSVGDDSCLILWD

SEQ ID NO: 153 - protein, 40 aa, POPULUS
VGPRGIYQGHEDTVEDVAFCPSSAQEFCSVGDDSCLILWD

SEQ ID NO: 154 - protein, 40 aa, ARABIDOPSIS THALIANA
VGPRGVYHGHEDTVEDVAFSPTSAQEFCSVGDDSCLILWD

SEQ ID NO: 155 - protein, 40 aa, GOSSYPIUM HIRSUTUM
LGPRGVFCGHEDTVEDVTFCPSSAQEFCSVGDDSCLILWD

SEQ ID NO: 156 - protein, 40 aa, GLYCINE MAX
VGPRGIYCGHEDTVEDVTFCPSSAQEFCSVGDDSCLILWD

SEQ ID NO: 157 - protein, 40 aa, MEDICAGO TRUNCATULA
VGARGVYRGHKDTVEDVQFCPSSAQEFCSVGDDSCLILWD

SEQ ID NO: 158 - protein, 40 aa, ZEA MAYS
VDPRGIFHGHDSTVEDVQFCPSSAQEFCSVGDDACLILWD

SEQ ID NO: 159 - protein, 40 aa, ZEA MAYS; identical to SEQ ID NO: 158

SEQ ID NO: 160 - protein, 40 aa, ORYZA SATIVA
VDPRGIFLGHDSTVEDVQFCPSSAQEFCSVGDDSCLILWD

SEQ ID NO: 161 - protein, 40 aa, ORYZA SATIVA; identical to SEQ ID NO: 160

SEQ ID NO: 162 - protein, 9 aa, SORGHUM BICOLOR
VDPRGIFCG

SEQ ID NO: 163 - DNA, 120 bp, PISUM SATIVUM
gtcgggccaagaggtatctactctgggcacgatgatactgttgaagatgtggccttttgcccttctagtgcgcaggaattctgtagtgttggagatgattcttgtctcatattatgggat

SEQ ID NO: 164 - DNA, 120 bp, MEDICAGO TRUNCATULA
gtcggaccaagaggtatctactctgggcatgaggatactgttgaagacgtggctttttgcccttctagtgcgcaggagttctgtagtgttggagatgattcttgtctcatattatgggat

SEQ ID NO: 165 - DNA, 120 bp, GLYCINE MAX
gttggaccacgaggcatttattgtgggcatgaggatacagttgaagatgtgactttctgcccatctagtgcacaggagttctgtagtgttggagatgattcttgtctcatcttatgggat

SEQ ID NO: 166 - DNA, 120 bp, ZEA MAYS
gttgatcctagaggtatattccatggccatgacagtactgttgaagatgttcagttctgcccttccagtgcgcaggagttttgtagtgtgggtgatgatgcttgtcttattctgtgggat

SEQ ID NO: 167 - DNA, 120 bp, ZEA MAYS
gttgatcctagaggtatatttcatggacatgacagcacggttgaagatgttcagttctgcccttccagtgcgcaggagttttgtagtgtgggtgatgatgcttgtcttattctgtgggat

SEQ ID NO: 168 - DNA, 120 bp, ORYZA SATIVA
gttgatcctcgtggtatctttcttggccacgacagtactgttgaagatgtccagttctgcccttctagtgcacaggagttttgtagtgtaggcgatgattcttgtcttattctttgggat

SEQ ID NO: 169 - DNA, 120 bp, ORYZA SATIVA
gttgatcctcgtggtatctttcttggccacgacagtactgttgaagatgtccagttctgcccttctagtgcacaggagttttgtagtgtgggcgatgattcttgtcttattctttgggat

SEQ ID NO: 170 - DNA, 120 bp, PETUNIA HYBRIDA
attcaagctcgtggaatcttccaaggccatgaggataccgttgaagatgttcagttctgcccatcaagttcacaggaattctgtagtgttggtgatgattcatgtctcattctatgggat

SEQ ID NO: 171 - DNA, 120 bp, GOSSYPIUM HIRSUTUM
ttaggtcctcgtggtgtcttctgtgggcatgaggatacagttgaagatgttacattctgtccatcaagtgcacaagagttttgtagtgtaggtgatgattcctgcctcatactatgggat

SEQ ID NO: 172 - DNA, 120 bp, POPULUS
gttggaccgcgaggtatctaccaagggcatgaggatacagttgaagacgtggcattctgtccatccagtgcgcaggagttttgtagtgtaggagatgattcttgccttatattatgggat

SEQ ID NO: 173 - DNA, 120 bp, VITIS VINIFERA
ataggagcacgtggtatctaccaagggcatgatgatactgttgaagatgtgcaattctgcccattaagcgcacaggagttctgtagcgtaggtgatgattcttgccttatcttatgggat

SEQ ID NO: 174 - DNA, 120 bp, ARABIDOPSIS THALIANA
gttggcccacgaggtgtatatcatggccatgaagatacagttgaagatgtggcattcagcccgacgagtgcacaagaattctgcagtgttggtgatgattcttgccttatactatgggat

SEQ ID NO: 175 - DNA, 120 bp, MEDICAGO TRUNCATULA
gttggagcaaggggcgtctaccggggtcataaagacactgttgaagatgtgcagttttgcccctcaagtgcacaggagttctgtagtgtaggtgatgattcttgtctcatactctgggat

SEQ ID NO: 176 - DNA, 27 bp, SORGHUM BICOLOR
gttgatcctagaggtatattctgtggg

SEQ ID NO: 177 - DNA, 30 bp, ARTIFICIAL SEQUENCE
  Synthetic Primer
tttctagagatggaagtgtgcccaatactt

SEQ ID NO: 178 - DNA, 37 bp, ARTIFICIAL SEQUENCE
  Synthetic Primer
ttggccaagatggcctggagaacgtgcttcttcattg

SEQ ID NO: 179 - DNA, 30 bp, ARTIFICIAL SEQUENCE
  Synthetic Primer
ttgagctcgatggaagtgtgcccaatactt

SEQ ID NO: 180 - DNA, 37 bp, ARTIFICIAL SEQUENCE
  Synthetic Primer
ttggccatctaggcctggagaacgtgcttcttcattg

SEQ ID NO: 181 - DNA, 30 bp, ARTIFICIAL SEQUENCE
  Synthetic Primer
tttctagatttgtgaagaagtacaagacca

SEQ ID NO: 182 - DNA, 37 bp, ARTIFICIAL SEQUENCE
  Synthetic Primer
ttggccaagatggccatcccaaatgagaacatcagga

SEQ ID NO: 183 - DNA, 30 bp, ARTIFICIAL SEQUENCE
  Synthetic Primer
ttgagctctttgtgaagaagtacaagacca

SEQ ID NO: 184 - DNA, 37 bp, ARTIFICIAL SEQUENCE
  Synthetic Primer
ttggccatctaggccatcccaaatgagaacatcagga

SEQ ID NO: 185 - DNA, 30 bp, ARTIFICIAL SEQUENCE
  Synthetic Primer
tttctagatcccgtccagatttgatactaa

SEQ ID NO: 186 - DNA, 37 bp, ARTIFICIAL SEQUENCE
  Synthetic Primer
ttggccaagatggccactccacaaaacaactgacttg

SEQ ID NO: 187 - DNA, 30 bp, ARTIFICIAL SEQUENCE
  Synthetic Primer
ttgagctctcccgtccagatttgatactaa

SEQ ID NO: 188 - DNA, 37 bp, ARTIFICIAL SEQUENCE
  Synthetic Primer
ttggccatctaggccactccacaaaacaactgacttg

SEQ ID NO: 189 - DNA, 30 bp, ARTIFICIAL SEQUENCE
  Synthetic Primer
tttctagaatccaagatcacatcacaacga

SEQ ID NO: 190 - DNA, 37 bp, ARTIFICIAL SEQUENCE
  Synthetic Primer
ttggccaagatggccagtaggactctcattcttatca

SEQ ID NO: 191 - DNA, 30 bp, ARTIFICIAL SEQUENCE
  Synthetic Primer
ttgagctcatccaagatcacatcacaacga

SEQ ID NO: 192 - DNA, 37 bp, ARTIFICIAL SEQUENCE
  Synthetic Primer
ttggccatctaggccagtaggactctcattcttatca

SEQ ID NO: 193 - DNA, 30 bp, ARTIFICIAL SEQUENCE
  Synthetic Primer
tttctagagttggcccacgaggtgtatatc

SEQ ID NO: 194 - DNA, 37 bp, ARTIFICIAL SEQUENCE
  Synthetic Primer
ttggccaagatggccatcccatagtataaggcaagaa

SEQ ID NO: 195 - DNA, 30 bp, ARTIFICIAL SEQUENCE
  Synthetic Primer
ttgagctcgttggcccacgaggtgtatatc

SEQ ID NO: 196 - DNA, 37 bp, ARTIFICIAL SEQUENCE
  Synthetic Primer
ttggccatctaggccatcccatagtataaggcaagaa

SEQ ID NO: 197 - DNA, 30 bp, ARTIFICIAL SEQUENCE
  Synthetic Primer
tttctagaatggagagcgacgaagcagcag

SEQ ID NO: 198 - DNA, 30 bp, ARTIFICIAL SEQUENCE
  Synthetic Primer
ttggatccttaaggcttggaggcacaagtc

SEQ ID NO: 199 - DNA, 30 bp, ARTIFICIAL SEQUENCE
  Synthetic Primer
ttggatccatggagagcgacgaagcagcag

SEQ ID NO: 200 - DNA, 30 bp, ARTIFICIAL SEQUENCE
  Synthetic Primer
ttgtcgacttaaggcttggaggcacaagtc

SEQ ID NO: 201 - DNA, 23 bp, ARTIFICIAL SEQUENCE
  Synthetic Primer
gaccctcagagaaacggaaaatg

SEQ ID NO: 202 - DNA, 22 bp, ARTIFICIAL SEQUENCE
  Synthetic Primer
cattgaactgagaaatgtgctc

SEQ ID NO: 203 - DNA, 22 bp, ARTIFICIAL SEQUENCE
  Synthetic Primer
agtgcacaagaattctgcagtg

SEQ ID NO: 204 - DNA, 30 bp, ARTIFICIAL SEQUENCE
  Synthetic Primer
tttctagaatggagagcgacggagcggcag

SEQ ID NO: 205 - DNA, 30 bp, ARTIFICIAL SEQUENCE
  Synthetic Primer
ttgagctctcaaggcttggaggcacatgtc

SEQ ID NO: 206 - DNA, 1524 bp, BRASSICA NAPUS
atggagagcgacggagcggcagcggcaggagcagcgtcgccgagcggaggaaacgtgacaccggcgccgaagaagagaggacggaaaccaaagagcaaggacgagtctcagacgcagcaggagcaggaggaaaggggtgggaaaatgaaggaaagcgggaagaaaacgaagcagggggagcagagcgtggacgagaagtacactcagtggaaaggtctcgtccccatcctctacgactggctcgccaatcacaacctcgtctggccttccctctcttgcagatggggtccacagatcgaacaagcaacctacaagaatcgacagcgtctttacctctcagaacaaactgatggcagtgtgcccaacactctagtcatagcgaattgcgaagttgttaagccaagggtcgctgcagcagagcacatatctcagttcaacgaagaagcacgttctccatttgtgaagaagttcaagaccatcattcaccctggagaggttaaccgaatcagggaactcccacaaaacagtaagattattgctactcacaccgacagtcctgatgttctcatttgggatgttgaaacccaaccaaaccgtcatgctgtccttggagctgcacattcccgtcctgatttggtattaactggacatcaagacaatgctgaattcgctcttgcaatgtgcccaaccgaaccctttgtcctctctggaggcaaagacaagtcagttgttttgtggagtatccaggaccacatcacaaccgctggtagtacagactccaaatcatctggatccatcatcaaacagactggtgaaggtggtgataagactgagagtccttctcttggcccgcgaggtgtatatcatggccatgatgataccgttgaagatgtcgctttcagccccactagtgcacaagagttctgcagtgtcggtgacgattcttgccttatactatgggatgcaagaacaggcactagccctgtcacgaaggttgaaaaggcgcacgatgctgatcttcattgtgtcgattggaaccctcatgatgacaatctgatcctgacagggtctgcagacaacactgttcggttgtatgatcgtaggaaactaacctcaaatggagtgggtacgcctatttacaaatttgaaggccacaaagctgctgttctttgcgttcagtggtctcctgataagtcatctgtttttgggagttccgcggaagatggtctcttgaacatctgggattatgacagggtcagtaagaagtctgatcgtgcagctaaaagtccggctggtctcttcttccagcatgctggtcacagggacaaagttgttgatttccactggaatgcagaggacccttggactattgtcagtgtttctgatgactgcgagactactggtggaggtggaacactgcagatatggcggatgagtgacttgatttacagaccggaggaggaagttctggcagagttggagaagttcaagtcgcatgttatgacatgtgcctccaagccttga

SEQ ID NO: 207 - protein, 507 aa, BRASSICA NAPUS
MESDGAAAAGAASPSGGNVTPAPKKRGRKPKSKDESQTQQEQEERGGKMKESGKKTKQGEQSVDEKYTQWKGLVPILYDWLANHNLVWPSLSCRWGPQIEQATYKNRQRLYLSEQTDGSVPNTLVIANCEVVKPRVAAAEHISQFNEEARSPFVKKFKTIIHPGEVNRIRELPQNSKIIATHTDSPDVLIWDVETQPNRHAVLGAAHSRPDLVLTGHQDNAEFALAMCPTEPFVLSGGKDKSVVLWSIQDHITTAGSTDSKSSGSIIKQTGEGGDKTESPSLGPRGVYHGHDDTVEDVAFSPTSAQEFCSVGDDSCLILWDARTGTSPVTKVEKAHDADLHCVDWNPHDDNLILTGSADNTVRLYDRRKLTSNGVGTPIYKFEGHKAAVLCVQWSPDKSSVFGSSAEDGLLNIWDYDRVSKKSDRAAKSPAGLFFQHAGHRDKVVDFHWNAEDPWTIVSVSDDCETTGGGGTLQIWRMSDLIYRPEEEVLAELEKFKSHVMTCASKP

SEQ ID NO: 208 - DNA, 31 bp, ARTIFICIAL SEQUENCE
  Synthetic Primer
tttctagaatgaaagagagaggcggctccag

SEQ ID NO: 209 - DNA, 31 bp, ARTIFICIAL SEQUENCE
  Synthetic Primer
ttgagctctcaattcttcggtgcacagctgg

SEQ ID NO: 210 - DNA, 31 bp, ARTIFICIAL SEQUENCE
  Synthetic Primer
tttctagaatgaaggagagaggcggctccag

SEQ ID NO: 211 - DNA, 31 bp, ARTIFICIAL SEQUENCE
  Synthetic Primer
ttgagctctcaattcctcggagcgcaactgg

SEQ ID NO: 212 - DNA, 21 bp, ARTIFICIAL SEQUENCE
  Synthetic Primer
atatgttcagcagctgcaacc

SEQ ID NO: 213 - DNA, 21 bp, ARTIFICIAL SEQUENCE
  Synthetic Primer
gctgctgttctttgtgttcag

SEQ ID NO: 214 - DNA, 31 bp, ARTIFICIAL SEQUENCE
  Synthetic Primer
tttctagaatgaaggagaggagcggctcgag

SEQ ID NO: 215 - DNA, 30 bp, ARTIFICIAL SEQUENCE
  Synthetic Primer
ttgagctctcagttcctcggagtgcagctg

SEQ ID NO: 216 - DNA, 1362 bp, PANICUM VIRGATUM
atgaaggagaggagcggctcgagggcggcggtggatgagcgctacgcgcagtggaagtcgctcattccggtgctctacgactggttcgccaaccacaacctcgtctggccgtcgctgtcctgccggtggggcccgcaatttgagaaagctacctacaagaatcgccagcgcctttacctatctgagcagacggatgggagtgtgcctaatactctggttattgcgaactgtgaagttgtcaaaccaagggttgcagctgctgaacatatctcacagttcaatgaggaagcacgatcgccttttgtgaagaagtataagactatagttcatcctggtgaggttaacagaatcagggagcttccacagaacagtaagatcatagccactcacaccgacagtccagatgtacttatttgggatgttgaagcccaaccaaatagacatgctgtcctaggagcaactgaatctcggcctgatctgatattaacaggacatcaggaaaatgctgaattcgcgcttgccatgtgtccagcagaaccatatgtactgtcaggaggaaaggacaaatctgttgtcttgtggagcatccaagaccacatatctgcccttggggattcctcatcttctcctggagcatctggcagcaagcagtctggcaaaactgcgaatgaaaaggagagtcccaaagttgatcctcggggtatattccatggacatgacagcactgttgaagatgttcagttctgcccttccagtgcacaggaattctgtagtgtgggtgatgatgcttgtcttattctctgggatgcccggactggtacgagcccggctgttaaggttgagaaagctcatagtggggatgttcattgtgtggattggaatccgcttgatgttaactatatcttaactggttctgctgataactctgtccgtatgtgggatcgtcgcaatctgggttctggaggagctggttctccaattcacaaatttgagggccataaagctgctgttctttgtgtccagtggtcacctgacagagcatctgttttcggaagttctgcggaagatggtttcttaaatgtgtgggatcatgagaaggttgggaagaaaaaaaattctaacgtaccagctgggcttttctttcaacatgctggtcacagggataagattgtagacttccactggaattcatcggatccttggacaatcgtcagtgtctcagatgatggtgagagcaccggtggaggtggaacactgcagatatggcggatgagtgatttgatctaccgcccagaggaggaagttctccaagaactggagaatttcaaggctcacttggccagctgcactccgaggaactga

SEQ ID NO: 217 - protein, 453 aa, PANICUM VIRGATUM
MKERSGSRAAVDERYAQWKSLIPVLYDWFANHNLVWPSLSCRWGPQFEKATYKNRQRLYLSEQTDGSVPNTLVIANCEVVKPRVAAAEHISQFNEEARSPFVKKYKTIVHPGEVNRIRELPQNSKIIATHTDSPDVLIWDVEAQPNRHAVLGATESRPDLILTGHQENAEFALAMCPAEPYVLSGGKDKSVVLWSIQDHISALGDSSSSPGASGSKQSGKTANEKESPKVDPRGIFHGHDSTVEDVQFCPSSAQEFCSVGDDACLILWDARTGTSPAVKVEKAHSGDVHCVDWNPLDVNYILTGSADNSVRMWDRRNLGSGGAGSPIHKFEGHKAAVLCVQWSPDRASVFGSSAEDGFLNVWDHEKVGKKKNSNVPAGLFFQHAGHRDKIVDFHWNSSDPWTIVSVSDDGESTGGGGTLQIWRMSDLIYRPEEEVLQELENFKAHLASCTPRN

SEQ ID NO: 218 - DNA, 30 bp, ARTIFICIAL SEQUENCE
  Synthetic Primer
tttctagaatggagactcctcctccccaac

SEQ ID NO: 219 - DNA, 31 bp, ARTIFICIAL SEQUENCE
  Synthetic Primer
ttgagctctcatttttcagtctttgaagcac

SEQ ID NO: 220 - DNA, 1362 bp, ZEA MAYS
atgaaagagagaggcggctccagggcggcggtggacgagcgctacgcgcagtggaagtcgctcatccccgtcctctacgactggtttgccaaccacaacctcgtctggccatccctctcctgccggtgggggccacagtttgagaaagctacctacaagaatcgtcagcgcctgtacctatctgaacagacggatgggagtgtgcctaatactctggttatcgcaaattgtgaagttgtgaaacctagggttgcagctgctgaacatatctcacagttcaatgaggaagcacggtcaccttttgtaaagaagtacaaaactatagttcatcctggtgaggttaacagaatcagggaacttccacagaacagtaagatcatagccactcacactgacagtccagatgtacttatttgggatgttgaagcacaaccaaatagacatgctgtcctaggagcaagcgagtctcgccctgatctgatattaacaggacataaggaaaatgcggaatttgcgcttgccatgtgtccagcagaaccatatgtcctatcaggaggaaaggacaaatctgttgtcttgtggagcatccaagaccatatatctgcccttggggattcctcgtcttctcctggagcatctggcagcaagcagtctattaaaactgcaaatgaaaaggagagccctaaagttgatcctagaggtatatttcatggacatgacagcacggttgaagatgttcagttctgcccttccagtgcgcaggagttttgtagtgtgggtgatgatgcttgtcttattctgtgggatgctcgaactggcactgccccagctgttaaggttgagaaagctcacagtggagatgttcattgtgttgattggaatccccttgatgttaactatatcttaactgggtctgccgataactctgtccgaatgtgggatcgtcggaatctgggttcaggaggagctggttctccaattcacaaatttgaaggccataaagctgctgttctttgtgttcagtggtcacctgacagagcatctgtttttggaagttctgcagaagatggtttcttaaacgtgtgggatcatgagaaggtggggaagaagaaaaattctaatgtcccagccgggcttttctttcagcacgctggtcatagggataagattgtagacttccactggaattcgtcagatccttggacaattgtcagtgtctctgatgatggcgagagcactggtggaggtggaacactgcagatttggcgcatgagtgatttgatctaccgcccagaggatgaagttcttgcagagctggagaatttcaaggctcatttggccagctgtgcaccgaagaattga

SEQ ID NO: 221 - protein, 453 aa, ZEA MAYS
MKERGGSRAAVDERYAQWKSLIPVLYDWFANHNLVWPSLSCRWGPQFEKATYKNRQRLYLSEQTDGSVPNTLVIANCEVVKPRVAAAEHISQFNEEARSPFVKKYKTIVHPGEVNRIRELPQNSKIIATHTDSPDVLIWDVEAQPNRHAVLGASESRPDLILTGHKENAEFALAMCPAEPYVLSGGKDKSVVLWSIQDHISALGDSSSSPGASGSKQSIKTANEKESPKVDPRGIFHGHDSTVEDVQFCPSSAQEFCSVGDDACLILWDARTGTAPAVKVEKAHSGDVHCVDWNPLDVNYILTGSADNSVRMWDRRNLGSGGAGSPIHKFEGHKAAVLCVQWSPDRASVFGSSAEDGFLNVWDHEKVGKKKNSNVPAGLFFQHAGHRDKIVDFHWNSSDPWTIVSVSDDGESTGGGGTLQIWRMSDLIYRPEDEVLAELENFKAHLASCAPKN

SEQ ID NO: 222 - skipped

SEQ ID NO: 223 - protein, 34 aa, BRASSICA NAPUS
IQDHITTAGSTDSKSSGSIIKQTGEGGDKTESPS

SEQ ID NO: 224 - DNA, 30 bp, ARTIFICIAL SEQUENCE
  Synthetic Primer
tttctagaatccaggaccacatcacaaccg

SEQ ID NO: 225 - DNA, 37 bp, ARTIFICIAL SEQUENCE
  Synthetic Primer
ttggccaagatggccagaaggactctcagtcttatca

SEQ ID NO: 226 - DNA, 30 bp, ARTIFICIAL SEQUENCE
  Synthetic Primer
ttgagctcatccaggaccacatcacaaccg

SEQ ID NO: 227 - DNA, 37 bp, ARTIFICIAL SEQUENCE
  Synthetic Primer
ttggccatctaggccagaaggactctcagtcttatca

SEQ ID NO: 228 - protein, 40 aa, BRASSICA NAPUS
SRPDLVLTGHQDNAEFALAMCPTEPFVLSGGKDKSVVLWS

SEQ ID NO: 229 - DNA, 30 bp, ARTIFICIAL SEQUENCE
  Synthetic Primer
tttctagatcccgtcctgatttggtattaa

SEQ ID NO: 230 - DNA, 37 bp, ARTIFICIAL SEQUENCE; identical to SEQ ID NO: 186
  Synthetic Primer

SEQ ID NO: 231 - DNA, 30 bp, ARTIFICIAL SEQUENCE
  Synthetic Primer
ttgagctctcccgtcctgatttggtattaa

SEQ ID NO: 232 - DNA, 37 bp, ARTIFICIAL SEQUENCE; identical to SEQ ID NO: 188
  Synthetic Primer

SEQ ID NO: 233 - DNA, 102 bp, BRASSICA NAPUS
atccaggaccacatcacaaccgctggtagtacagactccaaatcatctggatccatcatcaaacagactggtgaaggtggtgataagactgagagtccttct

SEQ ID NO: 234 - DNA, 120 bp, BRASSICA NAPUS
tcccgtcctgatttggtattaactggacatcaagacaatgctgaattcgctcttgcaatgtgcccaaccgaaccctttgtcctctctggaggcaaagacaagtcagttgttttgtggagt

SEQ ID NO: 235 - DNA, 120 bp, BRASSICA NAPUS
tttgtgaagaagttcaagaccatcattcaccctggagaggttaaccgaatcagggaactcccacaaaacagtaagattattgctactcacaccgacagtcctgatgttctcatttgggat

SEQ ID NO: 236 - protein, 40 aa, BRASSICA NAPUS
FVKKFKTIIHPGEVNRIRELPQNSKIIATHTDSPDVLIWD

SEQ ID NO: 237 - DNA, 30 bp, ARTIFICIAL SEQUENCE
  Synthetic Primer
tttctagatttgtgaagaagttcaagacca

SEQ ID NO: 238 - DNA, 30 bp, ARTIFICIAL SEQUENCE
  Synthetic Primer
ttgagctctttgtgaagaagttcaagacca

SEQ ID NO: 239 - DNA, 1536 bp, GLYCINE MAX
atggagactcctcctccccaacaaggcgtcgtgaagaagaaggagacaaggggccgaaaacccaaaccaaaggacgaacacgggaagggtttgaaggaaggtaggaaaacacaacaacaacaacaacaacaacatcatcatcagcagcagcaacagcaacaagatcaaccttcggtggacgagaaatacacgcagtggaagtcccttgttcctgtcctctacgactggctcgccaaccacaacctcgtctggccctctctctcttgcaggtggggcccccagcttgaacaagccacttacaagaatcgccagagactctacctttctgagcagactgatggtagtgtgccgaatactctggtgattgcgaattgcgaggttgtgaagcctagggttgctgctgctgagcacatttcgcagtttaatgaagaggcgcggtccccatttgtgaagaagtacaagaccatcatacatcctggtgaggtaaacagaattagggaattgccacaaaattccaagatagtggctacacatacagacagccctgatgtccttgtttgggatgttgaaagtcaacctaatcgccatgctgtccttggagctacaaactctcgtcctgatttgatattgaccggacaccaagataatgcggaatttgctcttgctatgtgcccaactgaaccctatgttctttcaggaggaaaggacaaaacagtggtgttgtggagtattgaagaccatataacatctgctgctacagactccaaatctggtgggtcaattatcaaacaaaactctaaatctggagaaggcaatgacaaaactgctgatggccctactgttggaccacgaggtatctattgtgggcatgaggatactgttgaagacgtggctttctgcccatctagtgcacaggagttctgtagtgttggagatgattcttgtctcatcttatgggatgcacgtgttggctctagccctgtggttaaggttgagaaagctcataatgctgatcttcactgtgtggactggaatccccatgatgataatctgattcttactgggtcagcagataattctgttcgcatgtttgatcgccgcaatctcaccactaatggagttgggtcacccatccataaatttgagggtcacaaagctgctgttctttgtgttcagtggtctccagacaaatcatctgtatttggaagttcagctgaagatggtctcttaaacatttgggactatgagaaggttggtaaaaagatagagcgatctggaaaatcaataagttctcctccagggttgttttttcaacatgcaggtcatagggataaagttgttgacttccattggaatgcatatgatccatggacgattgttagtgtgtctgatgactgtgaaagtactggaggagggggaacgttgcagatatggcgcatgagtgatttgatctacagaccagaagatgaggttttggccgagctggagaaattcaaatctcacgttgtggcgtgtgcttcaaagactgaaaaatga

SEQ ID NO: 240 - protein, 511 aa, GLYCINE MAX
METPPPQQGVVKKKETRGRKPKPKDEHGKGLKEGRKTQQQQQQQHHHQQQQQQQDQPSVDEKYTQWKSLVPVLYDWLANHNLVWPSLSCRWGPQLEQATYKNRQRLYLSEQTDGSVPNTLVIANCEVVKPRVAAAEHISQFNEEARSPFVKKYKTIIHPGEVNRIRELPQNSKIVATHTDSPDVLVWDVESQPNRHAVLGATNSRPDLILTGHQDNAEFALAMCPTEPYVLSGGKDKTVVLWSIEDHITSAATDSKSGGSIIKQNSKSGEGNDKTADGPTVGPRGIYCGHEDTVEDVAFCPSSAQEFCSVGDDSCLILWDARVGSSPVVKVEKAHNADLHCVDWNPHDDNLILTGSADNSVRMFDRRNLTTNGVGSPIHKFEGHKAAVLCVQWSPDKSSVFGSSAEDGLLNIWDYEKVGKKIERSGKSISSPPGLFFQHAGHRDKVVDFHWNAYDPWTIVSVSDDCESTGGGGTLQIWRMSDLIYRPEDEVLAELEKFKSHVVACASKTEK

SEQ ID NO: 241 - DNA, 223 bp, BRACHYPODIUM DISTACHYON
atgaaggagaggagcggctcgagggcggcggtggatgagcgctacgcgcagtggaagtcgctcattccggtgctctacgactggttcgccaaccacaacctcgtctggccgtcgctgtcctgccggtggggcccgcaatttgagaaagctacctacaagaatcgccagcgcctttacctatctgagcagacggatgggagtgtgcctaatactctggttatgc

SEQ ID NO: 242 - protein, 74 aa, BRACHYPODIUM DISTACHYON
MKERSGSRAAVDERYAQWKSLIPVLYDWFANHNLVWPSLSCRWGPQFEKATYKNRQRLYLSEQTDGSVPNTLVM

SEQ ID NO: 243 - protein, 36 aa, BRASSICA NAPUS; identical to SEQ ID NO: 37

SEQ ID NO: 244 - protein, 36 aa, PANICUM VIRGATUM; identical to SEQ ID NO: 37

SEQ ID NO: 245 - DNA, 108 bp, BRASSICA NAPUS
gatggcagtgtgcccaacactctagtcatagcgaattgcgaagttgttaagccaagggtcgctgcagcagagcacatatctcagttcaacgaagaagcacgttctcca

SEQ ID NO: 246 - DNA, 108 bp, PANICUM VIRGATUM
gatgggagtgtgcctaatactctggttattgcgaactgtgaagttgtcaaaccaagggttgcagctgctgaacatatctcacagttcaatgaggaagcacgatcgcct

SEQ ID NO: 247 - protein, 34 aa, PANICUM VIRGATUM
IQDHISALGDSSSSPGASGSKQSGKTANEKESPK

SEQ ID NO: 248 - DNA, 102 bp, PANICUM VIRGATUM
atccaagaccacatatctgcccttggggattcctcatcttctcctggagcatctggcagcaagcagtctggcaaaactgcgaatgaaaaggagagtcccaaa

SEQ ID NO: 249 - protein, 40 aa, PANICUM VIRGATUM; identical to SEQ ID NO: 125

SEQ ID NO: 250 - DNA, 120 bp, PANICUM VIRGATUM
tttgtgaagaagtataagactatagttcatcctggtgaggttaacagaatcagggagcttccacagaacagtaagatcatagccactcacaccgacagtccagatgtacttatttgggat

SEQ ID NO: 251 - protein, 40 aa, PANICUM VIRGATUM
SRPDLILTGHQENAEFALAMCPAEPYVLSGGKDKSVVLWS

SEQ ID NO: 252 - DNA, 120 bp, PANICUM VIRGATUM
tctcggcctgatctgatattaacaggacatcaggaaaatgctgaattcgcgcttgccatgtgtccagcagaaccatatgtactgtcaggaggaaaggacaaatctgttgtcttgtggagc

SEQ ID NO: 253 - protein, 40 aa, PANICUM VIRGATUM; identical to SEQ ID NO: 158

SEQ ID NO: 254 - DNA, 120 bp, PANICUM VIRGATUM
gttgatcctcggggtatattccatggacatgacagcactgttgaagatgttcagttctgcccttccagtgcacaggaattctgtagtgtgggtgatgatgcttgtcttattctctgggat

SEQ ID NO: 255 - protein, 40 aa, BRASSICA NAPUS
LGPRGVYHGHDDTVEDVAFSPTSAQEFCSVGDDSCLILWD

SEQ ID NO: 256 - DNA, 120 bp, BRASSICA NAPUS
cttggcccgcgaggtgtatatcatggccatgatgataccgttgaagatgtcgctttcagccccactagtgcacaagagttctgcagtgtcggtgacgattcttgccttatactatgggat

SEQ ID NO: 257 - protein, 40 aa, MEDICAGO TRUNCATULA
FPAVKVEKAHDGDVHCVDWNTHDINFILTGSADNTVRMFD

SEQ ID NO: 258 - protein, 40 aa, VITIS VINIFERA
TPAIKVEKAHNADLHCVDWNPHDINLILTGSADNTVRMFD

SEQ ID NO: 259 - protein, 40 aa, GOSSYPIUM HIRSUTUM
GPTVKVEKAHNADLHCVDWNPHDDNLILTGSADHTVRMFD

SEQ ID NO: 260 - protein, 40 aa, POPULUS
SPAIKVERAHNADLHCVDWNPQDDNLILTGSADTSVCMFD

SEQ ID NO: 261 - protein, 40 aa, ARABIDOPSIS THALIANA
NPVTKVEKAHDADLHCVDWNPHDDNLILTGSADNTVRLFD

SEQ ID NO: 262 - protein, 40 aa, GLYCINE MAX
SPVVKVEKAHNADLHCVDWNPHDDNLILTGSADNSVRMFD

SEQ ID NO: 263 - protein, 40 aa, PISUM SATIVUM
SPVVKVEKAHNADLHCVDWNPHDDNLILTGSADNSVRLFD

SEQ ID NO: 264 - protein, 40 aa, MEDICAGO TRUNCATULA
SPVVKVEKAHDADLHCVDWNPHDDNLILTGSADNSIRMFD

SEQ ID NO: 265 - protein, 40 aa, PETUNIA HYBRIDA
SPVVKVEKAHNADLHCVDWNPHDGNFIITGSADNSVRLFD

SEQ ID NO: 266 - protein, 40 aa, ZEA MAYS
APAVKVEKAHSGDVHCVDWNPLDVNYILTGSADNSVRMWD

SEQ ID NO: 267 - protein, 40 aa, ZEA MAYS
DPAVKVEKAHSGDVHCVDWNPLDVNYILTGSADNSVRMWD

SEQ ID NO: 268 - protein, 24 aa, SORGHUM BICOLOR
VDWNPLDVNYILTGSADNSVRMWD

SEQ ID NO: 269 - protein, 40 aa, ORYZA SATIVA
GPAVKVEKAHGGDVHCVDWNLHDVNYILTGSADNSVRMWD

SEQ ID NO: 270 - protein, 30 aa, ORYZA SATIVA
GPAVKVEKAHGGDVHCVDWNLHDVNYILTG

SEQ ID NO: 271 - protein, 40 aa, BRASSICA NAPUS
SPVTKVEKAHDADLHCVDWNPHDDNLILTGSADNTVRLYD

SEQ ID NO: 272 - protein, 40 aa, PANICUM VIRGATUM
SPAVKVEKAHSGDVHCVDWNPLDVNYILTGSADNSVRMWD

SEQ ID NO: 273 - DNA, 120 bp, MEDICAGO TRUNCATULA
tttccagctgtcaaggttgagaaggcacatgatggagatgtacattgcgttgattggaatactcatgacatcaattttattctgactggctctgctgataacacagttcgcatgtttgat

SEQ ID NO: 274 - DNA, 120 bp, VITIS VINIFERA
actccagccatcaaggtcgagaaagctcataatgctgatcttcactgtgttgattggaatcctcatgacataaatcttattctaactggatcggctgacaatactgttcgcatgtttgat

SEQ ID NO: 275 - DNA, 120 bp, GOSSYPIUM HIRSUTUM
ggccccactgtcaaggttgaaaaggcacataatgctgatctccattgtgttgattggaacccccatgatgacaatcttatcctgactgggtctgcagatcatactgtacgtatgtttgat

SEQ ID NO: 276 - DNA, 120 bp, POPULUS
agcccagctataaaggttgaaagggcacataatgctgatcttcactgtgttgattggaatcctcaagatgataatctcattttaactgggtctgcagatacttctgtttgcatgtttgat

SEQ ID NO: 277 - DNA, 120 bp, ARABIDOPSIS THALIANA
aaccctgtcacgaaggttgaaaaagcgcatgatgctgatcttcattgtgttgattggaatcctcatgacgacaatctgatcctgacagggtcagcagacaacactgtccggttgtttgat

SEQ ID NO: 278 - DNA, 120 bp, GLYCINE MAX
agccctgtggttaaggttgagaaagctcataatgctgatcttcactgtgtggactggaatccccatgatgataatctgattcttactgggtcagcagataattctgttcgcatgtttgat

SEQ ID NO: 279 - DNA, 120 bp, PISUM SATIVUM
agtcctgttgttaaggttgaaaaagctcataatgccgatcttcactgtgttgactggaatccccatgatgataatctgattcttactgggtcggcagataattctgttcgcttgtttgat

SEQ ID NO: 280 - DNA, 120 bp, MEDICAGO TRUNCATULA
agtcctgtggttaaggttgaaaaagctcatgatgctgatcttcactgtgttgactggaatccccatgatgataatctgattcttactgggtcggcagataattctattcgcatgtttgat

SEQ ID NO: 281 - DNA, 120 bp, PETUNIA HYBRIDA
agtccagttgtaaaggttgaaaaagctcataatgctgatctccactgtgttgactggaatcctcatgatggcaactttattatcactggatctgcagataattctgtccgcttgtttgat

SEQ ID NO: 282 - DNA, 120 bp, ZEA MAYS
gccccagctgttaaggttgagaaagctcacagtggagatgttcattgtgttgattggaatccccttgatgttaactatatcttaactgggtctgccgataactctgtccgaatgtgggat

SEQ ID NO: 283 - DNA, 120 bp, ZEA MAYS
gacccagctgttaaggttgagaaagctcacagtggagatgttcattgtgttgattggaatccccttgatgttaactatatcttaactggttctgccgataactctgtccgaatgtgggat

SEQ ID NO: 284 - DNA, 72 bp, SORGHUM BICOLOR
gttgattggaatccccttgacgttaactatatcttaactggttctgccgataactctgtccgaatgtgggat

SEQ ID NO: 285 - DNA, 120 bp, ORYZA SATIVA
ggcccagctgttaaggttgagaaagctcatggtggtgatgttcattgtgttgactggaacctccatgatgttaactatatcttaactggttctgcggataattctgtccgtatgtgggac

SEQ ID NO: 286 - DNA, 90 bp, ORYZA SATIVA
ggcccagctgttaaggttgagaaagctcatggtggtgatgttcattgtgttgactggaacctccatgatgttaactatatcttaactggt

SEQ ID NO: 287 - DNA, 120 bp, BRASSICA NAPUS
agccctgtcacgaaggttgaaaaggcgcacgatgctgatcttcattgtgtcgattggaaccctcatgatgacaatctgatcctgacagggtctgcagacaacactgttcggttgtatgat

SEQ ID NO: 288 - DNA, 120 bp, PANICUM VIRGATUM
agcccggctgttaaggttgagaaagctcatagtggggatgttcattgtgtggattggaatccgcttgatgttaactatatcttaactggttctgctgataactctgtccgtatgtgggat

SEQ ID NO: 289 - protein, 40 aa, MEDICAGO TRUNCATULA
GSPVYKFEGHDEPVLCVQWNPAKSSVFGSGAEDGIINIWD

SEQ ID NO: 290 - protein, 40 aa, VITIS VINIFERA
GSPIHTFEGHTAAVLCVQWSPDKASIFGSSAEDGILNLWN

SEQ ID NO: 291 - protein, 40 aa, GOSSYPIUM HIRSUTUM
GSPIYKFEGHKAAVLCVQWSPDKSSVFGSSAEDGLLNIWD

SEQ ID NO: 292 - protein, 40 aa, POPULUS
GLPVYKFEGHKAAVLCVQWSPDKASVFGSSAEDGLLNIWD

SEQ ID NO: 293 - protein, 40 aa, ARABIDOPSIS THALIANA; identical to SEQ ID NO: 291

SEQ ID NO: 294 - protein, 40 aa, GLYCINE MAX
GSPIHKFEGHKAAVLCVQWSPDKSSVFGSSAEDGLLNIWD

SEQ ID NO: 295 - protein, 40 aa, PISUM SATIVUM
GSPIHKFEAHKAAVLCVQWSPDKSSVFGSSAEDGLLNIWD

SEQ ID NO: 296 - protein, 40 aa, MEDICAGO TRUNCATULA; identical to SEQ ID NO: 295

SEQ ID NO: 297 - protein, 40 aa, PETUNIA HYBRIDA
GSPVHIFENHKAAVLCVQWCPDRSSVFGSTAEDGRLNIWD

SEQ ID NO: 298 - protein, 40 aa, ZEA MAYS
GSPIHKFEGHKAAVLCVQWSPDRASVFGSSAEDGFLNVWD

SEQ ID NO: 299 - protein, 40 aa, ZEA MAYS; identical to SEQ ID NO: 298

SEQ ID NO: 300 - protein, 40 aa, SORGHUM BICOLOR
SSPIHKFEGHKAAVLCVQWSPDRASVFGSSAEDGFLNVWD

SEQ ID NO: 301 - protein, 40 aa, ORYZA SATIVA
GIPVHKFEGHKAAVLCVQWSPDKASVFGSSAEDGFLNVWD

SEQ ID NO: 302 - protein, 22 aa, ORYZA SATIVA
WSPDKASVFGSSAEDGFLNVWD

SEQ ID NO: 303 - protein, 40 aa, BRASSICA NAPUS
GTPIYKFEGHKAAVLCVQWSPDKSSVFGSSAEDGLLNIWD

SEQ ID NO: 304 - protein, 40 aa, PANICUM VIRGATUM; identical to SEQ ID NO: 298

SEQ ID NO: 305 - DNA, 120 bp, MEDICAGO TRUNCATULA
gggtctcctgtttataaatttgaaggccatgatgaaccagtcctctgtgtacagtggaatcctgctaaatcatctgtatttggaagtggtgccgaagatggaattataaacatctgggac

SEQ ID NO: 306 - DNA, 120 bp, VITIS VINIFERA
ggatcaccaatccatacatttgagggccatactgctgctgtcctttgtgtacagtggtctccggacaaggcttcgatctttgggagttctgcagaagatggtatcttaaacctctggaat

SEQ ID NO: 307 - DNA, 120 bp, GOSSYPIUM HIRSUTUM
ggatcacccatttacaagtttgagggtcataaagctgctgttctatgcgtgcagtggtctccagacaaatcatctgtatttggcagttctgctgaggatgggctcttgaacatttgggac

SEQ ID NO: 308 - DNA, 120 bp, POPULUS
ggtttgccagtctataaatttgagggtcacaaagctgctgttctctgtgtacagtggtctccagataaggcatctgtttttgggagttctgcggaggatggtctcttgaatatttgggat

SEQ ID NO: 309 - DNA, 120 bp, ARABIDOPSIS THALIANA
ggttcgcctatttacaaatttgagggacacaaagctgctgttctttgtgttcagtggtctcctgataagtcatccgtctttgggagctctgcagaagatggtctcttgaacatctgggat

SEQ ID NO: 310 - DNA, 120 bp, GLYCINE MAX
gggtcacccatccataaatttgagggtcacaaagctgctgttctttgtgttcagtggtctccagacaaatcatctgtatttggaagttcagctgaagatggtctcttaaacatttgggac

SEQ ID NO: 311 - DNA, 120 bp, PISUM SATIVUM
gggtctcctattcataaatttgaggctcataaagctgccgttctttgtgttcagtggtctccagacaaatcatctgtatttggaagttcagcagaagacggtctcttaaacatttgggat

SEQ ID NO: 312 - DNA, 120 bp, MEDICAGO TRUNCATULA
gggtctcctatccacaaatttgaggctcataaggctgctgtcctttgtgttcagtggtctccagacaaatcatctgtatttggaagttcagcagaagatggtctcctgaacatttgggat

SEQ ID NO: 313 - DNA, 120 bp, PETUNIA HYBRIDA
ggctcaccagtccatatctttgaaaatcacaaagcagcagttctttgtgtgcagtggtgtccagacaggtcctctgtatttgggagtactgcagaggatggtcgtttgaatatttgggat

SEQ ID NO: 314 - DNA, 120 bp, ZEA MAYS
ggttctccaattcacaaatttgaaggccataaagctgctgttctttgtgttcagtggtcacctgacagagcatctgtttttggaagttctgcagaagatggtttcttaaacgtgtgggat

SEQ ID NO: 315 - DNA, 120 bp, ZEA MAYS
ggttctccaattcacaaatttgagggccataaagctgctgttctttgtgttcagtggtcacctgacagagcatctgtttttggaagttctgcagaagatggtttcttaaatgtttgggac

SEQ ID NO: 316 - DNA, 120 bp, SORGHUM BICOLOR
agttctccaattcataaatttgagggccataaagctgctgttctttgtgttcagtggtcacctgacagagcatctgtttttggaagttctgcggaagatggtttcttaaacgtgtgggat

SEQ ID NO: 317 - DNA, 120 bp, ORYZA SATIVA
ggcattccagttcacaaatttgagggtcataaagctgctgttctttgtgttcagtggtcacctgacaaggcatctgtatttggaagctctgcggaagacggcttcttaaatgtgtgggat

SEQ ID NO: 318 - DNA, 66 bp, ORYZA SATIVA
tggtcacctgacaaggcatctgtatttggaagctctgcggaagacggtttcttaaatgtgtgggat

SEQ ID NO: 319 - DNA, 120 bp, BRASSICA NAPUS
ggtacgcctatttacaaatttgaaggccacaaagctgctgttctttgcgttcagtggtctcctgataagtcatctgtttttgggagttccgcggaagatggtctcttgaacatctgggat

SEQ ID NO: 320 - DNA, 120 bp, PANICUM VIRGATUM
ggttctccaattcacaaatttgagggccataaagctgctgttctttgtgtccagtggtcacctgacagagcatctgttttcggaagttctgcggaagatggtttcttaaatgtgtgggat

SEQ ID NO: 321 - protein, 48 aa, MEDICAGO TRUNCATULA
SPGLFFRHAGHRDKVVDFHWNASDPWTIVSVSDDCASTGGGGTLQIWR

SEQ ID NO: 322 - protein, 48 aa, VITIS VINIFERA
PPGLFFRHAGHRDKVVDFHWNASDPWTIVSVSDDGESTGGGGTLQIWR

SEQ ID NO: 323 - protein, 48 aa, GOSSYPIUM HIRSUTUM
SAGLFFQHAGHRDKVVDFHWNAFDPWTVVSVFDDCETTGGGGTLQIWG

SEQ ID NO: 324 - protein, 48 aa, POPULUS
PAGLFFQHAGHRDKVVDFHWNASDPWTVVSVSDDCDTTGGGGTLQIWR

SEQ ID NO: 325 - protein, 48 aa, ARABIDOPSIS THALIANA
PAGLFFQHAGHRDKVVDFHWNASDPWTIVSVSDDCETTGGGGTLQIWR

SEQ ID NO: 326 - protein, 48 aa, GLYCINE MAX
PPGLFFQHAGHRDKVVDFHWNAYDPWTIVSVSDDCESTGGGGTLQIWR

SEQ ID NO: 327 - protein, 48 aa, PISUM SATIVUM; identical to SEQ ID NO: 326

SEQ ID NO: 328 - protein, 48 aa, MEDICAGO TRUNCATULA
PPGLFFQHAGHRDKVVDFHWNAHDPWTLVSVSDDCESTGGGGTLQIWR

SEQ ID NO: 329 - protein, 48 aa, PETUNIA HYBRIDA
APGLFFQHAGHRDKIVDFHWNVADPWTIVSVSDDCDSTGGGGTLQIWR

SEQ ID NO: 330 - protein, 48 aa, ZEA MAYS
PAGLFFQHAGHRDKIVDFHWNSSDPWTIVSVSDDGESTGGGGTLQIWR

SEQ ID NO: 331 - protein, 48 aa, ZEA MAYS; identical to SEQ ID NO: 330

SEQ ID NO: 332 - protein, 48 aa, SORGHUM BICOLOR; identical to SEQ ID NO: 330

SEQ ID NO: 333 - protein, 48 aa, ORYZA SATIVA; identical to SEQ ID NO: 330

SEQ ID NO: 334 - protein, 48 aa, ORYZA SATIVA; identical to SEQ ID NO: 330

SEQ ID NO: 335 - protein, 48 aa, BRASSICA NAPUS
PAGLFFQHAGHRDKVVDFHWNAEDPWTIVSVSDDCETTGGGGTLQIWR

SEQ ID NO: 336 - protein, 48 aa, PANICUM VIRGATUM; identical to SEQ ID NO: 330

SEQ ID NO: 337 - DNA, 144 bp, MEDICAGO TRUNCATULA
tcccctggtttattctttcgtcatgcagggcatagggataaggttgttgactttcattggaatgcatctgatccatggacaattgttagtgtatctgatgattgtgcaagcactggtggaggtggcaccctgcagatctggcgg

SEQ ID NO: 338 - DNA, 144 bp, VITIS VINIFERA
cctccaggtttattcttcagacatgctggccatagggataaggtcgtggacttccattggaatgcatcggatccatggacaatcgttagcgtatctgatgatggtgaaagtactggtggaggcggtacactccagatatggcgg

SEQ ID NO: 339 - DNA, 144 bp, GOSSYPIUM HIRSUTUM
tctgcgggactgtttttccagcacgctggacacagggacaaagttgttgacttccattggaatgcatttgatccatggactgttgttagtgtgtttgatgactgtgaaacaactggtggaggagggacattgcaaatatggggc

SEQ ID NO: 340 - DNA, 144 bp, POPULUS
cctgcaggattgtttttccagcatgctgggcacagggataaagttgttgatttccattggaatgcatctgatccttggacggtggttagtgtctctgatgactgtgataccactggcgggggagggacactgcagatatggcgc

SEQ ID NO: 341 - DNA, 144 bp, ARABIDOPSIS THALIANA
cccgctgggctcttcttccagcatgctggtcacagggacaaagttgttgatttccactggaatgcttcagacccttggactattgtcagtgtttctgatgactgtgagactactggtggaggtggaacattgcagatatggcgg

SEQ ID NO: 342 - DNA, 144 bp, GLYCINE MAX
cctccagggttgttttttcaacatgcaggtcatagggataaagttgttgacttccattggaatgcatatgatccatggacgattgttagtgtgtctgatgactgtgaaagtactggaggagggggaacgttgcagatatggcgc

SEQ ID NO: 343 - DNA, 144 bp, PISUM SATIVUM
cctccagggttgtttttccaacatgctggtcatagagacaaagttgttgacttccattggaatgcttatgatccatggacaattgtaagtgtgtctgatgattgtgaaagtactggtggaggaggaacattgcagatatggcgc

SEQ ID NO: 344 - DNA, 144 bp, MEDICAGO TRUNCATULA
cctccagggttgtttttccaacatgctggtcatagagacaaagttgttgactttcactggaatgcacatgatccatggacacttgttagtgtgtctgatgattgcgaaagtactggtggagggggaacattgcagatatggcgc

SEQ ID NO: 345 - DNA, 144 bp, PETUNIA HYBRIDA
gctccaggtttatttttccagcatgctgggcacagggataaaattgttgacttccactggaatgtggctgatccatggacaattgtaagtgtatctgatgactgtgactccacaggtggaggtggtacactacagatatggcgg

SEQ ID NO: 346 - DNA, 144 bp, ZEA MAYS
ccagccgggcttttctttcagcacgctggtcatagggataagattgtagacttccactggaattcgtcagatccttggacaattgtcagtgtctctgatgatggcgagagcactggtggaggtggaacactgcagatttggcgc

SEQ ID NO: 347 - DNA, 144 bp, ZEA MAYS
ccagctgggcttttctttcagcatgctggtcatagggataagatcgtagacttccactggaattcgtcagatccttggacaattgtcagtgtctcagatgatggtgagagcactggtggaggtggaacactgcagatatggcga

SEQ ID NO: 348 - DNA, 144 bp, SORGHUM BICOLOR
ccagctgggcttttcttccagcatgctggtcatagggataagattgtagacttccactggaattcgtcagatccttggacaattgtcagtgtatctgatgatggtgagagcactggtggaggcggaacactgcagatatggcgc

SEQ ID NO: 349 - DNA, 144 bp, ORYZA SATIVA
cctgctgggcttttctttcaacatgctggtcatagggataagattgtagacttccactggaattcttcggatccttggactattgtgagtgtgtctgatgatggtgagagtactggtggaggtggaacattgcagatatggcgc

SEQ ID NO: 350 - DNA, 144 bp, ORYZA SATIVA; identical to SEQ ID NO: 349

SEQ ID NO: 351 - DNA, 144 bp, BRASSICA NAPUS
ccggctggtctcttcttccagcatgctggtcacagggacaaagttgttgatttccactggaatgcagaggacccttggactattgtcagtgtttctgatgactgcgagactactggtggaggtggaacactgcagatatggcgg

SEQ ID NO: 352 - DNA, 144 bp, PANICUM VIRGATUM
ccagctgggcttttctttcaacatgctggtcacagggataagattgtagacttccactggaattcatcggatccttggacaatcgtcagtgtctcagatgatggtgagagcaccggtggaggtggaacactgcagatatggcgg

SEQ ID NO: 353-362 - skipped (10 entries)

SEQ ID NO: 363 - DNA, 227 bp, ARABIDOPSIS THALIANA
tgtttcctttgatatccgcactttaagcatgagagagtccctttgatattggcctggttcactcagatcttacctgaccacacacgtagatatacattattctctctagattatctgattgagccgcgccaatatctcagtactctctcgtctctattttggactttgtggtcttgtagatcgatttgtatgtgtgtgttgaaatggagacaagtacttgtaacttc

SEQ ID NO: 364 - DNA, 21 bp, ARTIFICIAL SEQUENCE
  Synthetic Primer
tgtttcctttgatatccgcac

SEQ ID NO: 365 - DNA, 22 bp, ARTIFICIAL SEQUENCE
  Synthetic Primer
gaagttacaagtacttgtctcc

SEQ ID NO: 366 - DNA, 66 bp, ARTIFICIAL SEQUENCE
  Synthetic Primer
cgggatccatgagagagtccctttcacgttctcgatttgtgcagagatcttacctgaccacacacg

SEQ ID NO: 367 - DNA, 65 bp, ARTIFICIAL SEQUENCE
  Synthetic Primer
gcgagctcacgagagagtactgacacgttctccatttgtgaagagataatctagagagaataatg

SEQ ID NO: 368 - DNA, 135 bp, ARTIFICIAL SEQUENCE
  Amplification fragment
ggatccatgagagagtccctttcacgttctcgatttgtgcagagatcttacctgaccacacacgtagatatacattattctctctagattatctcttcacaaatggagaacgtgtcagtactctctcgtgagctc

SEQ ID NO: 369 - DNA, 66 bp, ARTIFICIAL SEQUENCE
  Synthetic Primer
cgggatccatgagagagtccctttacaagtcagatgttttggggagatcttacctgaccacacacg

SEQ ID NO: 370 - DNA, 65 bp, ARTIFICIAL SEQUENCE
  Synthetic Primer
gcgagctcacgagagagtactgaacaagtcagttgttttgtggagataatctagagagaataatg

SEQ ID NO: 371 - DNA, 135 bp, ARTIFICIAL SEQUENCE
  Amplification fragment
ggatccatgagagagtccctttacaagtcagatgttttggggagatcttacctgaccacacacgtagatatacattattctctctagattatctccacaaaacaactgacttgttcagtactctctcgtgagctc

SEQ ID NO: 372 - DNA, 241 bp, ARTIFICIAL SEQUENCE
  Arabidopsis RNAi
tctagagatggaagtgtgcccaatactttggtcatagcaaattgtgaagttgttaagccaagggttgctgcagcagagcacatttctcagttcaatgaagaagcacgttctccaggccatctaggcctggagaacgtgcttcttcattgaactgagaaatgtgctctgctgcagcaacccttggcttaacaacttcacaatttgctatgaccaaagtattgggcacacttccatcgagctc

SEQ ID NO: 373 - DNA, 223 bp, ARTIFICIAL SEQUENCE
  Arabidopsis RNAi
tctagaatccaagatcacatcacaacgattgggacagattccaaatcatctggatctatcatcaaacagactggtgaaggtactgataagaatgagagtcctactggccatctaggccagtaggactctcattcttatcagtaccttcaccagtctgtttgatgatagatccagatgatttggaatctgtcccaatcgttgtgatgtgatcttggatgagctc

SEQ ID NO: 374 - DNA, 229 bp, ARTIFICIAL SEQUENCE
  Canola RNAi
tctagaatccaggaccacatcacaaccgctggtagtacagactccaaatcatctggatccatcatcaaacagactggtgaaggtggtgataagactgagagtccttctggccatctaggccagaaggactctcagtcttatcaccaccttcaccagtctgtttgatgatggatccagatgatttggagtctgtactaccagcggttgtgatgtggtcctggatgagctc

SEQ ID NO: 375 - DNA, 265 bp, ARTIFICIAL SEQUENCE
  Arabidopsis RNAi
tctagatcccgtccagatttgatactaactgggcaccaagataatgctgaatttgctcttgccatgtgcccaacggaaccctttgtgctctccggaggcaaggacaagtcagttgttttgtggagtggccatctaggccactccacaaaacaactgacttgtccttgcctccggagagcacaaagggttccgttgggcacatggcaagagcaaattcagcattatcttggtgcccagttagtatcaaatctggacgggagagctc

SEQ ID NO: 376 - DNA, 265 bp, ARTIFICIAL SEQUENCE
  Canola RNAi
tctagatcccgtcctgatttggtattaactggacatcaagacaatgctgaattcgctcttgcaatgtgcccaaccgaaccctttgtcctctctggaggcaaagacaagtcagttgttttgtggagtggccatctaggccactccacaaaacaactgacttgtctttgcctccagagaggacaaagggttcggttgggcacattgcaagagcgaattcagcattgtcttgatgtccagttaataccaaatcaggacgggagagctc

SEQ ID NO: 377 - DNA, 265 bp, ARTIFICIAL SEQUENCE
  Arabidopsis RNAi
tctagatttgtgaagaagtacaagaccatcattcaccctggagaggttaacagaatcagggaactcccacagaatagtaagattgttgctactcacaccgacagtcctgatgttctcatttgggatggccatctaggccatcccaaatgagaacatcaggactgtcggtgtgagtagcaacaatcttactattctgtgggagttccctgattctgttaacctctccagggtgaatgatggtcttgtacttcttcacaaagagctc

SEQ ID NO: 378 - DNA, 265 bp, ARTIFICIAL SEQUENCE
  Canola RNAi
tctagatttgtgaagaagttcaagaccatcattcaccctggagaggttaaccgaatcagggaactcccacaaaacagtaagattattgctactcacaccgacagtcctgatgttctcatttgggatggccatctaggccatcccaaatgagaacatcaggactgtcggtgtgagtagcaataatcttactgttttgtgggagttccctgattcggttaacctctccagggtgaatgatggtcttgaacttcttcacaaagagctc

SEQ ID NO: 379 - DNA, 265 bp, ARTIFICIAL SEQUENCE
  Arabidopsis RNAi
tctagagttggcccacgaggtgtatatcatggccatgaagatacagttgaagatgtggcattcagcccgacgagtgcacaagaattctgcagtgttggtgatgattcttgccttatactatgggatggccatctaggccatcccatagtataaggcaagaatcatcaccaacactgcagaattcttgtgcactcgtcgggctgaatgccacatcttcaactgtatcttcatggccatgatatacacctcgtgggccaacgagctc

(19) United States
(12) Patent Application Publication
(10) Pub. No.: US 2009/0094716 A1
(43) Pub. Date: Apr. 9, 2009

(54) PLANTS HAVING INCREASED BIOMASS

(75) Inventors: Jiangxin Wan, Bath (CA); Yafan Huang, Bath (CA); Zhiyong Zhang, Kingston (CA)

Correspondence Address: MINTZ, LEVIN, COHN, FERRIS, GLOVSKY AND POPEO, P.C, ONE FINANCIAL CENTER, BOSTON, MA, 02111, US

(73) Assignee: PERFORMANCE PLANTS, INC., Kingston (CA)

(21) Appl. No.: 12/237,282

(22) Filed: Sep. 24, 2008

Related U.S. Application Data
(60) Provisional application No. 60/974,623, filed on Sep. 24, 2007.

(51) Int. Cl. A01H 1/06; A01H 5/00; C07H 21/04

(52) U.S. Cl. 800/290; 800/298; 536/23.6

(57) ABSTRACT

The invention relates to methods of producing a desired phenotype in a plant by manipulation of gene expression within the plant. The method relates to means which inhibit the level of FVE gene expression or activity, wherein a desired phenotype, such as increased biomass relative to a wild-type control plant, is achieved. The invention also relates to nucleic acid sequences useful for such methods.

### REFERENCE TO RELATED APPLICATIONS

This application claims priority to U.S. Ser. No. 60/974,623, filed Sep. 24, 2007, which is herein incorporated by reference in its entirety.

### FIELD OF THE INVENTION

The invention relates generally to the field of plant molecular biology and production of plants having an improved growth characteristic. More specifically, the invention relates to FVE polynucleotides and polypeptides, including portions of an FVE polynucleotide, useful in producing a plant having an improved agronomic trait. Further, the invention relates to a plant having an improved agronomic trait relative to untransformed plants. A plant produced according to the invention has decreased FVE expression or activity produced by a transgenic or non-transgenic method.

### BACKGROUND OF THE INVENTION

Agriculture has always sought means of increasing the yield obtained from a given unit of land. As arable land is limited there has always been development of crops having desirable characteristics. Conventionally, this has been through identification of valuable plants, breeding for desirable traits and production of hybrid plants. Genetic engineering has introduced a variety of methods to target genetic improvements to plants including the introduction of novel genes or gene constructs into plants that produce improved traits. In this way new cellular activities can be introduced or an existing gene activity may be modified. Additionally, molecular biology has provided means of introducing greater genetic variability into a population and selecting the plants that have desirable traits. More recently methods of introducing specific genetic manipulations into a plant have been developed. All of the methods have the desired result of producing and or identifying a plant having desirable traits, be that yield, environmental stress tolerance or novel product production for example. Yield is a variable term depending on the trait of interest. In some instances yield refers to production of seed or a component of a seed such as starch, oil or protein. Alternatively, yield may refer to production of total biomass or of specific plant parts, for example, tubers, leaf biomass, stem or plant architecture.

Improvement in biomass production has been a historical target for trait improvement. For example, in farm practice that has included production of improved forage crops or in silviculture production of trunk or branch material. A variety of plant material has been found useful in the production of alternative energy sources, be it as a petroleum additive or replacement such as ethanol or biodiesel, incineration technologies, gasification and pyrolysis processes. Means of increasing the biomass production of a plant will be beneficial to a variety of applications.

Herein, an FVE gene has been identified as a loss of function mutant that modifies the plant growth and development and finds utility in a method of increasing plant biomass and total plant production. The FVE gene encodes a protein having 6 copies of a WD40 domain motif. In Arabidopsis, WD40 motifs have been found in at least 237 proteins, in 143 distinct gene families (Nocker and Ludwig, 2003) and are believed to be involved generally as a component of transcription regulators controlling a variety of cellular processes.

AtFVE has also been reported as AtMSI4, a plant homolog of yeast MSI (multicopy suppressor of IRAI) and mammalian retinoblastoma-associated proteins RbAp46 and RbAp48 (Kenzior and Folk, 1998). A small gene family of 5 MSI-like genes was found in Arabidopsis genome (Ach et al., 1997). AtMSI5 is the only MSI member sharing high homology (75% identity nucleic acid) to AtFVE (AtMSI4), suggesting they may be functionally related. Although AtMSI1 has been reported to be involved in regulation of flowering time, it doesn't share the same target gene as AtFVE (Hennig et al., 2003). Within the literature FVE has also been referred to as ACG1, NFC4 and NFC04.

The FVE gene has been found to be a component of the autonomous pathway of flowering time regulation. In studies to date FVE has been regarded as a repressor of FLC, which plays a central role in regulation of flowering time. Regulation of FLC is influenced by numerous pathways and the more numerous genes that make up these pathways, of which FVE is but one of these. The Arabidopsis FLC was expressed in tobacco and shown to delay flowering and increase biomass. However, as there are numerous inputs into FLC regulation it is not apparent what upstream component would permit the generation of desirable phenotypes. Furthermore a FLC orthologue has only been identified in Brassica and there is no known FLC orthologue identified outside of the Brassicaceae family, of which Arabidopsis is a member.

Genetic regulation of flowering time in response to environmental cues and developmental signals has been studied in Arabidopsis. Detection of these cues involves the light-dependent pathway (FRI, FRL1, FL2, and FES1); the vernalization pathway which initiates early flowering by cold treatment (VIN3, VRN1, VRN2, and LHP1); the autonomous pathway which mediates developmental signals (FCA, FY, FLD, FPA, FVE, LD, and FLK); and the gibberellin pathway promoting flowering by action of the hormone. The light-dependent pathway promotes the floral activator CONSTANS(CO), and in turn, activates the floral pathway integrators FLOWERING TIME (FT), SUPPRESSOR OF OVEREXPRESSION OF CONSTANS1 (SOC1), and LEAFY (FLY) that are also positively regulated by the gibberellin pathways. In a parallel manner, the vernalization and autonomous pathways repress FLOWERING LOCUS C (FLC), a suppressor of the floral pathway integrators.

An Arabidopsis fve mutant is described by Ausin et al., Nature Genetics, Vol:36(2), 162-166. The mutant is described as having a late flowering phenotype. The wild-type FVE gene was over-expressed in a wild-type Columbia background with little observable phenotype to only a slight reduction in time to flowering. The authors conclude that FVE is not sufficient to significantly alter flowering time and that FVE polypeptide concentration in the cell seems not to be a limiting factor and FVE function may require other protein factors.

This invention is directed at the manipulation of FVE polynucleotides and polypeptides to decrease FVE expression or activity in a plant cell, tissue culture or plant. Inhibition of the FVE expression or activity provides a plant having an improved agronomic property, for example, increased biomass yield relative to a wild-type control plant.

### SUMMARY OF THE INVENTION

The present invention provides a method of producing a plant having an improved property, wherein the method includes inhibiting the expression or activity of an endogenous gene, wherein a plant is produced having an advantageous phenotype or improved property. In a particular embodiment, the present invention provides a method for producing plants having increased biomass, wherein the method includes include generation of transgenic plants and modification of plants genome using the methods described herein.

Biomass refers to an amount of plant material either in terms of dry weight or fresh weight as appropriate, and includes all plant parts, such as in reference to shoot biomass (all above ground plant parts), leaf biomass, and root biomass. As used herein, the term “increased biomass” refers to a plant biomass that is 2, 4, 5, 6, 8, 10, 20 or more fold greater as compared to the biomass of a corresponding wild-type plant. For example, a plant having increased biomass as compared to a wild-type plant may have 10%, 15%, 20%, 25%, 30%, 40%, 50%, 60% 70%, 75% or greater biomass than the corresponding wild-type plant.

The methods of the invention involve inhibiting the expression or activity of an endogenous gene, such as FVE, wherein a plant is produced having an advantageous phenotype or improved property, such as increased biomass. In one embodiment, the invention provides a method of producing a plant having increased biomass accumulation relative to a wild-type plant, by introducing into a plant cell a nucleic acid construct that inhibits the expression or activity of FVE. For example, a plant having increased biomass accumulation relative to a wild type plant is produced by a) providing a nucleic acid construct containing a promoter operably linked to a nucleic acid construct that inhibits FVE activity; b) inserting the nucleic construct into a vector; c) transforming a plant, tissue culture, or a plant cell with the vector to obtain a plant, tissue culture or a plant cell with decreased FVE activity; d) growing the plant or regenerating a plant from the tissue culture or plant cell, wherein a plant having increased biomass accumulation relative to a wild type plant is produced.

The term “nucleic acid construct” refers to a full length gene sequence or portion thereof, wherein a portion is preferably at least 19, 20, 21, 22, 23, 24, 25, 30, 40, 50, 60, 70, 75, 80, 90, 100, or 150 nucleotides in length, or the compliment thereof. Alternatively it may be an oligonucleotide, single or double stranded and made up of DNA or RNA or a DNA-RNA duplex. In a particular embodiment, the nucleic acid construct contains the full length FVE gene sequence, or a portion thereof, wherein the portion of the FVE sequence is at least 19, 20, 21, 22, 23, 24, 25, 30, 40, 50, 60, 70, 75, 80, 90, 100, or 150 nucleotides in length, or its compliment. In some embodiments, the portion of the FVE sequence includes an FVE-S1 motif, an FVE-S2 motif, or an FVE-WD40 motif.

Also provided by the invention is a transgenic plant having an advantageous phenotype or improved property such as increased biomass, produced by the methods described herein. In one embodiment, the invention provides a plant having a non-naturally occurring mutation in an FVE gene, wherein the plant has decreased FVE expression or activity and the plant has increased biomass relative to a wild-type control. Decreased FVE expression or activity refers to a 2, 3, 4, 5, 6, 7, 8, 9, 10, 15, 20, 25, 30, 40, 50, 60, or 75-fold reduction or greater, at the DNA, RNA or protein level of an FVE gene as compared to wild-type FVE, or a 2, 3, 4, 5, 6, 7, 8, 9, 10, 15, 20, 25, 30, 40, 50, 60 or 75 fold reduction of FVE activity as compared to wild-type FVE activity. FVE activity includes but is not limited to regulation of flowering time, and response (acclimation) to cold.

The invention further provides a transgenic seed produced by the transgenic plant(s) of the invention, wherein the seed produces plant having an advantageous phenotype or improved property such as for example, increased biomass accumulation relative to a wild-type plant.

In another embodiment, the invention provides nucleic acids for expression of nucleic acids in a plant cell to produce a transgenic plant having an advantageous phenotype or improved property such as increased biomass. Such nucleic acids include SEQ ID NO's:1, 5, 206, 216, 220, 239, 241, 368, 371, 372, 373, 374, 375, 376, 377, 378, and 379. The invention further provides compositions which contain the nucleic acids of the invention for expression in a plant cell to produce the transgenic plants described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

### BRIEF DESCRIPTION OF THE FIGURES

FIG. 1 is an alignment of the WD-40 domain structure of FVE in Arabidopsis and selected major crops.

### DETAILED DESCRIPTION

The invention is based in part on the discovery of plants having an improved agronomic property, such as for example, increased biomass yield relative to a wild-type control. More specifically, the invention is based upon the discovery of a mutant FVE gene having reduced activity. Plants having this mutant FVE gene were observed to have increased biomass as compared to plants not having the mutant FVE gene.

Accordingly the invention provides methods of enhancing (e.g., increasing) the biomass of plants by decreasing the expression or activity of a. FVE gene or polypeptide. FVE expression or activity is decreased by methods know in the art. For example, a plant having increased biomass as compared to a wild-type (e.g. control) plant is produced by introducing to a plant cell a nucleic acid construct that decreases the expression or activity of an FVE gene or polypeptide. The invention also includes the transgenic plants produced by the methods of the invention and the seeds produced by the transgenic plants that produce a plant having increased biomass.

For convenience, before further description of the present invention, certain terms employed in the specification, examples and appended claims are defined herein. These definitions should be read in light of the remainder of the disclosure and as understood by a person of ordinary skill in the art.

A “promoter sequence”, or “promoter”, means a nucleic acid sequence capable of inducing transcription of an operably linked gene sequence in a plant cell The term “expression cassette” means a construct, or portion thereof, comprising a gene sequence under the regulatory control of a 5′ sequence, and optionally a 3′ transcription termination sequence, wherein the gene sequence is transcribed. Additionally, the expressed mRNA may be translated into a polypeptide.

The terms “expression” or “over-expression” are used interchangeably and mean the expression of a gene such that the transgene is expressed. The total level of expression in a cell may be elevated relative to a wild-type cell.

The term “FVE nucleic acid” refers to at least a portion of a nucleic acid which encodes an FVE gene sequence. Similarly the term “FVE protein” or “FVE polypeptide” refers to at least a portion thereof. A portion of an FVE nucleic acid is at least 19, 20, 21, 22, 23, 24, 25, 30, 40, 50, 60, 70, 75, 80, 90, 100, or 150 nucleotides in length with respect to a nucleic acid which encodes an FVE gene, and a portion of a protein or polypeptide is at least 7, 8, 9, 10, 11, 12, 13, 14, 15, 20, 25, 30, 35, 40, 45 or 50 amino acids in length with respect to the amino acid sequence of an FVE gene.

The term “nucleic acid construct” means a full length gene sequence or portion thereof, wherein a portion is preferably at least 19, 20, 21, 22, 23, 24, 25, 30, 40, 50, 60, 70, 75, 80, 90, 100, or 150 nucleotides in length, or the compliment thereof. Alternatively it may be an oligonucleotide, single or double stranded and comprised of DNA or RNA or a DNA-RNA duplex. Optionally, a “nucleic acid construct” may further include an operably linked promoter and an operably linked terminator region so as to form an expression cassette.

The term “biomass” refers to an amount of plant material either in terms of dry weight or fresh weight as appropriate. Biomass includes all plant parts unless otherwise stipulated, such as in reference to shoot biomass (all above ground plant parts), leaf biomass, and root biomass.

The term “increased biomass” refers to a plant biomass that is 2, 4, 5, 6, 8, 10, 20 or more fold greater as compared to the biomass of a corresponding wild-type plant. For example, a plant having increased biomass as compared to a wild-type plant may have 10%, 15%, 20%, 25%, 30%, 40%, 50%, 60% 70%, 75% or greater biomass than the corresponding wild-type plant.

The term “wild-type” refers to a plant or plant cell of the same species, wherein FVE expression has not been altered, mutated, or modulated by any natural or non-natural means.

The term “dry weight” means plant tissue that has been dried to remove the majority of the cellular water and is used synonymously and interchangeably with the term biomass.

The terms “decrease FVE expression or activity”, “decreased FVE expression or activity”, “inhibit FVE expression or activity”, and “inhibition of FVE expression or activity” are used herein interchangeably, and refer to a 2, 3, 4, 5, 6, 7, 8, 9, 10, 15, 20, 25, 30, 40, 50, 60, or 75-fold reduction or greater, at the DNA, RNA or protein level of an FVE gene as compared to wild-type FVE, or a 2, 3, 4, 5, 6, 7, 8, 9, 10, 15, 20, 25, 30, 40, 50, 60 or 75 fold reduction of FVE activity as compared to wild-type FVE activity. FVE activity includes but is not limited to regulation of flowering time, and response (acclimation) to cold.

For clarity the use of gene names have followed the following format. Mutant genes (nucleic acids) in lower case italics; wild-type genes (nucleic acids) in upper case italics; proteins in regular font and lower case for mutant and regular case and upper case for wild-type. In some cases the contextual meaning may prevail over a format designation.

### Sequences

The following sequences and corresponding sequence identifiers are employed throughout the specification, examples and appended claims:

| | | |
|---|---|---|
| SEQ ID NO: 1 | Atfve b10 | nucleotide |
| SEQ ID NO: 2 | Atfve b10 | polypeptide |
| SEQ ID NO: 3 | AtFVE WT | nucleotide |
| SEQ ID NO: 4 | AtFVEWT | polypeptide |
| SEQ ID NO: 5 | Atfve b10 | Genomic |
| SEQ ID NO: 6 | AtFVE WT | Genomic |
| SEQ ID NO: 7 | FVE cotton | nucleotide |
| SEQ ID NO: 8 | FVE cotton | polypeptide |
| SEQ ID NO: 9 | FVE soybean | nucleotide |
| SEQ ID NO: 10 | FVE soybean | polypeptide |
| SEQ ID NO: 11 | FVE petunia | nucleotide |
| SEQ ID NO: 12 | FVE petunia | polypeptide |
| SEQ ID NO: 13 | FVE P. sativum | nucleotide |
| SEQ ID NO: 14 | FVE P. sativum | polypeptide |
| SEQ ID NO: 15 | FVE poplar | nucleotide |
| SEQ ID NO: 16 | FVE poplar | polypeptide |
| SEQ ID NO: 17 | FVE grape | nucleotide |
| SEQ ID NO: 18 | FVE grape | polypeptide |
| SEQ ID NO: 19 | FVE sorghum | nucleotide |
| SEQ ID NO: 20 | FVE sorgham | polypeptide |
| SEQ ID NO: 21 | FVE maize-1 | nucleotide |
| SEQ ID NO: 22 | FVE maize-1 | polypeptide |
| SEQ ID NO: 23 | FVE maize-2 | nucleotide |
| SEQ ID NO: 24 | FVE maize-2 | polypeptide |
| SEQ ID NO: 25 | FVE rice-1 | nucleotide |
| SEQ ID NO: 26 | FVE rice-1 | polypeptide |
| SEQ ID NO: 27 | FVE rice-2 | nucleotide |
| SEQ ID NO: 28 | FVE rice-2 | polypeptide |
| SEQ ID NO: 29 | FVE medicago-1 | nucleotide |
| SEQ ID NO: 30 | FVE medicago-1 | polypeptide |
| SEQ ID NO: 31 | FVE medicago-2 | nucleotide |
| SEQ ID NO: 32 | FVE medicago-2 | polypeptide |
| SEQ ID NO: 33 | FVE silene latifoloa | nucleotide |
| SEQ ID NO: 34 | FVE silene latifoloa | polypeptide |
| SEQ ID NO: 35 | FVE physcomitrella | nucleotide |
| SEQ ID NO: 36 | FVE physcomitrella | polypeptide |
| SEQ ID NO: 206 | FVE Brassica | nucleotide |
| SEQ ID NO: 207 | FVE Brassica | polypeptide |
| SEQ ID NO: 216 | FVE Switchgrass | nucleotide |
| SEQ ID NO: 217 | FVE switchgrass | polypeptide |
| SEQ ID NO: 220 | FVE Maize-3 | nucleotide |
| SEQ ID NO: 221 | FVE maize-3 | polypeptide |
| SEQ ID NO: 239 | FVE soybean-2 | nucleotide |
| SEQ ID NO: 240 | FVE soybean-2 | polypeptide |
| SEQ ID NO: 241 | FVE Brachypodium | nucleotide |
| SEQ ID NO: 242 | FVE Brachypodium | polypeptide |
| SEQ ID NO: 363 | At miRNA171 pre | nucleotide |
| SEQ ID NO: 368 | At amiR171-FVE-S1 | nucleotide |
| SEQ ID NO: 371 | At amiR171-FVE-WD2 | nucleotide |
| SEQ ID NO: 372-379 | Hairpin-RNAi | nucleotide |
| | expression cassettes | |
| SEQ ID NO: 37-50, 243, 244 | S1 motif | polypeptide |
| SEQ ID NO: 51-64, 245, 246 | S1 motif | nucleotide |
| SEQ ID NO: 65-78, 223, 247 | S2 motif | polypeptide |
| SEQ ID NO: 79-92, 233, 248 | S2 motif | nucleotide |
| SEQ ID NO: 93-106, 228, 251 | WD40-2 motif | polypeptide |
| SEQ ID NO: 107-120, 234, 252 | WD40-2 motif | nucleotide |
| SEQ ID NO: 121-134, 236, 249 | WD40-1 motif | polypeptide |
| SEQ ID NO: 135-148, 235, 250 | WD40-1 motif | nucleotide |
| SEQ ID NO: 149-162, 255, 253 | WD40-3 motif | polypeptide |
| SEQ ID NO: 163-176, 256, 254 | WD40-3 motif | nucleotide |
| SEQ ID NO: 257-272 | WD40-4 motif | polypeptide |
| SEQ ID NO: 273-288 | WD40-4 motif | nucleotide |
| SEQ ID NO: 289-304 | WD40-5 motif | polypeptide |
| SEQ ID NO: 305-320 | WD40-5 motif | nucleotide |
| SEQ ID NO: 321-336 | WD40-6 motif | polypeptide |
| SEQ ID NO: 337-352 | WD40-6 motif | nucleotide |

### Determining Homology Between Two or More Sequences

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the coding sequence portion of the DNA sequence shown in SEQ ID NO: 1.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

### AtFVE Antibody Preparation:

Polyclonal antibodies to FVE were produced using purified wild-type Arabidopsis FVE protein as an antigen. A full length cDNA of wild-type AtFVE was PCR amplified using the primer pair SEQ ID NO's: 199 and 200 and cloned into the expression vector pMAL-c2h using BamH I and Sal I restriction sites.

The bacteria strain DH5a was used as a host to express AtFVE. A positive clone that produced fusion proteins of maltose binding protein (MBP) and AtFVE of the correct size (99 KD) were selected for protein expression in large scale. After affinity purification through amylase resin, the fusion protein was separated in SDS-PAGE gel, then recovered and eluted through an Electro-Eluter and then condensed with Centriprep YM-30 and Centricom YM-30. Total of 500 μg of MBP-AtFVE fusion protein were obtained and 400 μg of MBP-AtFVE protein were used for the antibody preparation.

For antibody production, purified protein was diluted with PBS and mixed with an adjuvant of Titermax Gold. Pre-immune sera were collected as control and kept at −80° C. For the first injection, 150 μg of MBP-AtFVE were injected into a rabbit; three weeks after the first injection, second injection of 150 μg fusion protein was performed. Two weeks after the second injection, a third injection of 100 μg was administered. The final anti-sera were collected 1 week after the third injection and the antibody titer was determined.

### Inhibition of Endogenous FVE Expression and Activity

An aspect of the invention pertains to means and methods of inhibiting FVE expression and activity. There are numerous methods known to those skilled in the art of achieving such inhibition that effect a variety of steps in a gene expression pathway, for example transcriptional regulation, post transcriptional and translational regulation. Such methods include, but are not limited to the use of antisense oligonucleotides; RNA interference (RNAi), including short interfering RNAi (siRNA) and short hairpin constructs (shRNA); microRNA (miRNA), including artificial miRNA (amiRNA) (Schwab et al., The Plant Cell 18:1121-1133 (2006)) technologies; aptamers; ribozymes; mutagenesis and TILLING methods; in vivo site specific mutagenesis techniques; and dominant/negative inhibition approaches.

One method of inhibiting FVE expression and activity involves the use of an FVE antisense nucleic acid. Antisense nucleic acids do not act catalytically to degrade mRNA, but instead involve single-stranded RNA fragments which physically bind to mRNA and block protein translation (see e.g., Weiss et al., Cell. Mol. Life. Sci., 55:334-358 (1999)). As used herein, an FVE “antisense” nucleic acid refers to a nucleic acid capable of hybridizing by virtue of some sequence complementary to a portion of an RNA (preferably mRNA) encoding FVE. The antisense nucleic acid may be complementary to a coding and/or noncoding region of an mRNA encoding FVE. Such antisense nucleic acids have utility as compounds that inhibit FVE expression, and can be used to produce a plant having an improved agronomic trait. The antisense nucleic acids of the invention are double-stranded or single-stranded oligonucleotides, RNA or DNA or a modification or derivative thereof, and can be directly administered to a cell or produced intracellularly by transcription of exogenous, introduced sequences. The antisense portion needs not be a full length gene nor be 100% identical. Provided that the antisense is at least about 70% identical to the endogenous target gene and of about 50 nucleotides or greater in length the desired inhibition will be obtained. The expressed gene may be heterologous to the transformed species, for example an Arabidopsis gene may be introduced into a heterologous species such as Brassica, soybean, maize, wheat, or grasses.

The antisense oligonucleotides can be DNA or RNA or chimeric mixtures or derivatives or modified versions thereof and can be single-stranded or double-stranded. In addition, the antisense molecules may be polymers that are nucleic acid mimics, such as PNA, morpholino oligos, and LNA (see e.g., Morcos, et al., Biochem Biophys Res Commun 358(2): 521-7 (2007).

Another method of FVE inhibition involves RNA interference (also referred to as “RNA-mediated interference” (RNAi) or post-transcriptional gene silencing (PTGS)) through the use of short interfering RNAs (siRNA) and/or or short hairpin RNA (shRNA) constructs. RNAi is a mechanism that inhibits gene expression at the stage of translation or by hindering the transcription of specific genes (see, e.g., Fire et al., Nature 391(6669):806-11 (1998)). siRNA are double-stranded RNA molecules that are involved in the RNAi pathway (see, e.g., Hamilton et al., Science 286(5441):950-2 (1999)). A portion of the gene to be inhibited (e.g., FVE) is used and cloned in a sense and antisense direction having a spacer separating the sense and antisense portions. The size of the gene portions should be at least 20 nucleotides in length and the spacer may be a little as 13 nucleotides in length (see e.g., Kennerdell and Carthew, 2000), and preferably should be at least 50 nucleotides in length and the spacer may be a little as 4 nucleotides. A spacer sequence may be an intron sequence, a coding or non-coding sequence, or formed from the ligation at a splice site such as SfiI. The sequence selected for an RNAi construct should have specificity to the native target gene and should not alter the expression of non-target genes. For example, if a WD40 motif, or portion thereof, is selected it should be distinct from WD40 motifs of non-target genes, i.e. non-FVE genes.

Specific RNAi pathway proteins are guided by the siRNA to the targeted messenger RNA (mRNA), where they “cleave” the target, breaking it down into smaller portions that can no longer be translated into protein. The RNAi pathway is initiated by the enzyme dicer, which cleaves long, dsRNA molecules into short fragments of 20-25 base pairs. One of the two strands of each fragment, known as the guide strand, is then incorporated into the RNA-induced silencing complex (RISC) and pairs with complementary sequences. SiRNAs can be exogenously (artificially) introduced into cells by various transfection methods known to those of skill in the art to bring about the specific knockdown of a gene of interest. shRNA is a sequence of RNA that makes a tight hairpin turn that can be used to silence gene expression via RNA interference. The shRNA hairpin structure is cleaved by the cellular machinery into siRNA, which is then bound to the RNA-induced silencing complex (RISC). This complex binds to and cleaves mRNAs which match the siRNA that is bound to it. Accordingly, siRNAs/shRNAs capable of targeting and cleaving homologous FVE mRNA are useful for producing a plant having an improved agronomic trait.

Alternatively, artificial microRNA (amiRNA) inhibition can be used to inhibit gene expression and activity in a more specific manner. In contrast to small inhibitory RNA (siRNA) that requires a perfect match between the siRNA and the target mRNA, amiRNA allows up to 5 mismatches with no more than 2 consecutive mismatches. The construction of amiRNA needs to meet certain criteria described in Schawab et al., 2006. This provides a method to down-regulate a target gene expression and/or activity using a gene portion comprising of at least a 21 nucleotide sequence of the target gene, such as FVE. Artificial microRNA approaches can be applied more broadly for down-regulation of genes within a plant gene family. A single construct can be used to down regulate the homologs in closely related species. For example, if an amiRNA construct is based on a conserved region between Arabidopsis and canola, the same construct can be used to regulate the endogenous gene in both Arabidopsis and canola. Another example, a single common amiRNA construct can be used to down regulate homologous targets in several cereal species such as brachypodium, miscanthus, switchgrass, maize, sorghum and rice.

The first micro RNA (miRNA) lin-4 was discovered in 1993 by Victor Ambros (Cell 75, 843-854, 1993). The miRNAs originate from capped & polyadenylated full length precursor-miRNA (pri-miRNA). Through a series of processing steps, a pri-miRNA produces a pre-miRNA then to a mature miRNA of about 21 nucleotides. miRNAs in plants were first identified and reported in Brenda J. Reinhart et al., Genes & Development 16:1616-1626, (2002). Endogenous miRNA sequences have been modified and used to produce artificial microRNA for gene silencing of desired target genes. For example, amiRNA171s have been reported to down-regulate target genes (Eneida Abreu Parizotto, et al., Genes & Development 18:2237-2242, (2004); Jing Qu et al., Journal of Virology, 81 (12): 6690-6699 (2007)).

Artificial miRNAi cassettes can be engineered using a PCR strategy that uses a first PCR reaction to amplify a precursor miRNA which serves as template for a second PCR reaction using primers that introduce the desired target gene sequence, and optionally a cloning site, into the product and amplifies the loop portion of the precursor amiRNA. This forms the amiRNA cassette that can then be cloned into a vector or transformation vector for introduction into a plant cell.

Another method of FVE inhibition involves the use of aptamers. Aptamers are capable of specifically binding to selected targets and modulating the target's activity, e.g., through binding aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). A series of structural studies have shown that aptamers are capable of using the same types of binding interactions (e.g., hydrogen bonding, electrostatic complementarities, hydrophobic contacts, steric exclusion) that drive affinity and specificity in antibody-antigen complexes. As such, aptamers have utility as compounds that inhibit FVE expression, and can be used to produce a plant having an improved agronomic trait (e.g., increased biomass). As used herein, an FVE aptamer refers to a nucleic acid capable of specifically binding and modulating (e.g., inhibiting) the function of FVE. The process known in the art as SELEX (Systematic Evolution of Ligands by Exponential Enrichment) can be used to generate FVE aptamers, as described in U.S. Pat. Nos. 5,475,096 and 5,270,163.

Yet another method of FVE inhibition involves the reduction of FVE expression and activity by using enzymatic nucleic acid molecules. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. Ribozymes, for example, can be designed to catalytically cleave gene mRNA transcripts encoding FVE, preventing translation of target gene mRNA and, therefore, expression of the gene product. The mechanism of ribozyme action involves sequence-specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage (see, e.g., U.S. Pat. No. 5,093,246). While ribozymes that cleave mRNA at site-specific recognition sequences can be used to destroy mRNAs encoding FVE, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of ribozymes (including modified ribozymes) is well known in the art (see e.g., U.S. Pat. No. 6,448,009; U.S. Pat. No. 6,890,908; Cammeron and Jennings (Proc. Natl. Acad. Sci. USA 86 (1986), 9139-9143); Cotten et al. (Mol. Cell. Biol. 9 (1989), 4479-4487; Perreault et al. (Nature 344 (1990), 565-567)).

The FVE antisense nucleic acids, siRNA/shRNA constructs, aptamers, and/or ribozymes of the present invention may be modified at a base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see e.g., Hyrup, B. and Nielsen, P. E. (1996) BIOORG. MED. CHEM. 4(1):5-23). Peptidyl nucleic acids have been shown to hybridize specifically to DNA and RNA under conditions of low ionic strength.

Dominant-negative inhibition is analogous to competitive inhibition of biochemical reactions. Expression of a modified or mutant polypeptide that lacks full functionality competes with the wild-type or endogenous polypeptide thereby reducing the total gene/protein activity. For example an expressed protein may bind to a protein complex or enzyme subunit to produce a non-functional complex or a complex having reduced levels of activity. Alternatively the expressed protein may bind substrate but not have full activity to perform the native function. Expression of sufficient levels of non active protein will reduce or inhibit the overall function. The strategies of dominant negative effects have been applied to study gene function by changing the activity of an endogenous gene through over-expression of inactive proteins, or over-expression of mutant transcription factors lacking DNA-binding domains, or over-expression of isolated DNA-binding domains. The over-expressed inactive protein will compete with the wild-type form of the protein resulting in dominant negative effects. The over-expression of a mutant transcription factor lacking a DNA-binding domain may form heterodimers with endogenous factors or may titrate interacting components of the transcription machinery, thereby preventing the endogenous factor from binding to DNA. An over-expressed isolated DNA-binding domain will occupy the endogenous DNA-binding sites such that the endogenous factor cannot bind to DNA and therefore can not make the necessary protein-protein contacts for transcription.

Expression of an FVE gene that produces an altered FVE protein that is lacks full functionality can be used for dominant-negative down-regulation of gene activity. An altered FVE polypeptide is produced that, for example, may associate with or bind to a target molecule but lacks endogenous activity. A target molecule may be an interacting protein or a nucleic acid sequence. In this manner the endogenous FVE protein is effectively diluted and downstream responses will be attenuated.

For example, expression of an fve-b10 (SEQ ID NO: 1) will produce a protein that has an altered amino acid sequence relative to a wild-type FVE gene and lacks full activity resulting in a plant having inhibited FVE expression and activity thereby producing a plant having an advantageous phenotype or improved property such as increased biomass. An altered FVE-like sequence such as Atfve-b10 may have an additional polypeptide sequence that alters the conformation of the protein folding and alters the activity of the protein. Other FVE coding sequences can be produced that will insert or delete amino acids that result in a change in protein folding or conformation. The resulting proteins that do not have full activity are useful for expression in a dominant-negative type down-regulation strategy.

In vivo site specific mutagenesis is now available whereby one can introduce a mutation into a cells genome to create a specific mutation. The method as essentially described in Dong et al. Plant Cell Reports 25:457-465 (2006), or U.S. patent application publication number 20060162024, which refer to the methods of oligonucleotide-directed gene repair. Alternatively one may use chimeric RNA/DNA oligonucleotides essentially as described Beetham, Proc. Natl. Acad. Sci. USA, Vol. 96 pp. 8774-8778 (1999), or in Okuzaki and Toriyama, Plant Cell Reports, 22:509-512 (2004). Accordingly, a premature stop codon may be generated in the cells' endogenous gene thereby producing a specific null mutant. Alternatively, the mutation may interfere with splicing of the initial transcript thereby creating a non-translatable mRNA or an mRNA that produces an altered polypeptide which does not possess endogenous activity. Changes may also be introduced into the native promoter region to reduce gene transcription and thereby reduce or eliminate gene expression and activity.

TILLING is a method of isolating mutations in a known gene from an EMS-mutagenized population. The population is screened by methods essentially as described in (Greene et al., 2003). Provided with the disclosure concerning which gene is the best target gene, an FVE gene in this case, the TILLING methods are routine to isolate a plant having the desired mutation that inhibits gene activity.

Zinc fingers can be used to up-regulate or down-regulate any gene in a plant as described by Choo et al., in US Application 2008/0070306 and related patents and patent applications. By designing a zinc finger with a transactivating domain the induction of an endogenous gene can be accomplished specifically and bypass any endogenous regulation of the targeted gene. Previously, the only available method was to introduce a transgene in another location of the genome under the regulation of a separate promoter. The zinc fingers of the present invention can also be used to down-regulate any gene in any plant, which has previously only been possible using techniques such as, but not limited to, antisense, ribozymes, co-suppression and RNAi methods including hair-pinRNA, siRNA, microRNA and artificial-microRNA. The zinc finger approach to down-regulation is highly potent and allows the targeting of specific member of a gene family without affecting the other members.

A zinc finger chimera is a transcription factor that comprises a DNA binding domain (comprising a number of zinc fingers), designed to bind specifically to any DNA sequence and one or more further domains. Usually, a nuclear localization domain is attached to the zinc finger domain to direct the chimera to the nucleus. Generally, the chimera also includes an effector domain that can be a transactivation or repression domain to regulate the expression of the gene in question (see e.g., Choo and Klug, Curr. Opin. Biotech. 6:431-436 (1995); Choo and Klug (1997); Rebar and Pabo, Science 263:671-673 (1994); and Jamieson et al. Biochem. 33:5689-5695 (1994)). The zinc finger chimera may also include other domains which may be advantageous. For example, DNA modifying domains (such as endonucleases and methylases) can be added to the zinc finger domain, conferring to the zinc finger chimera the ability to regulate expression of the gene of interest or modify any DNA specifically (see e.g., Wu et al., Proc. Natl. Acad. Sci. USA 92:344-348 (1995); Nahon and Raveh (1998); Smith et al. (1999); and Carroll et al. (1999)).

In a conceived embodiment, transcription of the FVE gene is modulated using zinc-finger derived transcription factors (ZFPs). These ZFPs comprise both a DNA recognition domain and a functional domain that causes activation or repression of a target nucleic acid such as an FVE nucleic acid. Therefore, activating and repressing ZFPs can be created that specifically recognize the FVE promoters described above and used to increase or decrease FVE expression in a plant, thereby modulating the phenotypes of the plant. Methods of altering a gene expression are described in US Application 2008/0070306 and related patents and patent applications.

Other strategies of gene inhibition will be apparent to the skilled worker including those not discussed here and those developed in the future.

### Identification of FVE Homologues

Arabidopsis FVE homologs were identified using database sequence search tools, such as the Basic Local Alignment Search Tool (BLAST) (Altschul et al. 1215:403-410 (1990); and Altschul et al. Nucl. Acids Res. 25: 3389-3402 (1997)). The tblastn or blastn sequence analysis programs were employed using the BLOSUM-62 scoring matrix (Henikoff, S, and Henikoff, J. G. Proc. Natl. Acad. Sci. USA 89:10915-10919). The output of a BLAST report provides a score that takes into account the alignment of similar or identical residues and any gaps needed in order to align the sequences. The scoring matrix assigns a score for aligning any possible pair of sequences. The P values reflect how many times one expects to see a score occur by chance. Higher scores are preferred and a low threshold P value threshold is preferred. These are the sequence identity criteria. The tblastn sequence analysis program was used to query a polypeptide sequence against six-way translations of sequences in a nucleotide database. Hits with a P value less than −25, preferably less than −70, and more preferably less than −100, were identified as homologous sequences (exemplary selected sequence criteria). The blastn sequence analysis program was used to query a nucleotide sequence against a nucleotide sequence database. In this case too, higher scores were preferred and a preferred threshold P value was less than −13, preferably less than −50, and more preferably less than −100.

An FVE gene can be isolated via standard PCR amplification techniques. Use of primers to conserved regions of an FVE gene and PCR amplification produces a fragment or full length copy of the desired gene. The PCR template may be DNA, genomic or a cDNA library, or RNA for use with reverse transcriptase PCR(RT-PCR) techniques. Conserved regions can be identified using sequence comparison tools such as BLAST or CLUSTALW for example. Suitable primers have been used and described elsewhere in this application (See Tables 6-12).

Alternatively, a fragment of a sequence from an FVE gene, for example; SEQ ID NO: 1, 3, 5, 6, 7, 9, 11, 13, 15, 17, 19, 21, 23, 25, 27, 29, 31, 33, 35, 206, 216, 220, 239, and 241 is ³²P-radiolabeled by random priming (Sambrook et al., (1989) Molecular Cloning. A Laboratory Manual, 2^{nd} Ed., Cold Spring Harbor Laboratory Press, New York) and used to screen a plant genomic library (the exemplary test polynucleotides). As an example, total plant DNA from Arabidopsis thaliana, Nicotiana tabacum, Lycopersicon pimpinellifolium, Prunus avium, Prunus cerasus, Cucumis sativus, or Oryza sativa are isolated according to Stockinger al (Stockinger, E. J., et al., J. Heredity, 87:214-218 (1996)). Approximately 2 to 10 μg of each DNA sample are restriction digested, transferred to nylon membrane (Micron Separations, Westboro, Mass.) and hybridized. Hybridization conditions are: 42° C. in 50% formamide, 5×SSC, 20 mM phosphate buffer 1×Denhardt's, 10% dextran sulfate, and 100 μg/ml herring sperm DNA. Four low stringency washes at RT in 2×SSC, 0.05% sodium sarcosyl and 0.02% sodium pyrophosphate are performed prior to high stringency washes at 55° C. in 0.2×SSC, 0.05% sodium sarcosyl and 0.01% sodium pyrophosphate. High stringency washes are performed until no counts are detected in the washout according to Walling et al. (Walling, L. L., et al., Nucl. Acids Res. 16:10477-10492 (1988)). Positive isolates are identified, purified and sequenced. Other methods are available for hybridization, for example the ExpressHyb™ hybridization solution available from Clontech.

Identification of FVE Sequence Motifs for use in FVE Gene Inhibition

Another aspect of the invention is selection of sequence motifs to be used for specific down regulation of FVE. The FVE gene comprises six WD-40 motifs. The WD-40 motifs are reported to be commonly found in proteins involved in basic cell regulatory processes. It would therefore be expected that down regulation of a WD-40 motif will affect a large number of non-target proteins and likely result in a plethora of phenotypes if not lethality. In fact, WD40 domains are not absolutely identical and possess sequence flexibility while still meeting the requirements of the domain type. The conserved elements of a WD40 motif are widely spaced and therefore provide a surprising degree of variation in sequence. Use of a WD40 repeat can be used provided that the homology to the target gene motif is high.

A WD40 domain is found in a number of eukaryotic proteins that cover a wide variety of functions including adaptor/regulatory modules in signal transduction, pre-mRNA processing and cytoskeleton assembly. A WD40 domain typically contains a GH dipeptide 11-24 residues from its N-terminus, and the WD dipeptide at its C-terminus which is 40 residues long, hence the name WD40. Between GH and WD lies a conserved core that serves as a stable propeller-like platform to which proteins can bind either stably or reversibly. This conserved core forms a propeller-like structure with several blades where each blade is composed of a four-stranded anti-parallel b-sheet. Instances with few detectable copies are hypothesized to form larger structures by dimerization. Each WD40 sequence repeat forms the first three strands of one blade and the last strand in the next blade. The last C-terminal WD40 repeat completes the blade structure of the first WD40 repeat to create the closed ring propeller-structure. Residues on the top and bottom surface of the propeller are proposed to coordinate interactions with other proteins and/or small ligands. Six copies of the repeat are present in this alignment. Neither the GH dipeptide nor the WD dipeptide is absolutely conserved. When present in a protein, the WD motif is typically found as 4-10 tandem repeats (Smith et al., 1999; and Nocker and Ludwig, 2003).

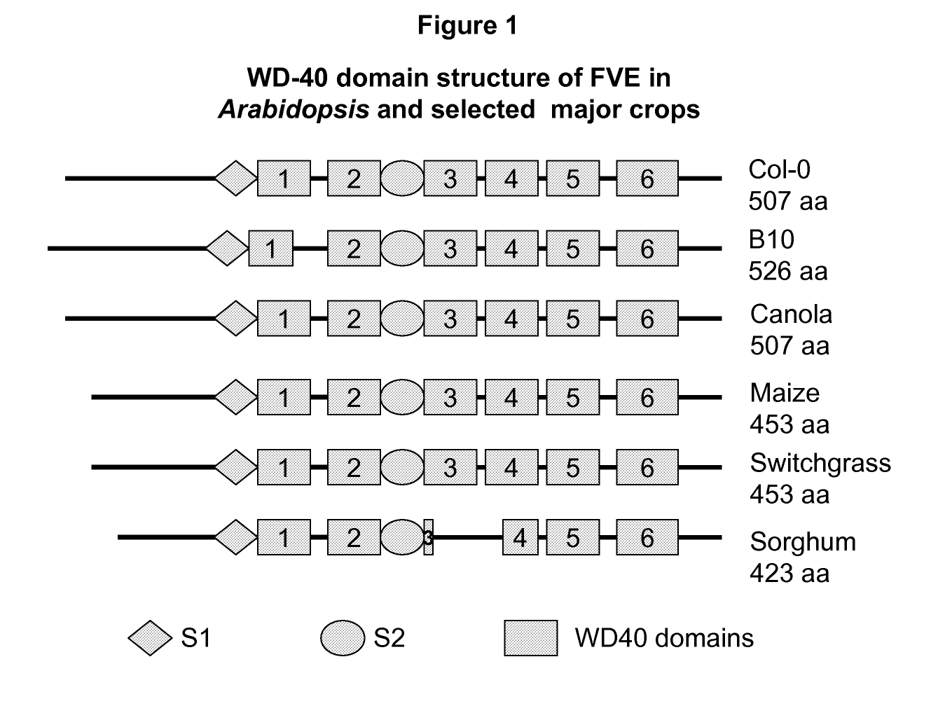

Sequence analysis has identified all six WD40 motifs in AtFVE and multiple alignments of each WD40 motif from various plant species were performed (see FIG. 1). The first WD40 domain (WD40-1) of an FVE gene is found throughout the MSI gene family while the second WD40 domain (WD40-2) appears specifically in FVE (also reported as a MSI4) genes from the species analyzed to date. The remaining four WD40 domains are commonly found in a range of genes and as such require careful assessment for use as targets for specific suppression of FVE expression. Use of these motifs requires assessment as to the likelihood of non-target gene inhibition. For example, use of RNAi technology is highly specific and may be used to effect specific FVE inhibition using these motifs. Antisense and amiRNAi which has less specific inhibition effects necessitate special consideration as to the possibility of non-target effects.

Further sequences analysis shows two specific domains, S1 and S2. The S1 domain is conserved in all known FVE genes from all species, while the S2 domain appears to be a species specific, divergent domain. Each of the S1 and S2 domains and the WD40 domains is suitable for use in FVE inhibition, and useful in methods of producing a plant having increased biomass.

The S1 domain is found as the 36 amino acids preceding the WD40-1 repeat in AtFVE and is identified as SEQ ID NO:41 encoded by SEQ ID NO:57 MOther S1-like regions of an FVE gene may be identified by those skilled in the art that have high homology specifically to an FVE polypeptide as described by SEQ ID NO:37-50, 243 and 244 encoded by the nucleotide sequences as described in SEQ ID NO:51-64, 245 and 246.

Additionally, one skilled in the art may identify additional S2-like regions in an FVE sequence from a species of interest that will be useful in the inhibition of that specific endogenous FVE gene expression and activity. Other S2-like regions of an FVE gene may be identified by those skilled in the art that have high homology specifically to an FVE polypeptide as described by SEQ ID NO:65-78, 223 and 247 encoded by the nucleotide sequences as described in SEQ ID NO:79-92, 233 and 248.

FVE Recombinant Expression Vectors and Host Cells

Another aspect of the invention pertains to vectors, preferably expression vectors, containing a nucleic acid encoding an FVE protein, an FVE gene or genomic sequence or portions thereof and analogs or homologs thereof. As used herein the term expression vector includes vectors which are designed to provide transcription of the nucleic acid sequence. Transcribed sequences may be designed to inhibit the endogenous expression or activity of an endogenous gene activity correlating to the transcribed sequence. Optionally, the transcribed nucleic acid need not be translated but rather inhibits the endogenous gene expression as in antisense or hairpin down-regulation methodology. Alternatively, the transcribed nucleic acid may be translated into a polypeptide or protein product. The polypeptide may be a non-full length, mutant or modified variant of the endogenous protein. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication). Other vectors are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors or plant transformation vectors, binary or otherwise, which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences) or inducible promoters (e.g., induced in response to abiotic factors such as environmental conditions, heat, drought, nutrient status or physiological status of the cell or biotic such as pathogen responsive). Examples of suitable promoters include for example constitutive promoters, ABA inducible promoters, tissue specific promoters and abiotic or biotic inducible promoters. It will be appreciated by those skilled in the art that the design of the expression vector can depend on such factors as the choice of the host cell to be transformed, the level of expression of protein desired as well as timing and location of expression, etc. The expression vectors of the invention can be introduced into host cells to thereby produce proteins or peptides, including fusion proteins or peptides, encoded by nucleic acids as described herein (e.g., FVE proteins, mutant forms of FVE proteins, fusion proteins, etc.).

The recombinant expression vectors of the invention can be designed for expression of FVE genes, FVE proteins, or portions thereof, in prokaryotic or eukaryotic cells. For example, FVE genes or FVE proteins can be expressed in bacterial cells such as Escherichia coli, insect cells (using baculovirus expression vectors), yeast cells, plant cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

In one embodiment, a nucleic acid of the invention is expressed in plants cells using a plant expression vector. Examples of plant expression vectors systems include tumor inducing (Ti) plasmid or portion thereof found in Agrobacterium, cauliflower mosaic virus (CAMV) DNA and vectors such as pBI121.

For expression in plants, the recombinant expression cassette will contain in addition to the FVE nucleic acids, a promoter region that functions in a plant cell, a transcription initiation site (if the coding sequence to transcribed lacks one), and a transcription termination/polyadenylation sequence. The termination/polyadenylation region may be obtained from the same gene as the promoter sequence or may be obtained from different genes. Unique restriction enzyme sites at the 5′ and 3′ ends of the cassette are typically included to allow for easy insertion into a pre-existing vector.

Examples of suitable promoters include promoters from plant viruses such as the 35S promoter from cauliflower mosaic virus (CaMV) (Odell, et al., Nature, 313: 810-812 (1985)), promoters from genes such as rice actin (McElroy, et al., Plant Cell, 163-171 (1990)), ubiquitin (Christensen, et al., Plant Mol. Biol., 12: 619-632 (1992); and Christensen, et al., Plant Mol. Biol., 18: 675-689 (1992)), pEMU (Last, et al., Theor. Appl. Genet., 81: 581-588 (1991)), MAS (Velten, et al., EMBO J., 3: 2723-2730 (1984)), maize H3 histone (Lepetit, et al., Mol. Gen. Genet., 231: 276-285 (1992); and Atanassvoa, et al., Plant Journal, 2(3): 291-300 (1992)), the 5′- or 3′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, ALS promoter, (WO 96/30530), a synthetic promoter, such as Rsyn7, SCP and UCP promoters, ribulose-1,3-diphosphate carboxylase, fruit-specific promoters, heat shock promoters, seed-specific promoters and other transcription initiation regions from various plant genes, for example, including the various opine initiation regions, such as for example, octopine, mannopine, and nopaline. Additional regulatory elements that may be connected to an FVE encoding nucleic acid sequence for expression in plant cells include terminators, polyadenylation sequences, and nucleic acid sequences encoding signal peptides that permit localization within a plant cell or secretion of the protein from the cell. Such regulatory elements and methods for adding or exchanging these elements with the regulatory elements of the FVE gene are known and include, but are not limited to, 3′ termination and/or polyadenylation regions such as those of the Agrobacterium tumefaciens nopaline synthase (nos) gene (Bevan, et al., Nucl. Acids Res., 12: 369-385 (1983)); the potato proteinase inhibitor II (PINII) gene (Keil, et al., Nucl. Acids Res., 14: 5641-5650 (1986); and An, et al., Plant Cell, 1: 115-122 (1989)); and the CaMV 19S gene (Mogen, et al., Plant Cell, 2: 1261-1272 (1990)).

Plant signal sequences which are useful in the invention, include but are not limited to, signal-peptide encoding DNA/RNA sequences which target proteins to the extracellular matrix of the plant cell (Dratewka-Kos, et al., J. Biol. Chem., 264: 4896-4900 (1989)) and the Nicotiana plumbaginifolia extension gene (DeLoose, et al., Gene, 99: 95-100 (1991)), or signal peptides which target proteins to the vacuole like the sweet potato sporamin gene (Matsuka, et al., Proc. Nat'l Acad. Sci. (USA), 88: 834 (1991)) and the barley lectin gene (Wilkins, et al., Plant Cell, 2: 301-313 (1990)), or signals which cause proteins to be secreted such as that of PR1b (Lind, et al., Plant Mol. Biol., 18: 47-53 (1992)), or those which target proteins to the plastids such as that of rapeseed enoyl-ACP reductase (Verwaert, et al., Plant Mol. Biol., 26: 189-202 (1994)).

In another embodiment, the recombinant expression vector is capable of directing expression of the nucleic acid preferentially in a particular cell type (e.g., tissue-specific regulatory elements are used to express the nucleic acid). Tissue-specific regulatory elements are known in the art. Especially useful in connection with the nucleic acids of the present invention are expression systems which are operable in plants. These include systems which are under control of a tissue-specific promoter, as well as those which involve promoters that are operable in all plant tissues.

Organ-specific promoters are also well known. For example, the chalcone synthase-A gene (van der Meer et al., Plant Molecular Biology 15(1):95-109 (1990)) or the dihydroflavonol-4-reductase (dfr) promoter (Elomaa et al., The Plant Journal, 16(1) 93-99) direct expression in specific floral tissues. Also available are the patatin class I promoter which is transcriptionally activated only in the potato tuber and can be used to target gene expression in the tuber (Bevan, M., 1986, Nuc. Acids Res. 14:4625-4636). Another potato-specific promoter is the granule-bound starch synthase (GBSS) promoter (Visser, R. G. R, et al., Plant Mol. Bio. 17:691-699 (1991)).

Other organ-specific promoters appropriate for a desired target organ can be isolated using known procedures. These control sequences are generally associated with genes uniquely expressed in the desired organ. In a typical higher plant, each organ has thousands of mRNAs that are absent from other organ systems (reviewed in Goldberg, P., Trans. R. Soc. London B314:343 (1986)).

The resulting expression system or cassette is ligated into or otherwise constructed to be included in a recombinant vector which is appropriate for plant transformation. The vector may also contain a selectable marker gene by which transformed plant cells can be identified in culture. The marker gene may encode antibiotic resistance. These markers include resistance to G418, hygromycin, bleomycin, kanamycin, and gentamicin. Alternatively the marker gene may encode an herbicide tolerance gene that provides tolerance to glufosinate or glyphosate type herbicides. After transforming the plant cells, those cells having the vector will be identified by their ability to grow on a medium containing the particular antibiotic or herbicide. Replication sequences, of bacterial or viral origin, are generally also included to allow the vector to be cloned in a bacterial or phage host, preferably a broad host range prokaryotic origin of replication is included. A selectable marker for bacteria should also be included to allow selection of bacterial cells bearing the desired construct. Suitable prokaryotic selectable markers also include resistance to antibiotics such as kanamycin or tetracycline.

Other DNA sequences encoding additional functions may also be present in the vector, as is known in the art. For instance, in the case of Agrobacterium transformations, T-DNA sequences will also be included for subsequent transfer to plant chromosomes.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell.

A host cell of the invention, such as a prokaryotic or eukaryotic host cell in culture, can be used to produce (i.e., express) a polypeptide of the invention encoded in an open reading frame of a polynucleotide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

A number of cell types may act as suitable host cell for expression of a polypeptide encoded by an open reading frame in a polynucleotide of the invention. Plant host cells include, for example, plant cells that could function as suitable hosts for the expression of a polynucleotide of the invention include epidermal cells, mesophyll and other ground tissues, and vascular tissues in leaves, stems, floral organs, and roots from a variety of plant species, such as Arabidopsis thaliana, Nicotiana tabacum, Brassica napus, Zea mays, Oryza sativa, Gossypium hirsutu and Glycine max.

Expression of an FVE nucleic acid encoding an altered or mutant FVE protein that is not fully functional can be useful in a dominant-negative inhibition method. An FVE variant polypeptide, or portion thereof, is expressed in a plant such that it has partial functionality. The variant polypeptide may for example have the ability to bind other molecules but does not permit proper activity of the complex, resulting in overall inhibition of FVE activity.

### Transformed Plants Cells and Transgenic Plants

The invention includes a protoplast, plants cell, plant tissue and plant (e.g., monocot or dicot) transformed with an FVE nucleic acid, a vector containing an FVE nucleic acid or an expression vector containing an FVE nucleic acid. As used herein, “plant” is meant to include not only a whole plant but also a portion thereof (i.e., cells, and tissues, including for example, leaves, stems, shoots, roots, flowers, fruits and seeds).

The plant can be any plant type including, but not limited to, for example, species from the genera Arabidopsis, Brassica, Oryza, Zea, Sorghum, Gossypium, Triticum, Glycine, Pisum, Phaseolus, Lycopersicon, Trifolium, Cannabis, Cucurbita, Rosa, Vitis, Juglans, Fragaria, Lotus, Medicago, Onobrychis, Trigonella, Vigna, Citrus, Linum, Geranium, Manihot, Daucus, Raphanus, Sinapis, Atropa, Capsicum, Datura, Hyoscyamus, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Lolium, Avena, Hordeum, Secale, Picea, Caco, Solanaceae, Panicum, Brachypodium, Silene, Physcomitrella, Miscanthus, Jatropha, Salix, Andropogon, Saccharum, Camelina, Eucalyptus, Arundo and Populus.

The invention also includes cells, tissues, including for example, leaves, stems, shoots, roots, flowers, fruits and seeds and the progeny derived from the transformed plant.

Numerous methods for introducing foreign genes into plants are known and can be used to insert a gene into a plant host, including biological and physical plant transformation protocols (See, for example, Miki et al., “Procedure for Introducing Foreign DNA into Plants”, In: Methods in Plant Molecular Biology and Biotechnology, Glick and Thompson, eds., CRC Press, Inc., Boca Raton, pages 67-88 (1993); and Andrew Bent in, Clough S J and Bent A F, “Floral dipping: a simplified method for Agrobacterium-mediated transformation of Arabidopsis thaliana” (1998)). The methods chosen vary with the host plant, and include chemical transfection methods such as calcium phosphate, polyethylene glycol (PEG) transformation, microorganism-mediated gene transfer such as Agrobacterium (Horsch, et al., Science, 227: 1229-31 (1985)), electroporation, protoplast transformation, micro-injection, flower dipping and biolistic bombardment.

### Agrobacterium-Mediated Transformation

The most widely utilized method for introducing an expression vector into plants is based on the natural transformation system of Agrobacterium tumefaciens and A. rhizogenes which are plant pathogenic bacteria which genetically transform plant cells. The Ti and Ri plasmids of A. tumefaciens and A. rhizogenes, respectfully, carry genes responsible for genetic transformation of plants (See, for example, Kado, Crit. Rev. Plant Sci., 10:1-32 (1991)). Descriptions of the Agrobacterium vector systems and methods for Agrobacterium-mediated gene transfer are provided in Gruber et al., supra; and Moloney, et al, Plant Cell Reports, 8: 238-242 (1989).

Transgenic Arabidopsis plants can be produced easily by the method of dipping flowering plants into an Agrobacterium culture, based on the method of Andrew Bent in, Clough S J and Bent A F, “Floral dipping: a simplified method for Agrobacterium-mediated transformation of Arabidopsis thaliana” (1998). Wild-type plants are grown until the plant has both developing flowers and open flowers. The plants are inverted for 1 minute into a solution of Agrobacterium culture carrying the appropriate gene construct. Plants are then left horizontal in a tray and kept covered for two days to maintain humidity and then righted and bagged to continue growth and seed development. Mature seed is bulk harvested.

### Direct Gene Transfer

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes. (Sanford, et al., Part. Sci. Technol., 5: 27-37 (1987); Sanford, Trends Biotech, 6: 299-302 (1988); Sanford, Physiol. Plant, 79: 206-209 (1990); Klein, et al., Biotechnology, 10: 286-291 (1992)).

Plant transformation can also be achieved by the Aerosol Beam Injector (ABI) method as described in U.S. Pat. No. 5,240,842, and U.S. Pat. No. 6,809,232. Aerosol beam technology is used to accelerate wet or dry particles to speeds enabling the particles to penetrate living cells. Aerosol beam technology employs the jet expansion of an inert gas as it passes from a region of higher gas pressure to a region of lower gas pressure through a small orifice. The expanding gas accelerates aerosol droplets, containing nucleic acid molecules to be introduced into a cell or tissue. The accelerated particles are positioned to impact a preferred target, for example a plant cell. The particles are constructed as droplets of a sufficiently small size so that the cell survives the penetration. The transformed cell or tissue is grown to produce a plant by standard techniques known to those in the applicable art.

### Regeneration of Transformants

The development or regeneration of plants from either single plant protoplasts or various explants is well known in the art (Weissbach and Weissbach, 1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a polypeptide of interest introduced by Agrobacterium from leaf explants can be achieved by methods well known in the art such as described in Horsch et al., (1985). In this procedure, transformants are cultured in the presence of a selection agent and in a medium that induces the regeneration of shoots in the plant strain being transformed as described in Fraley et al. (1983). In particular, U.S. Pat. No. 5,349,124 details the creation of genetically transformed lettuce cells and plants resulting therefrom which express hybrid crystal proteins conferring insecticidal activity against Lepidopteran larvae to such plants.

This procedure typically produces shoots within two to four months and those shoots are then transferred to an appropriate root-inducing medium containing the selective agent and an antibiotic to prevent bacterial growth. Shoots that rooted in the presence of the selective agent to form plantlets are then transplanted to soil or other media to allow the production of roots. These procedures vary depending upon the particular plant strain employed, such variations being well known in the art.

Preferably, the regenerated plants are self-pollinated to provide homozygous transgenic plants, or pollen obtained from the regenerated plants is crossed to seed-grown plants of agronomically important, preferably inbred lines. Conversely, pollen from plants of those important lines is used to pollinate regenerated plants. A transgenic plant of the present invention containing a desired polypeptide is cultivated using methods well known to one skilled in the art.

A preferred transgenic plant is an independent segregate and can transmit the FVE gene construct to its progeny. A more preferred transgenic plant is homozygous for the gene construct, and transmits that gene construct to all offspring on sexual mating. Seed from a transgenic plant may be grown in the field or greenhouse, and resulting sexually mature transgenic plants are self-pollinated to generate true breeding plants. The progeny from these plants become true breeding lines that are evaluated for decreased expression of the FVE gene.

### Method of Producing Transgenic Plants

Also included in the invention are methods of producing a transgenic plant having increased biomass. The method includes introducing into one or more plant cells a compound that inhibits FVE expression or activity in the plant to generate a transgenic plant cell and regenerating a transgenic plant from the transgenic cell. The compound can be, e.g., (i) an FVE polypeptide; (ii) an FVE nucleic acid, analog, homolog, portion or variant thereof, (iii) a nucleic acid that decreases expression of an FVE nucleic acid. A nucleic acid that decreases expression of an FVE nucleic acid may include promoters or enhancer elements. The FVE nucleic acid can be either endogenous or exogenous, for example an Arabidopsis FVE nucleic acid may be introduced into a Brassica or corn species. For example the compound comprises the nucleic acid sequence of SEQ ID NO: 1, 3, 5, 6, 7, 9, 11, 13, 15, 17, 19, 21, 23, 25, 27, 29, 31, 33, 35, 206, 216, 220, 239, and 241 or a portion thereof, such as SEQ ID NO:51-64, 245, 246, 79-92, 233, 248, 107-120, 234, 252, 135-148, 235, 250, 163-176, 256, 254, 273-288, 305-320 and 337-352 or a expression cassette such as SEQ ID NO:372-379. Preferably, the compound is an FVE nucleic acid sequence endogenous to the species being transformed. Alternatively, the compound is an FVE nucleic acid sequence exogenous to the species being transformed and having at least 70% homology to the endogenous target sequence.

In various aspects the transgenic plant has an altered phenotype as compared to a wild-type plant (i.e., untransformed). By altered phenotype is meant that the plant has a one or more characteristic that is different from the wild-type plant. For example, when the transgenic plant has been contacted with a compound that decreases the expression or activity of an FVE nucleic acid, the plant has a phenotype such as increased biomass as compared to a wild-type plant.

The plant can be any plant type including, but not limited to, for example, species from the genera Arabidopsis, Brassica, Oryza, Zea, Sorghum, Gossypium, Triticum, Glycine, Pisum, Phaseolus, Lycopersicon, Trifolium, Cannabis, Cucurbita, Rosa, Vitis, Juglans, Fragaria, Lotus, Medicago, Onobrychis, Trigonella, Vigna, Citrus, Linum, Geranium, Manihot, Daucus, Raphanus, Sinapis, Atropa, Capsicum, Datura, Hyoscyamus, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Lolium, Avena, Hordeum, Secale, Picea, Caco, Solanaceae, Panicum Brachypodium, Silene, Physcomitrella, Miscanthus, Jatropha, Salix, Andropogon, Saccharum, Camelina, Eucalyptus, Arundo and Populus.

The invention will be further illustrated in the following non-limiting examples.

### EXAMPLES

### Example 1

### Identification of a Plant Having Increased Biomass

A mutagenized population of Arabidopsis thaliana var. Columbia (Col-0) was screened and the Arabidopsis fve-b10 mutant was identified as having beneficial phenotypes including increased biomass relative to a wild-type control, and a delay in the time to onset of flowering.

The fve-b10 mutant was used to cross with another Arabidopsis wild-type accession Landsberg erecta (Ler). The true FIs of Ler×fve-b10 were identified with simple-sequence length polymorphisms (SSLP) markers. An F2 population of 594 individuals were prepared for gene mapping. Rough mapping results based on Bulked Segregants Analysis (BSA) using 33 markers from Chromosome I, II, III, IV, and V, showed that the biomass enhancement trait (BET) was located between molecular markers of RGA and PLS3 on Chromosome II. The interval between RGA and PLS3 is about 8.75 mega base pairs (Mb).

Fine mapping was conducted using a total of 594 F2 individuals. Based on literature, a total of 61 genes involved in different pathways of flowering time were investigated for their phenotypes and genetic location. These genes are involved in flowering time of the long day pathway, autonomous pathway, vernalization pathway and gibberellin pathway, as well as floral pathway integrators and floral meristem identity.

Among the 61 flowering genes investigated, only two of them were located within the 8.75 Mb interval between RAG and PLS3. The At2g19520 (FVE) mutant displayed a delayed flowering phenotype, therefore At2g19520 (FVE) was selected as the gene candidate responsible for the fve-b10 gene. Genomic fragments of FVE from both Col-0 and fve-b10 were amplified and purified for sequencing.

A total of 3,763 bp of genomic DNA of the FVE locus from fve-b10 was successfully sequenced, including 346 bp upstream of the start codon of FVE, 3217 bp between the start codon and stop codon, and 200 bp downstream of the stop codon. The sequencing results revealed that compared with wild-type FVE in Col-0, fve-b10 has a 13 bp deletion at the junction point of the 5^{th} intron and 6^{th} exon, which changes the open reading frame (ORF) of the FVE gene in fve-b10. Further studies revealed that a longer FVE ORF-fve-b10 (1581 bp) encoding 526 amino acids was produced in fve-b10, while the wild-type FVE ORF has 1524 bp encoding 507 amino acids. Protein tertiary structure analysis showed that fve-b10 has two extra β-sheets as compared to Wt-FVE.

These results, combined with complementation test results therefore confirm that FVE is the gene responsive for the biomass enhancement trait (BET).

### Example 2

### Physiological Characterization of the fve-b10 Mutant

i) fve-b10 Mutant has Greatly Enhanced Biomass, Chlorophyll Content and Seed Yield

Arabidopsis fve-b10 mutant plants and wild-type (WT) controls were grown in 3″ pots (1 plant/pot) in soil-less mix (Sunshine) in a growth chamber under optimal conditions (22° C., 70% RH, 18 hr light) either at an optimal light of 200 uE or low light of 80 uE. Plants were harvested at developmental stages including vegetative, bolting and mid-flowering stage, and total shoot biomass, as well as fresh and dry weights of stems and leaves, were determined. The enhanced shoot biomass of fve-b10 mutant was the most pronounced during the mid-flowering stage. For fve-b10 plants grown at optimal light (200uE), biomass enhancement over the WT plants was 2 fold for stem dry weight (DW), 6 fold for leaf DW (where twice as many leaves and larger leaves were seen), and stems were twice as thick in fve-b10 as compared to WT. Although the leaf size was larger than wild-type the cell size was unchanged relative to WT. The flowering time of the fve-b10 mutant was delayed by 8 to 13 days.

In plants grown at low light (80 uE) the biomass enhancement of the fve-b10 mutant was more apparent. Stem DW was 3 fold higher (more stems and thicker stems) and leaf DW was 26 fold higher (5 times more leaves and larger leaves) than in WT. A summary of the biomass enhancement for fve-b10 mutant plants as compared to WT plants grown under optimal light and low light conditions is shown in Table 1 below.

Analysis of stem cross-sections was performed using a dissection microscope (Leica) to determine stem thickness measurements and structural observations of cell organization. Stem cross-sections were obtained from the main stem taken at 3 to 4 cm above the soil when plants were 3 days post-first flower.

Stems of fve-b10 mutant plants were twice as thick as WT plants (2.4 mm vs 1.2 mm diameter) and had twice as many vascular bundles per cross section (14 vs 6) as WT. In addition, the inter-vascular layer of fiber was 2 fold thicker in fve-b10 stems. The chlorophyll content of the leaves from plants grown both under optimal and low light was 40% higher in fve-b10 mutant than WT plants, but there was no difference in chlorophyll a to b ratio between the mutant and the WT.

TABLE 1
Biomass enhancement of fve-b10 mutant over WT at low or optimal light.

| | Low light (80uE) | | Optimal light (200 uE) | |
|---|---|---|---|---|
| entry | Leaf DW (mg) | Stem DW (mg) | Leaf DW (mg) | Stem DW (mg) |
| fve- | 340 ± 28 | 102 ± 10 | 630 ± 35 | 430 ± 20 |
| b10 | | | | |
| WT | 13 ± 2 | 33 ± 3 | 95 ± 6 | 210 ± 18 |

Plants grown under optimal light and in 4 inch and 6 inch pots were grown to maturity for final biomass and seed yield harvests (n=8 to 14). The shoot biomass of plants grown to maturity in 6″ pots under optimal conditions was 81% greater in the fve-b10 mutant and the final seed yield was 15% greater than that of WT. When plants were grown in smaller pots (4″) under optimal conditions the seed yield of the fve-b10 mutant ranged from 20% to 46% greater than WT. The harvest index (ratio of seed yield to the total shoot biomass, including seeds) was lower in fve-b10 than WT. Without intending to be bound by any theory, these results are indicative of more of the plant resources going towards shoot biomass (mostly stems) than seed production. A summary of the shoot biomass and seed yield for fve-b10 mutant plants grown to maturity in 6″ pots as compared to WT plants is shown in Table 2 below.

TABLE 2
Total shoot biomass and seed yield of mature plants grown in 6” pots.

| entry | Total shoot DW (g) | Seed yield/plant (g) | Harvest index |
|---|---|---|---|
| fve-b10 | 15.8 ± 0.3 | 5.2 ± 0.2 | 0.33 |
| WT | 10.2 ± 0.5 | 4.4 ± 0.2 | 0.43 |

The fve-b10 mutant and WT Arabidopsis plants were grown in magenta boxes containing 100 ml of solid agar media enriched with 0.5 MS nutrients for optimal growth. Plants were grown in a growth chamber at 22° C., 17 hr light (200 uE) until four days into flowering. Plants were harvested at that time for shoot and root dry biomass determinations. The results showed that fve-b10 mutant plants had both, shoot and root dry biomass enhanced as compared to WT controls. These were 2.1 and 2.4 fold greater in the fve-b10 mutant than in WT, respectively. This data indicates that the biomass enhancement in the fve-b10 mutant is present in the whole plant.

ii) fve-b10 Mutant Has More Efficient Photosynthesis at Lower Lights and Greater Water Use Efficiency Under Optimal and Water Limited Conditions.

Plants were grown one per 4″ pot under optimal conditions in a growth chamber as described above. Gas exchange measurements were done on the two youngest fully developed leaves per plant 7 days after first open flower. Fourteen replicate plants per entry were measured. Gas exchange measurements were done in the growth chamber using the Li-6400 (LiCor) and a built in red light source set at 200 uE (light levels of the growth conditions). Ambient CO₂ concentration at the leaves was 400 ppm as set by Li-6400.

Specific average rate of photosynthesis under optimal conditions in μmol CO₂ per m of a leaf per min was 15% lower in fve-b10 than in WT (8.0±0.2 and 9.6±0.2, respectively). The rate of transpiration in mmol water per m per second was 48% lower in fve-b10 as compared to that of control WT (2.2±0.1 vs. 4.2±0.1, respectively). Water use efficiency (WUE) was calculated as the ratio of photosynthesis to transpiration and was significantly greater (63%) in the fve-b10 mutant as compared to the control plants under optimal conditions.

The fve-b10 mutant and WT control were used for measurements of maximum rates of photosynthesis at saturating light levels. From measurements of CO₂ exchange rates at very low light levels the light compensation point was determined (light level where the net CO₂ exchange rate is zero, or where rate of respiration equals rate of photosynthesis). These were done on youngest fully developed leaves using the Li-6400. A lower light compensation point is indicative of a plant having greater ability to utilize low light levels. The results of these measurements showed that the maximum rates of photosynthesis at saturating light were not significantly different between fve-b10 and WT (12±1.1 and 14±0.2 umol CO₂/m²/s, respectively). The saturating light levels were also not different between fve-b10 and WT (around 900 uE). However, the light compensation points was significantly lower (8.3±0.6 uE) in fve-b10 than in WT (12.7±1.5 uE), indicating that fve-b10 is able to utilize lower light intensities for photosynthesis better than the control (WT) but still have functional photosynthetic apparatus at the same high levels as controls. This data is in accordance with the results of biomass accumulation under low light (80 uE) growth conditions, where fve-b10 mutant had greater enhancement of biomass over WT than at optimal light levels (around 200 uE).

Water use efficiency (WUE) was also measured gravimetrically on plants grown in 3″ pots at a density of 5 plants per pot, under optimal conditions in a growth chamber as previously described. Plants were watered daily with a finite amount of water and the daily water loss from the pots was measured by weighing the pots. The shoot biomass was harvested on days 0, 2 and 4 of treatment, with day 0 being the first day of flower opening. A second group of plants were grown under drought stress conditions by watering with a finite amount of water on day 0 and cessation of subsequent watering. Drought stress conditions were achieved within 2 to 3 days. Pots were weighed daily to determine the daily water loss. These plants were harvested on day 4 of treatment. WUE was calculated from the ratio of biomass accumulated and the amount of water lost between days 0 and 4 for both optimal and drought treatments. The mutant fve-b10 had 82% greater WUE than control under optimal conditions (2.49 vs 1.37) and 51% greater WUE under drought conditions (2.37 vs 1.57) than the control WT. This data supports the gas exchange data described above that the mutant fve-b10 plants use water more efficiently to accumulate shoot biomass.

The growth rates in grams of shoot dry weight per day were calculated in plants maintained under water limited conditions for 7 days and the growth rate of fve-b10 mutant plants was 6 fold higher (0.101 g/day) than that of the control WT (0.018 g/day).

iii) Upon Vernalization of The Seeds, Biomass Enhancement and Water Use Efficiency is not Dependent Upon Delay of Flowering

Seeds of fve-b10 mutant and WT were placed on agar plates containing ½ MS media at 4° C. for 7 weeks to vernalize (cold exposure). The first two weeks were in darkness and once seeds germinated the young seedlings were placed in a chamber at 4° C. at low light intensity (150 uE) with an 18 hr photoperiod for the remaining 5 weeks. After 7 weeks of vernalization plates were transferred to 22° C. and allowed to grow for 5 days, after which young seedlings were transplanted into 3″ pots containing soilless mix (sunshine mix #1). Five plants per pot were grown under optimal conditions in a growth chamber until first day of flowering. Optimal and drought treatments were started (day 0) by watering up all the plants with the same amount of water. In the optimal group of plants, water was replenished daily whereas the drought group received no further water. Daily water loss was measured from all plants and plants were harvested on days 0, 2 and 4 of treatment.

The results of this experiment showed that fve-b10 plants following vernalization flowered at the same time as the WT plant (17 days after the end of vernalization). These mutant plants still showed enhanced leaf biomass as compared to WT especially under optimal conditions (25% to 51% greater). The water loss in the first two days of the treatment under optimal conditions was 14% lower in the fve-b10 mutant and the water use efficiency in those two days of the treatment was 58% and 22% greater in the fve-b10 mutant as compared to WT at optimal and drought conditions, respectively. The difference in WUE was associated with greater biomass accumulation in the fve-b10 mutant.

iv) fve-b10 Plants Are More Resistant to Oxidative Stress, Heat Stress and Show Reduced Nitrogen (N.) Requirements

Plants were grown, one per 3″ pot, under optimal conditions in a growth chamber. Three leaf disks were removed from the three youngest fully developed leaves of 17 day old plants. Leaf disks were placed on filter paper wet with 5 μM paraquat (PQ) (methylviologen) under a light intensity of 100 uE for 32 hours. Treatment resulted in oxidative damage to chlorophyll and bleaching of the leaves. After 32 hours the leaf disks were frozen in liquid nitrogen and extracted in methanol for assays of total chlorophyll. The extent of chlorophyll oxidation is associated with susceptibility to oxidative stress. The results shown in Table 3 indicate that chlorophyll content of fve-b10 leaves under optimal conditions was 30% greater than that of WT. However, after paraquat treatment, which reduced the total chlorophyll by up to 80%, fve-b10 leaf disks had 92% greater chlorophyll content than WT, indicating greater resistance to oxidative stress in fve-b10 mutant.

TABLE 3
Chlorophyll content per leaf area following oxidative stress

| | | | PQ: Total |
|---|---|---|---|
| | Optimal: Total | PQ: Total | chlorophyll |
| entry | chlorophyll (mg/m²) | chlorophyll (mg/m²) | (% optimal) |
| fve-b10 | 389 ± 11 | 121 ± 12 | 31% |
| WT | 299 ± 7 | 63 ± 11 | 21% |

Growth under heat stress conditions was evaluated as follows, fve-b10 and WT plants were grown three per 3″ pot for ten days under optimal conditions in short day light conditions of 10 hr to promote vegetative growth. Half of the plants were then exposed to a heat treatment of 4 hr per day at 40° C. for 3 days and on day four plants were heated to 43° C. for 4 hr. Three days following heat treatment the heated and optimal groups of plants were harvested for shoot biomass determinations (n=8 per entry and per treatment). The results (Table 4) showed that fve-b10 mutant plants were 9% less inhibited in their growth as a result of the heat treatments, indicating resistance to heat stress at early vegetative stage of growth.

TABLE 4
Shoot dry weight following 4 days of heat
stress at vegetative growth stage.

| | Optimal | Heat | Heat |
|---|---|---|---|
| Entry | shoot DW (mg) | Shoot DW (mg) | shoot DW (% opt) |
| fve-b10 | 61 ± 1 | 43 ± 1 | 70% |
| WT | 58 ± 2 | 35 ± 1 | 61% |

Growth under low nitrogen conditions was evaluated as follows. Seedlings of fve-b10 and WT were grown on agar plates (10 seedlings per plate) containing optimal growth media (½ MS with 20 mM N.) or media deficient in N. (0.5 mM N) for 14 days. These plates were kept in a vertical position under optimal conditions in a growth chamber (22° C., 18 hr light of 200 uE). After 14 days of growth, 8 seedlings were harvested for shoot biomass determinations and 2 seedlings were harvested for shoot chlorophyll content determinations. The results showed that growth on low N. resulted in 45% lower seedling shoot dry weight in the fve-b10 mutant, and 48% lower seedling shoot dry weight in WT, as compared to plants grown under optimal conditions. This result suggests that the fve-b10 mutant plants at early seedling growth do not require more N. for their growth than WT plants and in fact may be more efficient at nitrogen usage.

### Example 3

### Identification of the fve-b10 and Wild-Type FVE Genes

Map-based cloning was utilized to localize the mutation and identify the gene. Total RNA was isolated from fve-b10 and Col-O and used as template for RT-PCR using the primer pair SEQ ID NO:197 and 198 to produce full length cDNAs of At fve-b10 (SEQ ID NO:1) and At FVE (SEQ ID NO:3). PCR products were isolated, cloned and sequenced.

Sequencing results show that the isolated full coding region of the wild-type Col-0 AtFVE is identical to the coding region of At2g19520 in the TAIR database. The genomic sequence of the mutant A fve-b10 has a 13 nucleotide deletion at the junction point of the 5th intron and 6th exon as compared with wild-type AtFVE. The altered open reading frame of the Atfve-b10 results in an altered coding sequence having an additional 57 nucleotides encoding an additional 19 amino acids.

| Genomic Sequence Alignment: Intron sequence is underlined. | | |
|---|---|---|
| fve-b10 | CTCACACCGACAGTCCTGATGTGAGTGCTGCTTCTATTTTGTTATGGTCATAGCAACTTG | 1680 |
| WT-FVE | CTCACACCGACAGTCCTGATGTGAGTGCTGCTTCTATTTTGTTATGGTCATAGCAACTTG | 1680 |
| | ************************************************************ | |
| fve-b10 | AAATATGTCGGTTTCATATTTCTGTATTTGGCAGTCAAAGAGCATCCTTTGTTCGGACAT | 1740 |
| WT-FVE | AAATATGTCGGTTTCATATTTCTGTATTTGGCAGTCAAAGAGCATCCTTTGTTCGGACAT | 1740 |
| | ************************************************************ | |
| fve-b10 | ATGTCCAGTTTCAGAGTTATCTAAATACAATATGTC-------------CAGTTTGGATG | 1787 |
| WT-FVE | ATGTCCAGTTTCAGAGTTATCTAAATACAATATGTTGATTTCAGGTTCTCATTTGGGATG | 1800 |
| | *********************************** ** ** ***** | |
| cDNA sequence alignment | | |
| fve-b10 | GCTACTCACACCGACAGTCCTGATTCAAAGAGCATCCTTTGTTCGGACATATGTCCAGTT | 600 |
| WT-FVE | GCTACTCACACCGACAGTCCTGAT------------------------------------ | 568 |
| | ************************ | |
| fve-b10 | TCAGAGTTATCTAAATACAATATGTCCAGTTTGGATGTTGAAACCCAACCAAACCGTCAT | 660 |
| WT-FVE | ---------------------GTTCTCATTTGGGATGTTGAAACCCAACCAAACCGTCAT | 603 |
| | * ** ** **************************** | |
| polypeptide sequence alignment | | |
| fve-b10 | ATHTDSPDSKSILCSDICPVSELSKYNMSSLDVETQPNRHAVLGAANSRPDLILTGHQDN | 240 |
| WT-FVE | ATHTDSPD-------------------VLIWDVETQPNRHAVLGAANSRPDLILTGHQDN | 221 |
| | ******** : ***************************** | |

### Confirmation of FVE Gene by Complementation

Expression of a wild-type FVE gene in the mutant background should restore the wild-type growth phenotype and verify that the FVE gene is the desired target gene. A transgenic complementation test was done using a constitutive promoter to drive ectopic expression of At2g19520 (FVE) from Arabidopsis ecotype Columbia (Col-O) in the Atfve-b10 background containing the mutant allele.

A full length coding sequence of wild-type (Col-0) FVE was PCR amplified using the primer pair according to SEQ ID NOS: 197 and 198, and cloned into a binary vector pBI121AGUS-Basta using the restriction sites of Xba I and BamH I. The construct (35S-AtFVE) was transformed into the Arabidopsis fve-b10 mutant by Agrobacterium-mediated transformation. The presence of the transgene was verified in each of the T1, T2 and T3 generations, each of which had a growth phenotype like a wild-type plant.

Western blotting was conducted to determine the association between expression levels of FVE in fve-b10 and complementation grades of biomass enhancement. Inhibited expression of FVE is correlated with phenotypes such as increased biomass and confirms the role of FVE.

Over-Expression of the AtFVE Gene in the Arabidopsis fve-b10 Mutant Background

Eleven transgenic lines and two nulls of ³⁵S-AtFVE transformed into the fve-b10 mutant plant background plus the control lines of the fve-b10 mutant and a WT Arabidopsis (Col) were grown in a growth study. The results of this study showed that over-expression of a wild-type FVE gene in the fve-b10 mutant background resulted in a gene rescue and reversion of the fve-b10 mutant back towards wild-type-like phenotypes. The rescue of the mutant was not complete as indicated by some biomass accumulation and delay of flowering relative to the WT controls however the spectrum was reverted significantly from the fve-b10 phenotype. These results are shown in Table 5 below.

TABLE 5
Reversion of fve-b10 towards Wild-type by 35S-AtFVE construct

| | Days | Stem | | | Shoot | Shoot |
|---|---|---|---|---|---|---|
| | to | DW | Rosette | Leaf DW | DW | DW (% |
| entry | flower | (mg) | leaf # | (mg) | (mg) | of WT) |
| fve-b10 | 34 | 298 ± 30 | 98 | 983 ± 26 | 1280± | 377% |
| 60-1 | 31 | 310 ± 21 | 38 | 465 ± 19 | 775± | 228% |
| 65-4 | 27 | 279 ± 19 | 46 | 423 ± 25 | 702± | 207% |
| 51-3 | 25 | 291 ± 15 | 43 | 335 ± 21 | 626± | 184% |
| 37-4 | 26 | 281 ± 15 | 43 | 333 ± 30 | 614± | 181% |
| 76-11 | 25 | 295 ± 23 | 41 | 295 ± 18 | 590± | 174% |
| 95-1 | 25 | 242 ± 16 | 47 | 332 ± 15 | 574± | 169% |
| 75-2 | 24 | 278 ± 20 | 39 | 260 ± 15 | 538± | 158% |
| 103-5 | 25 | 266 ± 16 | 37 | 270 ± 9 | 536± | 158% |
| 99-6 | 25 | 234 ± 13 | 39 | 300 ± 27 | 534± | 157% |
| 105-7 | 25 | 258 ± 16 | 37 | 265 ± 14 | 523± | 154% |
| 100-9 | 24 | 216 ± 26 | 34 | 204 ± 12 | 420± | 124% |
| 105-5 | 32 | 301 ± 30 | 82 | 958 ± 66 | 1259± | 370% |
| (null) | | | | | | |
| 37-2 | 34 | 268 ± 19 | 83 | 1000 ± 73 | 1268± | 373% |
| (null) | | | | | | |
| WT | 22 | 155 ± 15 | 33 | 185 ± 13 | 340± | 100% |

### Example 4

### Identification of FVE from Plants

An FVE gene can be identified by selecting a gene that has high homology to an FVE sequence, for example SEQ ID NOs: 1, 3, 5, 6, 7, 9, 11, 13, 15, 17, 19, 21, 23, 25, 27, 29, 31, 33, 35, 206, 216, 220, 239, and 241. By high homology it is meant that a putative FVE nucleic acid sequence exhibits a degree of identity to a known FVE sequence of preferably at least 70%, 75%, 80%, 85%, 90%, 95%, 98%, or 99% identity. Further, an FVE gene is identified through analysis of candidate genes that share conformational protein structure. Such structural motifs, such as WD40, S1-like or S2-like motifs, may assist in identification of related proteins and their structure.

Functional Analysis of FVE Homologs from Major Crops
i) Brassica napus

A Brassica napus, BnFVE, sequence was isolated and ectopically expressed in the Arabidopsis fve-b10 background thereby demonstrating the functional identity of the BnFVE sequence as being functionally equivalent to AtFVE. The 5′-end and the 3′-end of the cDNA were obtained using the primer pair SEQ ID NOs:201 and 202 for 5′ RACE and by using the primer SEQ ID NOs:201 and 203 in 3′-RACE. The resulting 5′-RACE product and 3′-RACE product of BnFVE was purified and sequenced. Based on sequencing results, the primer pair SEQ ID NOs:204 and 205 were designed and synthesized to amplify a full length coding sequence for FVE from Brassica by RT-PCR. The resulting sequence is disclosed as SEQ ID NO:206. The BnFVE open reading frame is 1524 nucleotides in length and encodes for 507 amino acids.

The BnFVE gene was cloned into a binary vector such as pBI121-Basta using restriction sites of Xba I and Sac I. The resulting construct of ³⁵S-BnFVE was transformed into fve-b10 mutant plant by Agrobacterium-mediated transformation.

The presence of the transgene was confirmed by PCR analysis and selection by Basta resistance. Transgenic T 1 plants of fve-b10::35 S-BnFVE display a wild-type phenotype thereby demonstrating that the BnFVE gene is functionally equivalent to the AtFVE gene.

ii) Zea mays

Two Zea mays, ZmFVE, sequences were isolated and ectopically expressed in the Arabidopsis fve-b10 background thereby demonstrating the functional identity of the ZmFVE sequences as being functionally equivalent to AtFVE. The ZmFVE genes were RT-PCR amplified using the primer pairs SEQ ID NOs:208 and 209 or SEQ ID NOs:210 and 211, then subsequently cloned and sequenced. Full length cDNAs were obtained and referred to as ZmFVE1 (SEQ ID NO: 220) and ZmFVE2 (SEQ ID NO:23). Each have an open reading frame of 1362 nucleotides, encode a 453 amino acid polypeptide, and are 98% identical. Compared to the Arabidopsis sequences the maize nucleotide sequences are 74% identical and the amino acid sequences are 77% identical.

The ZmFVE genes were cloned into a binary vector such as the pBI121-Basta using restriction sites of Xba I and Sac I. The resulting constructs of ³⁵S-ZmFVE and ³⁵S-ZmFVE were transformed into fve-b10 by Agrobacterium-mediated transformation.

The presence of the transgene was confirmed by PCR analysis and selection by Basta resistance. Transgenic plants of fve-b10::35 S-ZmFVEJ and fve-b10::35S-ZmFVE2 display a wild-type phenotype thereby demonstrating that the ZmFVE1 and ZmFVE2 genes are functionally equivalent to the AtFVE gene.

iii) Panicum virgatum (Switchgrass)

A Panicum virgatum (switchgrass), PvFVE, sequence was isolated and ectopically expressed in the Arabidopsis fve-b10 background thereby demonstrating the functional identity of the PvFVE sequence as being functionally equivalent to AtFVE. A partial PvFVE gene was amplified by RT-PCR using the primer pair SEQ ID NOs:212 and 213. The RT-PCR products were cloned and sequenced. A full length cDNA was subsequently obtained by using the primer pair SEQ ID NOs:214 and 215 for PCR and cloning the resulting product. The resulting sequence is disclosed as SEQ ID NO:216. The switchgrass FVE gene (PvFVE) has an open reading frame of 1362 nucleotides in length and encodes for 453 amino acids. The nucleotide sequence is 92% identical to maize FVE1 and the amino acid sequence 97% identical to maize FVE1.

The PvFVE gene was cloned into a binary vector such as pBI121-Basta using restriction sites of Xba I and Sac I. The resulting construct of ³⁵S-PvFVE was transformed into fve-b10 by Agrobacterium-mediated transformation. The presence of the transgene is confirmed by PCR analysis and selection by Basta resistance. Transgenic T1 plants of fve-b10::35S-PvFVE are shown to have a wild-type phenotype thereby demonstrating that the PvFVE gene is functionally equivalent to the AtFVE gene. iv) Glycine max (soybean) A Glycine max (soybean), GmFVE, sequence was isolated and ectopically expressed in the Arabidopsis fve-b10 background thereby demonstrating the functional identity of the GmFVE sequence as being functionally equivalent to AtFVE. A full length cDNA was obtained by using the primer pair SEQ ID NOs:218 and 219 for RT-PCR and cloning the resulting product. The resulting sequence is disclosed as SEQ ID NO:9. The soybean FVE gene (GmFVE) has an open reading frame of 1542 nucleotides in length and encodes for 513 amino acids (SEQ ID NO: 10). The nucleotide sequence is 70% identical to Arabidopsis FVE and the amino acid sequence 80% identical to Arabidopsis FVE.

The GmFVE gene was cloned into a binary vector such as pBI121-Basta using restriction sites of Xba I and Sac I. The resulting construct of 35S-GmFVE was transformed into fve-b10 by Agrobacterium-mediated transformation. The presence of the transgene was confirmed by PCR analysis and selection by Basta resistance. Transgenic plants of fve-b10::35S-GmFVE were shown to have a wild-type phenotype thereby demonstrating that the GmFVE gene is functionally equivalent to the AtFVE gene.

### Example 5

### Identification of FVE Technical Features, Motifs

The conserved nature and distribution of the WD40 repeats in FVE genes provides that basis for identification of useful sequence motifs to effect FVE gene inhibition in a transgenic plant. In particular the S1, S2 and WD40 repeats have been identified as motifs useful in down-regulation of FVE gene expression and activity. One of skill in the art can analyze an FVE sequence and identify the WD40 repeat motifs using standard software, such as BLAST, CLUSTAL-W or similar programs. The S1 motif is found 5′ to the first WD40 motif and has been found only in FVE genes. The S2 motif is found between the WD40-2 and WD40-3 motifs. The S2 motif is a species specific FVE gene motif and is not strongly conserved across plant species. It still, however, provides a useful target sequence based on the unique nature of this motif and can be used in the species from which it was derived. For example, use of a corn S2 FVE sequence motif to inhibit the native corn FVE gene. WD-40 repeats have only a few conserved amino acids at spaced intervals over the 40 amino acid motif. Although WD-40 repeats are common throughout numerous genes, the sequence encoding these motifs is not highly conserved, and as such are also useful targets to down-regulate FVE activity.

### Example 6

### Constructs Useful for FVE Inhibition

According to the methods described herein, expression vector constructs can be produced using appropriate promoters and an FVE sequence of the invention. For example, any of the gene sequences of the invention may be operably linked to a promoter that is active in plant cells in such a fashion as to produce a double-strand RNA (dsRNA) molecule that results in a RNA interference (RNAi) response. Expression cassettes useful for inhibition can include the following hair pin constructs that inhibit an endogenous gene via an RNAi type mechanism. Other constructs may be generated using an antisense approach for example.

### Hairpin Constructs

For a hairpin construct, a DNA fragment is generated by producing an inverted repeat of target sequence separated by a spacer. The target sequence used can be at least 35, 50, 100, 150, 200, or 1000 nucleotides in length and the spacer should be at least 4 nucleotides in length. The sense and antisense fragments are amplified by PCR, the sense product having an XbaI site at the 5′ end and a SfiI site (5′-GGCCATCTAGGCC-3′) at the 3′ end, while the antisense product has a SacI site at the 5′ end and a SfiI site (5′-GGCCTAGATGGCC-3′) at the 3′ end. The choice of restriction site is one of convenience and other restriction sites may be used if desired. Purified sense and antisense products are both digested with SfiI, and ligated to generate fragments with a central, non-palindromic, ATCTA sequence that functions as a spacer. The resulting ligated sense-antisense fragment has a 5′ XbaI cloning site and a 3′ SacI site. The ligated fragment can be easily cloned into a transformation vector of choice. For example, a binary vector such as pBI121 using XbaI and SacI restriction sites. The constructs are confirmed by sequencing to contain the desired structure.

The following exemplary hairpin RNAi constructs were produced according the method described above:

The S1 motif (SEQ ID NO:57) was PCR amplified using the primer pair SEQ ID NOs: 177 and 178 to produce a sense fragment, and the primer pair SEQ ID NOs: 179 and 180 to produce an antisense fragment. Digestion with SfiI and ligation produced a sense-antisense hairpin cassette that was operably linked to a constitutive promoter such as a CaMV ³⁵S promoter and included a 3′ NOS terminator region (35S-HP-AtFVE-S1, SEQ ID NO:372). This construct specifically targets the FVE gene of Arabidopsis and is useful in a variety of other plant species. Similar constructs are generated using S1 sequences as described in SEQ ID NO: 51-64, 245 and 246, or identified according to the description herein.

The S2 motif (SEQ ID NO:87) was PCR amplified using the primer pair SEQ ID NOs: 189 and 190 to produce a sense fragment, and the primer pair SEQ ID NOs: 191 and 192 to produce an antisense fragment. Digestion with SfiI and ligation produced a sense-antisense hairpin cassette that was operably linked to a constitutive promoter such as a CaMV 35S promoter and included a 3′ NOS terminator region (35S-HP-AtFVE-S2, SEQ ID NO:373). This construct specifically targets the FVE gene of Arabidopsis. Similar S2-like containing constructs, specific for a species of choice, are produced using a S2 sequence as described in SEQ ID NO: 79-92, 233 and 248, or identified according to the description herein.

The S2 motif from Brassica was identified as a fragment within SEQ ID NO:206 (BnFVE) and is shown as SEQ ID NO:233. To produce the 35S-HP-BnFVE-S2 construct, a BnFVE-S2 motif was PCR amplified using the primer pair SEQ ID NOs:224 and 225 to produce a sense fragment, and the primer pair SEQ ID NOs:226 and 227 to produce an antisense fragment. Digestion with SfiI and ligation produced a sense-antisense hairpin cassette that was operably linked to a constitutive promoter such as a CaMV ³⁵S promoter and included a 3′ NOS terminator region (35S-HP-BnFVE-S2, SEQ ID NO:374).

The WD40-2 motif (SEQ ID NO: 118) was PCR amplified using the primer pair SEQ ID NOs: 185 and 186 to produce a sense fragment, and the primer pair SEQ ID NOs: 187 and 188 to produce an antisense sense fragment. Digestion with SfiI and ligation produced a sense-antisense hairpin cassette that was operably linked to a constitutive promoter such as a CaMV ³⁵S promoter and included a 3′ NOS terminator region (35S-HP-AtFVE-WD40-2, SEQ ID NO:375). This construct specifically targets the FVE gene of Arabidopsis and will be useful in a variety of other plant species based on the high degree of sequence homology. Similar WD40-2-like containing constructs are produced using a WD40-2 sequence as described in SEQ ID NO: 107-120, 234 and 252, or identified according to the description herein.

The WD40-2 motif from Brassica was identified as a fragment within SEQ ID NO:206 and is shown as SEQ ID NO:234. To produce the 35S-HP-BnFVE-WD40-2 construct a BnFVE-WD40-2 motif was PCR amplified using the primer pair SEQ ID NO's:229 and 230 to produce a sense fragment and the primer pair SEQ ID NO's:231 and 232 to produce an antisense fragment. Digestion with SfiI and ligation produced a sense-antisense hairpin cassette that was operably linked to a constitutive promoter such as a CaMV ³⁵S promoter and included a 3′ NOS terminator region (35S-HP-BnFVE-WD40-2, SEQ ID NO:376).

The WD40-1 motif (SEQ ID NO: 140) was operably linked to a constitutive promoter such as a CaMV ³⁵S promoter and included a 3′ NOS terminator region (35S-HP-AtFVE-WD40-1, SEQ ID NO:377).

The WD40-1 motif from Brassica was identified as a fragment within SEQ ID NO:206 and is shown as SEQ ID NO:235. To produce the 35S-HP-BnFVE-WD40-1 construct, a BnFVE-WD40-1 motif was PCR amplified using the primer pair SEQ ID NOs:237 and 182 to produce a sense fragment, and the primer pair SEQ ID NOs:238 and 184 to produce an antisense fragment. Digestion with SfiI and ligation produced a sense-antisense hairpin cassette that was operably linked to a constitutive promoter such as a CaMV ³⁵S promoter and included a 3′ NOS terminator region (35S-HP-BnFVE-WD40-1, SEQ ID NO:378).

The WD40-3 motif (SEQ ID NO: 174) was operably linked to a constitutive promoter such as a CaMV ³⁵S promoter and included a 3′ NOS terminator region (35S-HP-AtFVE-WD40-3, SEQ ID NO:379). Constructs are generated having a WD40-3 motif as described in SEQ ID NO: 163-176, 256 and 254.

Similarly, constructs can be generated using any of the WD40-4, WD40-5, WD40-6, motifs as described in SEQ ID NOs:273-288, 305-320 or 337-352. Additionally, constructs need not be limited to an identified domain. Constructs may be generated that incorporate sequences across motifs, provided that the sequence selected will target the endogenous FVE gene activity.

To down-regulate a target gene by a dominant-negative methodology, a non-functional version of the target gene is expressed. The gene isolated from the fve-b10 mutant described above contains a deletion that alters the encoded protein. The mutant allele is described as SEQ ID NO: 1, its predicted protein product as SEQ ID NO:2, and the genomic sequence is described as SEQ ID NO:5. The mutant coding sequence was PCR amplified using primers such as SEQ ID NO: 197 and SEQ ID NO: 198 to produce a XbaI-BamHI fragment that was cloned into pBI121ΔGUS. The fve-b10 coding sequence has an addition of 57 nucleotides of coding region and therefore makes an FVE mutant protein (fve) longer than wild-type FVE protein by 19 amino acids in the first WD40 domain, consequently destroying the first WD40 domain in fve. The fve-b10 encodes a protein with 526 amino acids in contrast to a protein of 507 amino acids in wild-type FVE. Expression of the fve-b10 gene produces an aberrant polypeptide, resulting in FVE activity inhibition. A plant having phenotypes such as increased biomass is produced. The construct (35S-fve-b10) was transformed into wild-type Arabidopsis Col-0 by Agrobacterium-mediated transformation approach. Transgenic plants of 35S-fve-b10 displayed the same phenotypes as the fve-b10 mutant plants demonstrating that the over expression of a non-functional FVE gene is sufficient to produce the desired phenotypes.

Constructs for amiRNA Down-Regulation

Artificial-miRNA down-regulation of an FVE gene is achieved by preparing a modified naturally occurring miRNA to target the desired gene sequence. A micro-RNA sequence will, when transcribed, form a stem and loop structure. The miRNA sequence comprises a first sequence that is target specific and will form one half of the stem structure with a second sequence that is essentially identical, or nearly identical, to the first but in an anti-parallel orientation relative to the first sequence, the first and second sequences separated by a non-target sequence that forms a loop structure.

Construction of amiRNA cassettes to down-regulate FVE involves a first step of PCR amplification and isolation of a miRNA171 precursor (miRNA171pre) sequence from Arabidopsis. A genomic fragment of 227 nucleotides that comprised the miRNA171pre of 123 bp was amplified from wild-type Arabidopsis accession Col-0 using primer pair of SEQ ID NOs:364 AND 365. The PCR amplified sequence is disclosed as SEQ ID NO:363.

Genomic fragment harboring miRNA171pre in
Arabidopsis (227 bp)

(SEQ ID NO:363)

TGTTTCCTTTGATATCCGCACTTTAAGCATGAGAGAGTCCCTTTGATATT
GGCCTGGTTCACTCAGATCTTACCTGACCACACACGTAGATATACATTAT
TCTCTCTAGATTATCTGATTGAGCCGCGCCAATATCTCAGTACTCTCTCG
TCTCTATTTTGGACTTTGTGGTCTTGTAGATCGATTTGTATGTGTGTGTT
GAAATGGAGACAAGTACTTGTAACTTC

Using the miRNA171pre (SEQ ID NO: 363) as a template, FVE-S1 specific primers were designed that included a 5′restriction sites to facilitate cloning, a 21 nucleotide FVE gene targeting sequence, a portion of the miR171 pre stem sequence and a partial loop sequence. In this case, the primer pair of SEQ ID NOs: 366 and 367 was used to amplify an FVE-S1 sequence. The resulting fragment (SEQ ID NO: 368) was cloned into a vector under the regulatory control of a ³⁵S promoter using the BamHI and SacI sites designed into the primers. The construct is referred to as ³⁵S-miR-At-BnFVE-S1 and is transformed into Arabidopsis and Brassica for down-regulation of FVE gene activity.

amiR171-FVE-S1-pre

(SEQ ID NO:368)

GGATCCATGAGAGAGTCCCTTTCACGTTCTCGATTTGTGCAGAGATCTTA
CCTGACCACACACGTAGATATACATTATTCTCTCTAGATTATCTCTTCAC
AAATGGAGAACGTGTCAGTACTCTCTCGTGAGCTC

To generate a construct targeting a WD40-2 motif of an FVE gene the above methodology was followed. Using the miRNA171pre (SEQ ID NO: 368) as a template, FVE-WD40-2 specific primers were designed that included a 5′restriction sites to facilitate cloning, a 21 nucleotide FVE gene targeting sequence, a portion of the miR171 pre stem sequence and a partial loop sequence. In this case, the primer pair of SEQ ID NOs: 369 and 370 was used to amplify an FVE-WD40-2 sequence. The resulting fragment (SEQ ID NO: 371) was cloned into a vector under the regulatory control of a ³⁵S promoter using the BamHI and SacI sites designed into the primers. The construct is referred to as 35S-miR-At-Bn-WD2-pre and is transformed into Arabidopsis and Brassica for down-regulation of FVE gene activity.

amiR171-FVE-WD2-pre

(SEQ ID NO:371)

GGATCCATGAGAGAGTCCCTTTACAAGTCAGATGTTTTGGGGAGATCTTA
CCTGACCACACACGTAGATATACATTATTCTCTCTAGATTATCTCCACAA
AACAACTGACTTGTTCAGTACTCTCTCGTGAGCTC

### Antisense Down Regulation

Antisense constructs are generated using an FVE sequence or portion thereof A portion is at least 50 nucleotides in length and can be 500, 1000, 1500 or more nucleotides in length. An antisense sequence is at least 70% homologous to the endogenous target sequence. More preferable the antisense sequence is from the target species and is therefore 99% or more homologous.

TABLE 6
Primers for hair pin constructs for FVE inhibition

| | | |
|---|---|---|
| | | SEQ |
| | | ID |
| Motif | DNA sequences 5′-3′ | NO: |

| | | | |
|---|---|---|---|
| BS1SF | S1 | TTT CTA GAG ATG GAA GTG TGC CCA ATA CTT | 177 |
| BS1SR | S1 | TTG GCC AAG ATG GCC TGG AGA ACG TGC TTC TTC ATT G | 178 |
| BS1AF | S1 | TTG AGC TCG ATG GAA GTG TGC CCA ATA CTT | 179 |
| BS1AR | S1 | TTG GCC ATC TAG GCC TGG AGA ACG TGC TTC TTC ATT G | 180 |
| BD1SF | WD40-1 | TTT CTA GAT TTG TGA AGA AGT ACA AGA CCA | 181 |
| BD1SR | WD40-1 | TTG GCC AAG ATG GCC ATC CCA AAT GAG AAC ATC AGG A | 182 |
| BD1AF | WD40-1 | TTG AGC TCT TTG TGA AGA AGT ACA AGA CCA | 183 |
| BD1AR | WD40-1 | TTG GCC ATC TAG GCC ATC CCA AAT GAG AAC ATC AGG A | 184 |
| BD2SF | WD40-2 | TTT CTA GAT CCC GTC CAG ATT TGA TAC TAA | 185 |
| BD2SR | WD40-2 | TTG GCC AAG ATG GCC ACT CCA CAA AAC AAC TGA CTT G | 186 |
| BD2AF | WD40-2 | TTG AGC TCT CCC GTC CAG ATT TGA TAC TAA | 187 |
| BD2AR | WD40-2 | TTG GCC ATC TAG GCC ACT CCA CAA AAC AAC TGA CTT G | 188 |
| BS2SF | S2 | TTT CTA GAA TCC AAG ATC ACA TCA CAA CGA | 189 |
| BS2SR | S2 | TTG GCC AAG ATG GCC AGT AGG ACT CTC ATT CTT ATC A | 190 |
| BS2AF | S2 | TTG AGC TCA TCC AAG ATC ACA TCA CAA CGA | 191 |
| BS2AR | S2 | TTG GCC ATC TAG GCC AGT AGG ACT CTC ATT CTT ATC A | 192 |
| BD3SF | WD40-3 | TTT CTA GAG TTG GCC CAC GAG GTG TAT ATC | 193 |
| BD3SR | WD40-3 | TTG GCC AAG ATG GCC ATC CCA TAG TAT AAG GCA AGA A | 194 |
| BD3AF | WD40-3 | TTG AGC TCG TTG GCC CAC GAG GTG TAT ATC | 195 |
| BD3AR | WD40-3 | TTG GCC ATC TAG GCC ATC CCA TAG TAT AAG GCA AGA A | 196 |
| | | For cloning into pBI121/XbaI/SacI | |

TABLE 7

Primers for expression fve-b10 dominant-
negative inhibition

| | |
|---|---|
| | SEQ |
| | ID |
| DNA sequences 5′-3′ | NO: |

| | | |
|---|---|---|
| C1F/XbaI | TTTCTAGAATGGAGAGCG ACGAAGCAGC AG | 197 |
| C1R/BamHI | TTGGATCC TTAAGGCTTGGAGGCACAAGTC | 198 |
| | For cloning into pBI121ΔGUS | |

TABLE 8
Primers for constructs for FVE inhibition in Canola

| | | SEQ |
|---|---|---|
| | | ID |
| Name | DNA sequences 5′-3′ | NO: |
| Bn-WD1-sense-Forward | TTTCTAGATTTGTGAAGAAGTTCAAGACCA | 237 |
| Bn-WD1-sense-Reverse | The same as BD1SR | 182 |
| Bn-WD1-antisense-Forward | TTGAGCTCTTTGTGAAGAAGTTCAAGACCA | 238 |
| Bn-WD1-antisense-Reverse | The same as BD1AR | 184 |
| Bn-WD2-sense-Forward | TTTCTAGATCCCGTCCTGATTTGGTATTAA | 229 |
| Bn-WD2-sense-Reverse | TTGGCCAAGATGGCCACTCCACAAAACAACTGACTTG | 230 |
| Bn-WD2-antisense-Forward | TTGAGCTCTCCCGTCCTGATTTGGTATTAA | 231 |
| Bn-WD2-antisense-Reverse | TTGGCCATCTAGGCCACTCCACAAAACAACTGACTTG | 232 |
| Bn-S2-sense-Forward | TTTCTAGAATCCAGGACCACATCACAACCG | 224 |
| Bn-S2-sense-Reverse | TTGGCCAAGATGGCCAGAAGGACTCTCAGTCTTATCA | 225 |
| Bn-S2-antisense-Forward | TTGAGCTCATCCAGGACCACATCACAACCG | 226 |
| Bn-S2-antisense-Reverse | TTGGCCATCTAGGCCAGAAGGACTCTCAGTCTTATCA | 227 |

TABLE 9

Primers for constructs for complementation test
and functional genomics

| | | SEQ |
|---|---|---|
| | | ID |
| Name | DNA sequences 5′-3′ | NO: |
| AtFVE-F | TTTCTAGAATGGAGAGCG ACGAAGCAGCAG | 197 |
| AtFVE-R | TTGGATCCTTAAGGCTTGGAGGCACAAGTC | 198 |
| Can-HF1 | TTTCTAGAATGGAGAGCGACGGAGCGGCAG | 204 |
| Can-HR2 | TTGAGCTCTCAAGGCTTGGAGGCACATGTC | 205 |
| Maize1-F | TTTCTAGAATGAAAGAGAGAGGCGGCTCCAG | 208 |
| Maize1-R | TTGAGCTCTCAATTCTTCGGTGCACAGCTGG | 209 |
| Maize2-F | TTTCTAGAATGAAGGAGAGAGGCGGCTCCAG | 210 |
| Maize2-R | TTGAGCTCTCAATTCCTCGGAGCGCAACTGG | 211 |
| PVF1 | TTTCTAGAATGAAGGAGAGGAGCGGCTCGAG | 214 |
| PVR1 | TTGAGCTCTCAGTTCCTCGGAGTGCAGCTG | 215 |
| GmF1 | TTTCTAGAATGGAGACTCCTCCTCCCCAAC | 218 |
| GmR1 | TTGAGCTCTCATTTTTCAGTCTTTGAAGCAC | 219 |
| Bn-F1 | GACCCTCAGAGAAACGGAAAATG | 201 |
| At-S3R | CATTGAACTGAGAAATGTGCTC | 202 |
| At-S2F | AGTGCACAAGAATTCTGCAGTG | 203 |
| CRLR1 | ATATGTTCAGCAGCTGCAACC | 212 |
| CRLF4 | GCTGCTGTTCTTTGTGTTCAG | 213 |

TABLE 10

Primers for construct of FVE protein
heterologous expression

| | | SEQ |
|---|---|---|
| | | ID |
| Name | DNA sequences 5′-3′ | NO: |
| At-P1F | TTGGATCCATGGAGAGCGACGAAGCAGCAG | 199 |
| At-P1R | TTGTCGACTTAAGGCTTGGAGGCACAAGTC | 200 |

TABLE 11
Primers for constructs of amiRNA

| | | SEQ |
|---|---|---|
| | | ID |
| Name | DNA sequences 5′-3′ | NO: |
| MR171F1 | TGTTTCCTTTGATATCCGCAC | 364 |
| MR171R4 | GAAGTTACAAGTACTTGTCTCC | 365 |
| MI171B-S1F | CGGGATCCATGAGAGAGTCCCTTTCACGTTCTCGAT | 366 |
| | TTGTGCAGAGATCTTACCTGACCACACACG | |
| MI171B-S1R | GCGAGCTCACGAGAGAGTACTGACACGTTCTCCATT | 367 |
| | TGTGAAGAGATAATCTAGAGAGAATAATG | |
| MI171B-D2F | CGGGATCCATGAGAGAGTCCCTTTACAAGTCAGATG | 369 |
| | TTTTGGGGAGATCTTACCTGACCACACACG | |
| MI171B-D2R | GCGAGCTCACGAGAGAGTACTGAACAAGTCAGTTGT | 370 |
| | TTTGTGGAGATAATCTAGAGAGAATAATG | |

TABLE 12

| | Primers used for FVE construct generation |
|---|---|
| Construct | SEQ ID NO's: |
| 35S-HP-AtFVE-S1 | SEQ ID NO: 177, 178, 179, 180 |
| 35S-HP-AtFVE-S2 | SEQ ID NO: 189, 190, 191, 192 |
| 35S-HP-AtFVE-WD40-1 | SEQ ID NO: 181, 182, 183, 184 |
| 35S-HP-AtFVE-WD40-2 | SEQ ID NO: 185, 186, 187, 188 |
| 35S-HP-AtFVE-WD40-3 | SEQ ID NO: 193, 194, 195, 196 |
| 35S-HP-BnFVE-S2 | SEQ ID NO: 224, 225, 226, 227 |
| 35S-HP-BnFVE-WD1 | SEQ ID NO: 237, 182, 238, 184 |
| 35S-HP-BnFVE-WD2 | SEQ ID NO: 229, 230, 231, 232 |
| 35S-AtFVE | SEQ ID NO: 197, 198 |
| 35S-fve-b10 | SEQ ID NO: 197, 198 |
| 35S-BnFVE | SEQ ID NO: 204, 205 |
| 35S-ZmFVE1 | SEQ ID NO: 208, 209 |
| 35S-ZmFVE2 | SEQ ID NO: 210, 211 |
| 35S-PvFVE | SEQ ID NO: 214, 215 |
| 35S-GmFVE | SEQ ID NO: 218, 219 |
| Ptac-AtFVE | SEQ ID NO: 199, 200 |
| Ptac-fve-b10 | SEQ ID NO: 199, 200 |
| 35S-miR-At-Bn-S1 | SEQ ID NO: 366, 367 |
| 35S-miR-At-Bn-WD2 | SEQ ID NO: 369, 370 |

### Example 7

### Plant Transformation

Arabidopsis transgenic plants were produced by the method of dipping flowering plants into an Agrobacterium culture, based on the method of Andrew Bent in, Clough S J and Bent A F, “Floral dipping: a simplified method for Agrobacterium-mediated transformation of Arabidopsis thaliana” (1998). Wild-type plants were grown under standard conditions with 16, 8 hour light dark cycles, until the plant has both developing flowers and open flowers. The plants were inverted for 2 minutes into a solution of Agrobacterium culture carrying the appropriate gene construct. Plants were then left horizontal in a tray and kept covered for two days to maintain humidity and then righted and bagged to continue growth and seed development. Mature seed was bulk harvested.

T1 plants were germinated and grown on MS plates containing kanamycin (50 μg/ml), and kanamycin resistant T1 seedlings were selected and transferred to soil for further growth. Leaf samples were harvested, DNA isolated and PCR analysis done to select those individuals containing both the desired constructs. Plants were bagged to ensure self fertilization and the T2 seed of each plant harvested separately.

Transgenic Brassica napus, Glycine max and Zea maize plants can be produced using Agrobacterium mediated transformation of cotyledon petiole tissue. Seeds are sterilized as follows. Seeds are wetted with 95% ethanol for a short period of time such as 15 seconds. Approximately 30 ml of sterilizing solution I is added (70% Javex, 100 μl Tween20) and left for approximately 15 minutes. Solution I is removed and replaced with 30 ml of solution II (0.25% mecuric chloride, 100 μl Tween-20) and incubated for about 10 minutes. Seeds are rinsed with at least 500 ml double distilled sterile water and stored in a sterile dish. Seeds are germinated on plates of ½ MS medium, pH 5.8, supplemented with 1% sucrose and 0.7% agar. Fully expanded cotyledons are harvested and placed on Medium I (Murashige minimal organics (MMO), 3% sucrose, 4.5 mg/L benzyl adenine (BA), 0.7% phytoagar, pH5.8). An Agrobacterium culture containing the nucleic acid construct of interest is grown for 2 days in AB Minimal media. The cotyledon explants are dipped such that only the cut portion of the petiole is contacted by the Agrobacterium solution. The explants are then embedded in Medium I and maintained for 5 days at 24° C., with 16, 8 hr light dark cycles. Explants are transferred to Medium II (Medium 1,300 mg/L timentin,) for a further 7 days and then to Medium III (Medium II, 20 mg/L kanamycin). Any root or shoot tissue which has developed at this time is dissected away. Explants are transferred to fresh plates of Medium III after 14-21 days. When regenerated shoot tissue develops the regenerated tissue is transferred to Medium IV (MMO, 3% sucrose, 1.0% phytoagar, 300 mg/L timentin, 20 mg/L 20 mg/L kanamycin). Once healthy shoot tissue develops, shoot tissue dissected from any callus tissue are dipped in 10× IBA and transferred to Medium V (Murashige and Skooge (MS), 3% sucrose, 0.2 mg/L indole butyric acid (IBA), 0.7% agar, 300 mg/L timentin, 20 mg/L 20 mg/L kanamycin) for rooting. Healthy plantlets are transferred to soil. The above method, with or without modifications, is suitable for the transformation of numerous plant species including Brassica napus, Glycine max, Zea maize and cotton.

Transgenic Glycine max, Zea maize and cotton can be produced using Agrobacterium-based methods which are known to one of skill in the art. Alternatively one can use a particle or non-particle biolistic bombardment transformation method. An example of non-particle biolistic transformation is given in U.S. Patent Application 20010026941, which is herein incorporated by reference in its entirety.

Agrobacterium can be used to transform variety of plant tissues according to published and established methods. Additionally, other means and methods are available to produce transgenic plants such as biolistics methods, aerosol beam injection methods, microinjection methods, in vivo site directed mutagenesis, using directed oligonucleotides for example.

### Example 8

### Physiological Evaluation of Transgenic Plants having Down-Regulated FVE Expression

Plants transformed with expression vectors designed to down-regulate FVE expression were evaluated and their physiological characteristics assessed. The vectors used included a 35S-fve-b10 construct that over expresses the mutant fve gene and affects down-regulation via a dominant-negative mechanism; a 35S-HP-AtFVE-S2 construct and a 35S-HP-AtFVE-WD40-3 construct which down-regulate FVE activity or expression via a hairpin-RNAi mechanism.

Evaluation of each set of transgenic plants comprised 7 to 10 transgenic lines plus 1 to 2 nulls, a WT control and fve-b10 mutant plants (nulls being control plants that have been isolated as negative segregants during the process of isolating homozygous transgenic lines). Eight replicate plants were planted per entry and per harvest. Two destructive harvests were set up, first harvest at 5 days post first flower and second harvest at maturity. During plant growth and before the two destructive harvests non-destructive measurements were made at: the vegetative stage of 12 day old seedlings when the number of leaves were counted; and at the time of first open flower when the number of leaves and plant height were recorded. At the time of the two harvests, the number of leaves, plant height and number of branches were also counted in addition to total shoot biomass determinations.

The results of these growth studies showed that all three down-regulation constructs targeting the FVE gene successfully caused an enhancement in biomass production in the transgenic plants and delay in flowering. The differences between transgenic and control plants were first apparent shortly before flowering time.

### 35S-fve-b10 Dominant-Negative Construct

The results often 35S-fve-b10 transgenic lines are shown in Table 13, and demonstrate delayed flowering of 2 to 5 days as compared to WT and null controls, whereas fve-b10 mutant plants in this experiment flowered 15 days later than WT. Transgenic lines of this construct had up to 60% more leaves at 5 days from first open flower and their dry weight was increased up to 2.6 fold over the WT. The stem DW was enhanced up to 58% and the total shoot biomass enhancement was up to 2 fold over the WT. The fve-b10 mutant showed almost 4 fold increase in shoot biomass.

Two of the transgenic lines (lines 57-6 and 73-8) that have shown a delay in flowering and enhanced shoot biomass plus the WT control and the fve-b10 mutant were examined for main stem live cross sections 3 days post-first flower. These were examined using a dissection scope (Lica) and photos were taken at 8× magnification. From the photo measurement of the stem diameter, the thickness of the intervesicular fiber layer and counts of vascular bundles were determined. The two transgenic lines had 42 and 65% thicker stems (compared to 60% thicker in fve-b10) than those of WT. The transgenic lines had 48 and 56% more vascular bundles (compared to 87% in fve-b10), and 33% and 51% thicker fiber layer (compared to 47% in fve-b10). In addition, live epidermal peels were obtained from the stems of these plants and were examined using fluorescent microscope for chloroplast numbers in guard cells. The fve-b10 plants had 20% more chloroplasts while the transgenic plants had 10 to 15% more chloroplasts than WT controls.

TABLE 13
Growth enhancement in 35S-fve-b10 transgenic lines
(5 days post-flower)

| | | | | | | Shoot |
|---|---|---|---|---|---|---|
| | Days to | Stem DW | Rosette | Leaf DW | Shoot | DW (% |
| entry | flower | (mg) | leaf # | (mg) | DW (mg) | of WT) |
| fve- | 36 | 360 ± 42 | 75 | 991 ± 70 | 1351 ± 104 | 384 |
| b10 | | | | | | |
| 73-8 | 26 | 289 ± 22 | 46 | 437 ± 47 | 727 ± 63 | 207 |
| 95-4 | 25 | 287 ± 28 | 50 | 400 ± 30 | 686 ± 51 | 195 |
| 98-2 | 26 | 275 ± 18 | 50 | 365 ± 21 | 641 ± 28 | 182 |
| 62-2 | 26 | 207 ± 12 | 44 | 415 ± 52 | 622 ± 59 | 177 |
| 57-6 | 25 | 258 ± 18 | 42 | 360 ± 31 | 619 ± 46 | 176 |
| 71-3 | 25 | 232 ± 19 | 43 | 347 ± 24 | 579 ± 38 | 165 |
| 70-6 | 24 | 215 ± 10 | 42 | 351 ± 18 | 567 ± 26 | 161 |
| 81-7 | 25 | 223 ± 13 | 42 | 340 ± 27 | 563 ± 36 | 160 |
| 68-2 | 23 | 225 ± 15 | 40 | 259 ± 12 | 484 ± 24 | 138 |
| 71-4 | 20 | 153 ± 13 | 31 | 153 ± 6 | 310 ± 1 | 87 |
| (null) | | | | | | |
| 98-4 | 21 | 160 ± 13 | 33 | 179 ± 14 | 340 ± 3 | 97 |
| (null) | | | | | | |
| WT | 21 | 183 ± 13 | 32 | 168 ± 11 | 350 ± 2 | 100 |

A group of plants (n=8) grown in 4″ pots was grown to maturity and plants were harvested at the end of their life cycle for final shoot biomass and seed yield. The harvest index was also calculated. The results showed that the final total shoot biomass of the transgenic lines was also enhanced from 5% to 32% over that of controls. The seed yield varied from WT levels to 13% greater than that of controls. As a result the harvest index was reduced in all of the transgenic lines indicating, as shown before for the fve-b10 mutant, that transgenic plants direct more of their resources towards the biomass than the seed production.

### 35S-fve-b10 Dominant-Negative Construct In Brassica

### Physiological Characterization of Transgenic Canola Plants.

Brassica plants were transformed with a 35S-fve-b10 construct for dominant-negative inhibition of the Brassica FVE gene. Eleven transgenic events at a T1 stage were assessed for biomass enhancement and time to flowering. At a T1 stage the plants were confirmed as transgenic by PCR analysis, however the plants were heterozygous. These transgenic plants were grown in a greenhouse in 4″ pots under optimal conditions (22° C., 18 hr light at 400uE). Days to first open flower were recorded and nine days from first flower all plants were harvested for leaf and stem dry biomass determinations. Plant height, number of leaves and number of branches were also recorded. The results indicate that three of the transgenic events had enhanced shoot biomass and delayed flowering (Table 14). The delay was up to seven days in the best event (165G-6) and the shoot biomass enhancement was increased as much as 40% relative to WT. There were no differences in plant height, number of branches and leaf number. The enhancement in the shoot biomass seemed to be a result of thicker stems, as indicated by greater stem DW, and either larger or thicker leaves as indicated by greater leaf DW. The transgenic events were heterozygous and this contributes to increased variability in the data. The plants were known to be transgenic however the degree of FVE inhibition was undetermined. Homozygous plants are selected and physiological analysis and best lines are selected.

The construct used to transform canola was an Arabidopsis construct (35S-fve-b10) further demonstrating the universality of the method and the cross species portability of the gene.

TABLE 14
Growth enhancement in 35S-fve-b10 transgenic lines

| | | | | | | Shoot |
|---|---|---|---|---|---|---|
| | Days to | Stem | | Leaf DW | Shoot | DW (% |
| entry | flower | DW (g) | leaf # | (g) | DW (g) | of WT) |
| 161G-1 | 41 | 8.9 ± 0.5 | 30 | 5.3 ± 0.9 | 14.2 ± 1.3 | 114% |
| 165G-4 | 42 | 9.5 ± 0.9 | 28 | 5.0 ± 0.6 | 14.5 ± 1.4 | 117% |
| 165G-6 | 45 | 10.2 ± 1.0 | 30 | 7.2 ± 1.6 | 17.4 ± 2.5 | 140% |
| 159Y-5 | 39 | 6.7 ± 0.4 | 28 | 4.1 ± 0.2 | 10.8 ± 0.5 | 87% |
| (null) | | | | | | |
| WT | 38 | 8.3 ± 0.5 | 28 | 4.1 ± 0.3 | 12.4 ± 0.7 | 100% |

### 35S-HP-AtFVE-S2 Construct in Arabidopsis

The results of the experiment with eight 35S-HP-AtFVE-S2 transgenic lines as compared to WT and null controls is shown in Table 15. Transgenic plants showed a delay in flowering of up to 6 days as compared to WT, while fve-b10 mutant in this study was delayed 13 days. Five days into flowering, transgenic lines showed up to twice as many rosette leaves, up to 2.4 fold greater stem biomass and up to 3.4 fold greater leaf biomass. The total shoot dry weight (DW) was up to 2.5 fold greater in the transgenic lines while the fve-b10 mutant showed 5.4 fold enhancement of its shoot biomass.

TABLE 15
Growth enhancement in 35S-HP-AtFVE-S2 transgenic lines

| | | | Ro- | | | Shoot |
|---|---|---|---|---|---|---|
| | Days to | Stem DW | sette | Leaf DW | Shoot | DW (% |
| entry | flower | (g) | leaf # | (g) | DW (g) | of WT) |
| fve- | 35 | 255 ± 24 | 79 | 974 ± 36 | 1212 ± 41 | 539 |
| b10 | | | | | | |
| 30-10 | 28 | 257 ± 22 | 33 | 311 ± 25 | 568 ± 39 | 253 |
| 48-1 | 27 | 167 ± 17 | 52 | 395 ± 42 | 562 ± 55 | 250 |
| 36-9 | 26 | 187 ± 17 | 50 | 336 ± 28 | 523 ± 39 | 233 |
| 28-9 | 25 | 224 ± 25 | 40 | 252 ± 20 | 477 ± 43 | 212 |
| 40-4 | 26 | 226 ± 16 | 38 | 238 ± 14 | 465 ± 20 | 207 |
| 49-2 | 24 | 179 ± 13 | 34 | 215 ± 12 | 394 ± 22 | 175 |
| 6-3 | 24 | 175 ± 19 | 40 | 219 ± 32 | 394 ± 50 | 175 |
| 14-9 | 25 | 193 ± 21 | 32 | 182 ± 16 | 375 ± 35 | 167 |
| 14-4 | 22 | 114 ± 12 | 28 | 136 ± 11 | 249 ± 23 | 111 |
| (null) | | | | | | |
| WT | 22 | 108 ± 14 | 26 | 117 ± 10 | 225 ± 22 | 100 |

A group of plants (n=8) was also grown to maturity and final total dry biomass and seed yield of the plants were determined at the end of their lifecycle. The results showed a range in biomass enhancement of up to 45% greater than the controls. The seed yield was increased in transgenic lines up to 30% over the controls and the harvest index was reduced accordingly.

### 35S-HP-AtFVE-WD40-3 Construct

The results of the experiment with five 35S-HP-AtFVE-WD40-3 transgenic lines (Table 16) showed a delay in flowering by as much as 10 days as compared to WT, while fve-b10 mutant in this study was delayed by 13 days. During the mid-flower harvest (5 days from first flower) transgenic lines showed up to 2.5 fold more rosette leaves, 1.6 fold greater stem dry weight and 4.4 fold greater leaf dry weight. The total shoot dry biomass was up to 3 fold greater in transgenics while the fve-b10 mutant showed 3.9 fold enhancement.

TABLE 16
Growth enhancement in 35S-HP-AtFVE-WD40-3 transgenic

| | | | Ro- | | | Shoot |
|---|---|---|---|---|---|---|
| | Days to | Stem DW | sette | Leaf DW | Shoot | DW (% |
| entry | flower | (g) | leaf # | (g) | DW (g) | of WT) |
| fve- | 35 | 324 ± 36 | 103 | 1266 ± 69 | 1609 ± 91 | 388 |
| b10 | | | | | | |
| 57-4 | 33 | 321 ± 25 | 84 | 936 ± 77 | 1256 ± 99 | 303 |
| 10-7 | 27 | 241 ± 25 | 55 | 543 ± 48 | 785 ± 66 | 189 |
| 55-3 | 27 | 297 ± 32 | 51 | 467 ± 45 | 765 ± 73 | 184 |
| 30-8 | 26 | 255 ± 16 | 45 | 403 ± 31 | 658 ± 38 | 159 |
| 5-10 | 24 | 233 ± 22 | 34 | 228 ± 10 | 462 ± 28 | 111 |
| 10-1 | 22 | 154 ± 12 | 29 | 166 ± 7 | 320 ± 19 | 77 |
| (null) | | | | | | |
| WT | 23 | 202 ± 12 | 33 | 212 ± 8 | 415 ± 18 | 100 |

In conclusion, all three constructs aimed at down-regulation of the FVE gene resulted in transgenic lines which showed biomass enhancement and a delay in flowering phenotypes similar to those of the fve-b10 mutant. The magnitude of the phenotypes observed spans a range between mutant and wild-type. This is expected as transgenic plants produce a range of gene inhibition in each line based on factors such as, insertion site, copy number and strength of transgene expression for example. Such a range of responses is typical and one uses such a varied response to select the lines having the most desirable phenotypes. In addition to enhanced shoot biomass down-regulation of FVE also results in increased chloroplast numbers and therefore higher chlorophyll content, thicker stems and enhanced vascular bundles. More vascular bundles and thicker intervesicular fiber layer results in higher fiber content of the stems.

### Example 9

### Analysis of Cellular Structure in Stems of Arabidopsis fve-b10 and Transgenic Plants having Inhibited FVE Activity

A series of microscopic studies using various tissue types during plant development were analyzed for structural differences between FVE inhibited plants including the mutant fve-b10 and wild-type control plants. Leaf, root and stem tissues were collected at four developmental stages: young seedling (7 days after germination), bolting, first day of flowering, and post-flowering (fourth day of flowering). Tissues were fixed using paraformaldehyde, dehydrated using a graded ethanol series, and infiltrated using paraffin/TBA method and embedded. Blocks were sectioned, and sections were mounted on slides. Slides were stained either with Toluidine Blue O to visualize the cross-sections of the tissues, or with Calcofluor White to illustrate any qualitative change in cellulose content in these sections.

### Toluidine Blue O Staining

Sections were deparaffinized in two changes of CitriSolv (Thermo Fisher Scientific), rinsed in absolute ethanol and allowed to dry. A filtered 1% stock solution of Toluidine Blue O (TBO) was prepared in 1% sodium borate. Stock solution was diluted 10-fold in sodium borate before use (final concentration—0.1% TBO in 1% sodium borate). Sections were stained for 30-60 seconds and rinsed thoroughly in deionized water. Slides were allowed to dry, and cover slips were mounted using Permount (Thermo Fisher Scientific). Sections were observed and photographed using a Leica DMLB microscope with a Leica DFC300FX camera.

### Cellulose Staining

Calcofluor White (Fluorescent Brightener 28, Sigma-Aldrich) was used as a 0.1% solution in half-normal saline pH 6.0. Sections were deparaffinized in 2 changes of CitriSolv, and re-hydrated using a graded ethanol series. Slides were washed under running tap water for 48 hours to remove all traces of organic solvents. After washing Calcoflour solution was applied to the sections and a cover slip placed over the sections. (Harrington and Raper, 1968) Sections were observed and photographed using a Leica DMLB microscope equipped with a BFP-A-Basic filter cube (Semrock—excitation filter BP340-380, dichromatic mirror 400 and suppression filter LP425) and Leica DFC300FX camera.

Data demonstrated that stems of fve-b10 show approximately 100% increase in the number of vascular bundles, increased cellulose content and have more cells with unchanged cell size therefore the stems are physically larger in diameter compared to the control at each developmental stage investigated. The vascular tissue regions in leaves and roots of fve-b10 are also enlarged while individual cell size remains unchanged resulting in the leaves and roots being thicker, and cellulose density is higher than controls.

### Example 10

### Enhanced Fiber Content in the Stems of fve-b10 and Transgenic Plants

Quantitative analysis of fiber content (both cellulose and lignin) in plants having reduced FVE activity was performed. Stem tissues were collected from the Arabidopsis fve-b10 mutant, transgenic plants expressing the mutant gene construct 35S-fve-b10 and thereby inhibiting native FVE activity via a dominant negative mechanism and their respective controls (Col-0 and a segregated null). Plant tissues were collected on the seventh day of flowering for cellulose and lignin analysis. The total cellulose content including cellulose and hemi-cellulose in the stem tissues was measured by sugar monomers derived from complete hydrolysis of cellulose and hemi-cellulose using high performance liquid chromatography (HPLC). The sugar monomers quantified included fructose, arabinose, rhamnose, galactose, glucose, xylose and mannose. Glucose is the major breakdown product of cellulose, and xylose presents in the largest amount in hemi-cellulose (Rogers et al., 2005). Values were expressed as a percent of its appropriate wild-type control or a segregated null. In parallel, the total lignin content including acid-insoluble and acid-soluble in these stem tissues was obtained by using Klason method (Chiang and Funaoka, 1990), and TAPPI Useful Method UM250 (TAPPI useful method UM250, 1991), respectively.

The mutant fve-b10 and the dominant negative transgenic line demonstrated 21% and 15% increase in total sugar content respectively and in turn estimated 137% and 118% higher sugar content per plant compared to their appropriate controls. Similarly, the mutant fve-b10 and the dominant negative transgenic line contained respective 17% and 8% higher total lignin, and in turn estimated 131% and 104% more total lignin per plant compared to their appropriate controls. Although acid-insoluble and acid-soluble lignin content/plant was approximately increased by 197% and 43% for fve-b10, and by 139% and 31% for the transgenic line, respectively, only acid-insoluble lignin content was increased by 51% and 27% for fve-b10 and the transgenic line, respectively. In contrast, acid-soluble lignin content was reduced by 27% and 30% for fve-b10 and the transgenic line, respectively (all figures shown are statically significant at 5%). These results are summarized in Tables 17 and 18.

In conclusion, inhibition of FVE activity in Arabidopsis increases total cellulose and lignin content in the stems, also alters the composition of cellulose and lignin, and even results in a slight change in proportion of cellulose and lignin in favor of cellulose content. Since the sequence of FVE is very conserved evolutionarily among various plant species, the effect of inhibition of FVE in Arabidopsis ought to be highly conserved in other plant species.

The fve-b10 mutant and the 35S-fve-b10 transgenic plants both had greater levels of each sugar, albeit to different degrees.

TABLE 17
Fiber analysis of fve-b10 and transgenic plants relative to WT

| | Total | | Acid Insoluble | Acid Soluble |
|---|---|---|---|---|
| Entry | Sugars | Total Lignin | Lignin | Lignin |
| fve-b10 | 121% | 117% | 151% | 73% |
| 35S-fve-b10 | 115% | 108% | 127% | 70% |
| WT Columbia | 100% | 100% | 100% | 100% |

TABLE 18
Sugar monomer analysis expressed as % of control

| Entry | fucose | arabinose | rhamnose | galactose | glucose | xylose | mannose |
|---|---|---|---|---|---|---|---|
| fve-b10 | 438 | 204 | 105 | 160 | 114 | 131 | 108 |
| 35S-fve-b10 | 114 | 149 | 109 | 116 | 111 | 127 | 111 |

### Example 11

### Western Blot Analysis of Plants

Western blot analysis was performed on transgenic Arabidopsis lines 35S-HP-FVE-S2, 35S-HP-FVE-WD40-3 and 35S-FVE in the fve-b10 mutant. Over-expression of FVE in Arabidopsis mutant fve-b10 restores wild-type protein levels and reverts an fve-b10 phenotype to essentially that of a wild-type plant. Inhibition of FVE expression in Arabidopsis by either ³⁵S—HP-FVE-S2 or 35S-HP-FVE-WD40-3 reduces or eliminates detectable FVE protein.

The antibody produced by against Arabidopsis FVE can recognize maize-FVE expressed in transformed Arabidopsis A fve-b10 plants with the ³⁵S-ZmFVE construct. The maize-FVE expression levels are also correlated with biomass production in the same manner of AtFVE, which was verified with the maize FVE functional genomics analysis.

### Example 12

### Amino Acid and Nucleic Acid Sequence Analysis of FVE and Motif Identification

Below is a comparison of FVE motif amino acid sequences and nucleotide sequences from selected plant species. Sequence homology and multiple sequence alignments were performed using ClustalW at http://www.ebi.ac.uk/clustalw/.

### Exemplar S1 Motifs

| | | | |
|---|---|---|---|
| MAIZE-2 | DGSVPNTLVIANCEVVKPRVAAAEHISQFNEEARSP | 36 | SEQ ID NO:37 |
| SORGHUM | DGSVPNTLVIANCEVVKPRVAAAEHISQFNEEARSP | 36 | SEQ ID NO:38 |
| MAIZE-1 | DGSVPNTLVIANCEVVKPRVAAAEHISQFNEEARSP | 36 | SEQ ID NO:39 |
| SOYBEAN-1 | DGSVPNTLVIANCEVVKPRVAAAEHISQFNEEARSP | 36 | SEQ ID NO:40 |
| ARABIDOPSIS | DGSVPNTLVIANCEVVKPRVAAAEHISQFNEEARSP | 36 | SEQ ID NO:41 |
| PISUM-SATIVUM | DGSVPNTLVIANCEVVKTRVAAAEHISQFNEEARSP | 36 | SEQ ID NO:42 |
| MEDICAGO-1 | DGSVPNTLVIANCEVVKTRVAAAEHISQFNEEARSP | 36 | SEQ ID NO:43 |
| POPLAR | DGSVPNTLVIANCDVVKSRVAAAEHISQFNEEARSP | 36 | SEQ ID NO:44 |
| COTTON | DGSVPNTLVIANCEVVKPRVAAAEHISQFNEKTRSP | 36 | SEQ ID NO:45 |
| PETUNIA | DGSVPNTLVIANCEVVKPRVAAAEHISQFNEESRSP | 36 | SEQ ID NO:46 |
| RICE-1 | DGTVPNTLVIANCEVVKPRVAAAEHISQFNEEARSP | 36 | SEQ ID NO:47 |
| RICE-2 | DGTVPNTLVIANCEVVKPRVAAAEHISQFNEEARSP | 36 | SEQ ID NO:48 |
| GRAPE | DGSVPNTLVIANCEVVKPRVAAAEHIAQFNEEARSP | 36 | SEQ ID NO:49 |
| MEDICAGO-2 | DGTAPNTLVIATCEIVKPRVAAAEHIAMFNEEARSP | 36 | SEQ ID NO:50 |
| CANOLA | DGSVPNTLVIANCEVVKPRVAAAEHISQFNEEARSP | 36 | SEQ ID NO:243 |
| SWITCHGRASS | DGSVPNTLVIANCEVVKPRVAAAEHISQFNEEARSP | 36 | SEQ ID NO:244 |
| PISUM-SATIVUM | GATGGTAGTGTCCCAAATACTTTGGTGATTGCGAATTGTGAGGTTGTGAAAACTAGGGTT | | |
| MEDICAGO-1 | GATGGAAGTGTTCCGAATACTTTGGTGATTGCGAATTGTGAGGTTGTGAAGACTAGGGTT | | |
| SOYBEAN-1 | GATGGTAGTGTGCCGAATACTCTGGTGATTGCGAATTGCGAGGTTGTGAAGCCTAGGGTT | | |
| COTTON | GATGGTAGTGTTCCGAATACTCTTGTGATTGCTAATTGTGAAGTTGTCAAACCTAGGGTT | | |
| POPLAR | GATGGTAGTGTTCCAAATACTTTGGTCATTGCAAATTGTGATGTTGTCAAGTCTAGAGTT | | |
| PETUNIA | GATGGAAGTGTGCCAAACACTTTGGTTATAGCAAATTGTGAAGTTGTTAAACCTAGGGTT | | |
| ARABIDOPSIS | GATGGAAGTGTGCCCAATACTTTGGTCATAGCAAATTGTGAAGTTGTTAAGCCAAGGGTT | | |
| GRAPE | GATGGCAGTGTTCCGAATACCTTAGTTATAGCAAACTGTGAAGTTGTTAAACCTAGGGTA | | |
| MAIZE-2 | GATGGGAGTGTGCCTAATACTCTGGTTATCGCAAACTGTGAAGTTGTGAAACCAAGGGTT | | |
| SORGHUM | GATGGGAGTGTGCCTAATACTCTAGTTATCGCAAACTGTGAAGTTGTGAAGCCACGGGTT | | |
| MAIZE-1 | GATGGGAGTGTGCCTAATACTCTGGTTATCGCAAATTGTGAAGTTGTGAAACCTAGGGTT | | |
| RICE-1 | GATGGGACTGTCCCTAATACTCTGGTTATTGCAAATTGTGAAGTTGTCAAACCAAGGGTT | | |
| RICE-2 | GATGGGACTGTCCCTAATACTCTGGTTATTGCAAATTGTGAAGTTGTCAAACCAAGGGTT | | |
| MEDICAGO-2 | GATGGAACTGCTCCCAATACGCTTGTTATTGCTACTTGTGAAATTGTTAAACCTAGGGTT | | |
| CANOLA | GATGGCAGTGTGCCCAACACTCTAGTCATAGCGAATTGCGAAGTTGTTAAGCCAAGGGTC | | |
| SWITCHGRASS | GATGGGAGTGTGCCTAATACTCTGGTTATTGCGAACTGTGAAGTTGTCAAACCAAGGGTT | | |
| PISUM-SATIVUM | GCAGCTGCAGAGCATATTTCACAGTTTAATGAGGAGGCTCGCTCTCCA | 108 | SEQ ID NO:51 |
| MEDICAGO-1 | GCAGCTGCTGAACACATTTCTCAGTTTAATGAAGAGGCTCGCTCTCCA | 108 | SEQ ID NO:52 |
| SOYBEAN-1 | GCTGCTGCTGAGCACATTTCGCAGTTTAATGAAGAGGCGCGGTCCCCA | 108 | SEQ ID NO:53 |
| COTTON | GCTGCTGCTGAGCACATATCTCAGTTTAATGAAAAAACACGCTCACCA | 108 | SEQ ID NO:54 |
| POPLAR | GCTGCTGCGGAACACATATCTCAGTTTAATGAAGAAGCACGCTCTCCA | 108 | SEQ ID NO:55 |
| PETUNIA | GCAGCTGCAGAGCACATATCACAGTTCAATGAAGAATCACGATCTCCT | 108 | SEQ ID NO:56 |
| ARABIDOPSIS | GCTGCAGCAGAGCACATTTCTCAGTTCAATGAAGAAGCACGTTCTCCA | 108 | SEQ ID NO:57 |
| GRAPE | GCTGCCGCAGAGCATATTGCTCAGTTCAACGAGGAAGCGCGGTCACCC | 108 | SEQ ID NO:58 |
| MAIZE-2 | GCAGCTGCTGAACATATCTCGCAGTTTAACGAGGAAGCACGATCACCT | 108 | SEQ ID NO:59 |
| SORGHUM | GCAGCTGCTGAACATATCTCGCAGTTTAACGAGGAAGCACGATCACCT | 108 | SEQ ID NO:60 |
| MAIZE-1 | GCAGCTGCTGAACATATCTCACAGTTCAATGAGGAAGCACGGTCACCT | 108 | SEQ ID NO:61 |
| RICE-1 | GCAGCTGCTGAACATATATCTCAGTTCAATGAGGAAGCACGATCACCT | 108 | SEQ ID NO:62 |
| RICE-2 | GCAGCTGCTGAACATATATCTCAGTTCAATGAGGAAGCACGATCACCT | 108 | SEQ ID NO:63 |
| MEDICAGO-2 | GCTGCTGCTGAACATATTGCTATGTTTAATGAAGAGGCGCGTTCTCCA | 108 | SEQ ID NO:64 |
| CANOLA | GCTGCAGCAGAGCACATATCTCAGTTCAACGAAGAAGCACGTTCTCCA | 108 | SEQ ID NO:245 |
| SWITCHGRASS | GCAGCTGCTGAACATATCTCACAGTTCAATGAGGAAGCACGATCGCCT | 108 | SEQ ID NO:246 |

Exemplar S2 motifs

| | | | |
|---|---|---|---|
| COTTON | IQDHITTMATDP-TKSPGSGGSIIKQN-KPGEGNDKAADGPS | 40 | SEQ ID NO:65 |
| POPLAR | IQDHITSSASDPATKSPGSGGSIIK---KTGDGSDKATDGPS | 39 | SEQ ID NO:66 |
| SOYBEAN-1 | IEDHITSAATDS-----KSGGSIIKQNSKSGEGNDKTADGPT | 37 | SEQ ID NO:67 |
| PISUM-SATIVUM | IEDHVTSAATD------KSGGSIIKPNSKSGEGNDKTVDSPS | 36 | SEQ ID NO:68 |
| MEDICAGO-1 | IEDHITSAATDS----NKSGGSIAK-----------TADSPT | 27 | SEQ ID NO:69 |
| MEDICAGO-2 | IHDHIATLATE-------EEPDVNE-GSNVGGNSEKAAQSPS | 34 | SEQ ID NO:70 |
| GRAPE | IQDHISTLAADP-----GSAKST----SKAGGGNDKPVESPS | 33 | SEQ ID NO:71 |
| PETUNIA | IQDHISTLSTDA-----QKPAGFIK----PATTSIKAGDNPS | 33 | SEQ ID NO:72 |
| ARABIDOPSIS | IQDHITTIG-TD----SKSSGSIIK---QTGEGTD-KNESPT | 33 | SEQ ID NO:73 |
| MAIZE-1 | IQDHISALGDSS-----SSPGASGS---KQSIKTANEKESPK | 34 | SEQ ID NO:74 |
| MAIZE-2 | IQDHISALGDSS-----SSPGASGS---KQSGKIANEKESPK | 34 | SEQ ID NO:75 |
| SORGHUM | IQDHISALGDSS-----SSPGASGS---KQSGKSATEKESPK | 34 | SEQ ID NO:76 |
| RICE-1 | IQDHISALGDSSKT--ESSPGASGS---K--GKTANDKDSPK | 35 | SEQ ID NO:77 |
| RICE-2 | IQDHISALGDSSKT--ESSPGASGS---K--GKTANDKDSPK | 35 | SEQ ID NO:78 |
| CANOLA | IQDHITTAGSTD----SKSSGSIIK---QTGEGGD-KTESPS | 34 | SEQ ID NO:223 |
| SWITCHGRASS | IQDHISALGDSS-----SSPGASGS---KQSGKTANEKESPK | 34 | SEQ ID NO:247 |
| MEDICAGO-2 | -ATTCATGATCATATTGCAACTTTAGCTA------CAGA------------AGAAGAACC | 41 | |
| GRAPE | -ATTCAGGATCACATCTCGACTTTGGCAG------CAGA------------TCCAGGGTC | 41 | |
| PISUM-ATIVUM | -ATTGAAGACCATGTAACATCTGCTGCTA------CGGAC------AAGTCTGGTGGATC | 47 | |
| COTTON | -ATCCAGGACCACATAACAACAATGGCTA------CAGACCCTACTAAATCTCCTGGATC | 53 | |
| POPLAR | -ATCCAGGACCATATAACATCATCTGCCTCTGATCCAG---CAACTAAGTCTCCAGGATC | 56 | |
| SOYBEAN-1 | -ATTGAAGACCATATAACATCTGCTGCCA------CAGACTCC---AAATCTGGTGGGTC | 50 | |
| MEDICAGO-1 | -ATTGAAGACCATATAACATCTGCTGCTA------CAGACTCCAACAAGTCTGGTGGATC | 53 | |
| PETUNIA | -ATTCAAGATCATATATCAACATTGAGCA------CAGATGCACAAAAACCTGCTGGTTT | 53 | |
| ARABIDOPSIS | -ATCCAAGATCACATCACAACGATTGGGA------CAGATTCCA---AATCATCTGGATC | 50 | |
| MAIZE-2 | -ATCCAAGACCACATATCTGCCCTTGGGGATTCCTCG---------TCTTCTCCCGGAGC | 50 | |
| SORGHUM | -ATCCAAGACCACATATCTGCCCTTGGGGATTCCTCG---------TCTTCTCCTGGAGC | 50 | |
| MAIZE-1 | -ATCCAAGACCATATATCTGCCCTTGGGGATTCCTCG---------TCTTCTCCTGGAGC | 50 | |
| RICE-1 | -ATCCAAGACCACATATCTGCACTGGGAGATTCCTCGAAAACTGAATCTTCTCCAGGGGC | 59 | |
| RICE-2 | -ATCCAAGACCACATATCTGCACTGGGAGATTCCTCGAAAACTGAATCTTCTCCAGGGGC | 59 | |
| CANOLA | -ATCCAGGACCACATCACAACCGCTGGTAGTA---CAGACTCCA---AATCATCTGGATC | 53 | |
| SWITCHGRASS | -ATCCAAGACCACATATCTGCCCTTGGGGATTCCTCA---------TCTTCTCCTGGAGC | 50 | |
| MEDICAGO-2 | C---------GATGTTAATGAGGGCTCTAATGTTGGGGGAAATAGCGAGAAAGCTGCACA | 92 | |
| GRAPE | ------------AGCAAAGAGTACCTCTAAGGCTGGTGGTGGTAATGATAAACCTGTAGA | 89 | |
| PISUM-SATIVUM | C---------ATTATCAAACCGAACTCTAAATCTGGGGAAGGCAATGACAAAACTGTTGA | 98 | |
| COTTON | TGGCGGATCAATCATCAAACAAAAC---AAGCCTGGGGAAGGTAATGACAAAGCTGCTGA | 110 | |
| POPLAR | TGGTGGATCAATCATTAAAAAGA---------CTGGGGATGGCAGTGATAAAGCCACTGA | 107 | |
| SOYBEAN-1 | A---------ATTATCAAACAAAACTCTAAATCTGGAGAAGGCAATGATAAAACTGCTGA | 101 | |
| MEDICAGO-1 | C---------ATTGCCAAAACTGCTGATAGCCCTACT----------------------- | 81 | |
| PETUNIA | C------------ATCAAGCCAG---------CCACTACATCTATT---AAGGCTGGTGA | 89 | |
| ARABIDOPSIS | T---------ATCATCAAACAGA---------CTGGTGAAGGTACTGATAAGAATG---A | 89 | |
| MAIZE-2 | A------TCTGGCAGCAAGCAGTCTGGCAAAATTGCAAATGAAAAGGAGAGTCCTAAA-- | 102 | |
| SORGHUM | A------TCTGGCAGCAAGCAGTCTGGTAAATCTGCAACTGAAAAGGAGAGCCCTAAA-- | 102 | |
| MAIZE-1 | A------TCTGGCAGCAAGCAGTCTATTAAAACTGCAAATGAAAAGGAGAGCCCTAAA-- | 102 | |
| RICE-1 | A------TCAGGAAGCAAG------GGAAAAACTGCAAATGATAAGGATAGTCCTAAA-- | 105 | |
| RICE-2 | A------TCAGGAAGCAAG------GGAAAAACTGCAAATGATAAGGATAGTCCTAAA-- | 105 | |
| CANOLA | C---------ATCATCAAACAGA---------CTGGTGAAGGTGGTGATAAGACTG---A | 92 | |
| SWITCHGRASS | A------TCTGGCAGCAAGCAGTCTGGCAAAACTGCGAATGAAAAGGAGAGTCCCAAA-- | 102 | |
| MEDICAGO-2 | AAGCCCGTCT | 102 | SEQ ID NO:79 |
| GRAPE | AAGCCCTTCT | 99 | SEQ ID NO:80 |
| PISUM-SATIVUM | TAGCCCTTCT | 108 | SEQ ID NO:81 |
| COTTON | TGGGCCTTCT | 120 | SEQ ID NO:82 |
| POPLAR | TGGCCCTTCT | 117 | SEQ ID NO:83 |
| SOYBEAN-1 | TGGCCCTACT | 111 | SEQ ID NO:84 |
| MEDICAGO-1 | ---------- | | SEQ ID NO:85 |
| PETUNIA | TAATCCCTCT | 99 | SEQ ID NO:86 |
| ARABIDOPSIS | GAGTCCTACT | 99 | SEQ ID NO:87 |
| MAIZE-2 | ---------- | | SEQ ID NO:88 |
| SORGHUM | ---------- | | SEQ ID NO:89 |
| MAIZE-1 | ---------- | | SEQ ID NO:90 |
| RICE-1 | ---------- | | SEQ ID NO:91 |
| RICE-2 | ---------- | | SEQ ID NO:92 |
| CANOLA | GAGTCCTTCT | 102 | SEQ ID NO:233 |
| SWITCHGRASS | ---------- | | SEQ ID NO:248 |

### Exemplar WD40-1 Motifs

| | | | |
|---|---|---|---|
| POPLAR | FVKKYKTIIHPGEVNRIRELPQNSKIVATHTDSPDVLIWD | 40 | SEQ ID NO:121 |
| ARABIDOPSIS | FVKKYKTIIHPGEVNRIRELPQNSKIVATHTDSPDVLIWD | 40 | SEQ ID NO:122 |
| PISUM-SATIVUM | FVKKYKTIIHPGEVNRIRELPQNSKIVATHTDSPDVLIWD | 40 | SEQ ID NO:123 |
| MEDICAGO-1 | FVKKYKTIIHPGEVNRIRELPQNSKIVATHTDSPDVLIWD | 40 | SEQ ID NO:124 |
| MAIZE-1 | FVKKYKTIVHPGEVNRIRELPQNSKIIATHTDSPDVLIWD | 40 | SEQ ID NO:125 |
| SORGHUM | FVKKYKTIVHPGEVNRIRELPQNSKIIATHTDSPDVLIWD | 40 | SEQ ID NO:126 |
| MAIZE-2 | FVKKYKTIVHPGEVNRIRELPQNSKIIATHTDSPDVLVWD | 40 | SEQ ID NO:127 |
| RICE-1 | FVKKYKTIIHPGEVNRIRELPQNSKIIATHTDSPDVLIWD | 40 | SEQ ID NO:128 |
| RICE-2 | FVKKYKTIIHPGEVNRIRELPQNSKIIATHTDSPDVLIWD | 40 | SEQ ID NO:129 |
| SOYBEAN-1 | FVKKYKTIIHPGEVNRIRELPQNSKIVATHTDSPDVLVWD | 40 | SEQ ID NO:130 |
| GRAPE | FVKKFKTIIHPGEVNRIRELPQNSKIVATHTDSPDVLIWD | 40 | SEQ ID NO:131 |
| COTTON | FVKKYKTIIHPGEVNRIKELPQSSRIVATHTDSPDVLIWD | 40 | SEQ ID NO:132 |
| PETUNIA | FVKKYKTIIHPGEVNRIRELPQNKNIVATHTDSPEVLIWD | 40 | SEQ ID NO:133 |
| MEDICAGO-2 | FVKKVKTILHPGEVNRIRELPSNTNIVATHTDSPNVMIWN | 40 | SEQ ID NO:134 |
| CANOLA | FVKKFKTIIHPGEVNRIRELPQNSKIIATHTDSPDVLIWD | 40 | SEQ ID NO:236 |
| SWITCHGRASS | FVKKYKTIVHPGEVNRIRELPQNSKIIATHTDSPDVLIWD | 40 | SEQ ID NO:249 |
| PISUM-SATIVUM | TTTGTTAAGAAGTACAAGACCATCATACATCCCGGGGAGGTGAACAGAATTAGGGAATTG | 60 | |
| MEDICAGO-1 | TTTGTCAAGAAGTACAAGACTATCATACATCCAGGCGAGGTGAACAGAATTAGGGAATTG | 60 | |
| SOYBEAN-1 | TTTGTGAAGAAGTACAAGACCATCATACATCCTGGTGAGGTAAACAGAATTAGGGAATTG | 60 | |
| POPLAR | TTTGTTAAGAAGTACAAGACCATCATACATCCTGGAGAGGTAAACAGAATCAGAGAACTC | 60 | |
| COTTON | TTTGTGAAGAAGTACAAAACCATCATACATCCTGGAGAAGTCAACAGAATCAAGGAACTT | 60 | |
| ARABIDOPSIS | TTTGTGAAGAAGTACAAGACCATCATTCACCCTGGAGAGGTTAACAGAATCAGGGAACTC | 60 | |
| MAIZE-2 | TTTGTAAAGAAGTACAAGACTATAGTTCATCCTGGTGAAGTTAACAGAATCAGGGAGCTT | 60 | |
| SORGHUM | TTTGTTAAGAAGTACAAGACCATAGTTCATCCTGGTGAAGTTAACAGAATCAGGGAGCTT | 60 | |
| MAIZE-1 | TTTGTAAAGAAGTACAAAACTATAGTTCATCCTGGTGAGGTTAACAGAATCAGGGAACTT | 60 | |
| RICE-1 | TTTGTAAAGAAGTACAAGACTATAATTCATCCAGGCGAGGTGAACAGGATTAGGGAGCTT | 60 | |
| RICE-2 | TTTGTAAAGAAGTACAAGACTATAATTCATCCAGGCGAGGTGAACAGGATTAGGGAGCTT | 60 | |
| GRAPE | TTTGTAAAGAAGTTCAAAACAATTATACATCCAGGAGAGGTGAACCGAATCAGGGAACTG | 60 | |
| PETUNIA | TTTGTCAAGAAGTACAAAACCATTATACACCCAGGGGAGGTTAACAGAATCAGAGAGCTT | 60 | |
| MEDICAGO-2 | TTTGTTAAGAAGGTTAAAACTATTCTTCATCCCGGTGAGGTAAATAGAATCAGAGAACTC | 60 | |
| CANOLA | TTTGTGAAGAAGTTCAAGACCATCATTCACCCTGGAGAGGTTAACCGAATCAGGGAACTC | 60 | |
| SWITCHGRASS | TTTGTGAAGAAGTATAAGACTATAGTTCATCCTGGTGAGGTTAACAGAATCAGGGAGCTT | 60 | |
| PISUM-SATIVUM | CCGCAAAATTCTAAGATAGTGGCTACTCACACAGACAGCCCTGATGTTCTCATTTGGGAT | 120 | SEQ ID NO:135 |
| MEDICAGO-1 | CCACAAAATTCTAAGATTGTGGCCACTCACACAGACAGCCCTGATGTTCTCATTTGGGAT | 120 | SEQ ID NO:136 |
| SOYBEAN-1 | CCACAAAATTCCAAGATAGTGGCTACACATACAGACAGCCCTGATGTCCTTGTTTGGGAT | 120 | SEQ ID NO:137 |
| POPLAR | CCCCAGAATAGTAAGATAGTGGCTACTCATACTGACAGCCCTGATGTTCTTATATGGGAT | 120 | SEQ ID NO:138 |
| COTTON | CCACAGAGCTCTAGGATTGTGGCAACTCACACTGATAGTCCTGATGTTCTTATTTGGGAT | 120 | SEQ ID NO:139 |
| ARABIDOPSIS | CCACAGAATAGTAAGATTGTTGCTACTCACACCGACAGTCCTGATGTTCTCATTTGGGAT | 120 | SEQ ID NO:140 |
| MAIZE-2 | CCACAGAACAGTAAGATCATAGCCACACACACTGACAGTCCAGATGTACTTGTTTGGGAT | 120 | SEQ ID NO:141 |
| SORGHUM | CCACAGAACAGTAAGATCATAGCCACTCACACTGACAGTCCAGATGTACTTATTTGGGAT | 120 | SEQ ID NO:142 |
| MAIZE-1 | CCACAGAACAGTAAGATCATAGCCACTCACACTGACAGTCCAGATGTACTTATTTGGGAT | 120 | SEQ ID NO:143 |
| RICE-1 | CCGCAGAACAGTAAGATCATTGCCACTCATACCGACAGCCCAGATGTTCTCATTTGGGAT | 120 | SEQ ID NO:144 |
| RICE-2 | CCGCAGAACAGTAAGATCATTGCCACTCATACCGACAGCCCAGATGTTCTCATTTGGGAT | 120 | SEQ ID NO:145 |
| GRAPE | CCACAGAATAGTAAGATAGTGGCCACACACACTGACAGTCCTGATGTCCTCATTTGGGAT | 120 | SEQ ID NO:146 |
| PETUNIA | CCTCAAAATAAAAACATAGTGGCAACCCATACTGATAGTCCTGAAGTTCTAATTTGGGAT | 120 | SEQ ID NO:147 |
| MEDICAGO-2 | CCGTCAAATACTAATATAGTTGCCACACATACAGATAGTCCAAATGTTATGATTTGGAAT | 120 | SEQ ID NO:148 |
| CANOLA | CCACAAAACAGTAAGATTATTGCTACTCACACCGACAGTCCTGATGTTCTCATTTGGGAT | 120 | SEQ ID NO:235 |
| SWITCHGRASS | CCACAGAACAGTAAGATCATAGCCACTCACACCGACAGTCCAGATGTACTTATTTGGGAT | 120 | SEQ ID NO:250 |

Exemplar WD40-2 motifs

| | | | |
|---|---|---|---|
| SOYBEAN-1 | SRPDLILTGHQDNAEFALAMCPTEPYVLSGGKDKTVVLWS | 40 | SEQ ID NO:93 |
| PISUM-SATIVUM | SRPDLILTGHQDNAEFALAMCPTEPYVLSGGKDKTVVLWS | 40 | SEQ ID NO:94 |
| MEDICAGO-1 | SRPDLILTGHQDNAEFALAMCPTQPYVLSGGKDKTVVLWS | 40 | SEQ ID NO:95 |
| POPLAR | SRPDLILTGHQDNAEFALAMCPTDPYVLSGGKDKFVVLWS | 40 | SEQ ID NO:96 |
| COTTON | SRPDLILTGHQDNAEFALAMCPTEPYVLSGGKDKSVVLWS | 40 | SEQ ID NO:97 |
| MAIZE-1 | SRPDLILTGHKENAEFALAMCPAEPYVLSGGKDKSVVLWS | 40 | SEQ ID NO:98 |
| SORGHUM | SRPDLILTGHKENAEFALAMCPAEPYVLSGGKDKSVVLWS | 40 | SEQ ID NO:99 |
| RICE-1 | SRPDLILRGHKDIAEFALAMCPAEPYVLSGGKDKSVVWWS | 40 | SEQ ID NO:100 |
| RICE-2 | SRPDLILRGHKDIAEFALAMCPAEPYVLSGGKDKSVVWWS | 40 | SEQ ID NO:101 |
| MAIZE-2 | SRPDLILTGHQENAEFALAMCPAEPYVLSGGKDKFVVLWS | 40 | SEQ ID NO:102 |
| ARABIDOPSIS | SRPDLILTGHQDNAEFALAMCPTEPFVLSGGKDKSVVLWS | 40 | SEQ ID NO:103 |
| PETUNIA | SRPDLTLIGHSENAEFALAMCPTEPFVLSGGKDKSVVLWS | 40 | SEQ ID NO:104 |
| GRAPE | KVLFLILTGHKDNAEFALAMCPTEPLVLSGGKDKSVVLWS | 40 | SEQ ID NO:105 |
| MEDICAGO-2 | SIPDLVLTGHKDNAEFALAMCSTEPFVLSGGRDKLVVLWS | 40 | SEQ ID NO:106 |
| CANOLA | SRPDLVLTGHQDNAEFXLAMCPTEPFVLSGGKDKSVVLWS | 40 | SEQ ID NO:228 |
| SWITCHGRASS | SRPDLILTGHQENAEFALAMCPAEPYVLSGGKDKSVVLWS | 40 | SEQ ID NO:251 |
| PISUM-SATIVUM | TCTCGTCCAGATT-TGATATTGACCGGACACCAAGACAATGCTGAGTTTGCTCTTGCGAT | 59 | |
| MEDICAGO-1 | TCTCGTCCAGATC-TGATATTGACTGGACACCAAGATAATGCAGAGTTTGCTCTTGCAAT | 59 | |
| SOYBEAN-1 | TCTCGTCCTGATT-TGATATTGACTGGACACCAAGATAATGCGGAATTTGCTCTTGCAAT | 59 | |
| COTTON | TCTCGCCCAGATT-TGATTTTGACTGGGCATCAAGATAATGCTGAATTTGCTCTTGCAAT | 59 | |
| POPLAR | TCTCGTCCAGATT-TGATTTTGACTGGACATCAAGACAATGCTGAGTTTGCCCTTGCAAT | 59 | |
| SORGHUM | TCTCGCCCTGATC-TGATATTAACGGGACATAAGGAAAATGCGGAATTTGCGCTTGCTAT | 59 | |
| MAIZE-1 | TCTCGCCCTGATC-TGATATTAACAGGACATAAGGAAAATGCGGAATTTGCGCTTGCCAT | 59 | |
| MAIZE-2 | TCTCGTCCTGATC-TGATATTAACGGGACACCAGGAAAATGCAGAATTTGCGCTTGCCAT | 59 | |
| RICE-1 | TCTCGTCCTGATT-TGATATTAAGAGGACATAAGGATATTGCTGAGTTTGCGCTTGCTAT | 59 | |
| RICE-2 | TCTCGCCCTGATT-TGATATTAAGAGGACATAAGGATATTGCTGAGTTTGCGCTTGCTAT | 59 | |
| PETUNIA | TCACGCCCAGATT-TGACATTGATTGGACATAGTGAGAATGCAGAATTTGCACTGGCAAT | 59 | |
| ARABIDOPSIS | TCCCGTCCAGATT-TGATACTAACTGGGCACCAAGATAATGCTGAATTTGCTCTTGCCAT | 59 | |
| MEDICAGO-2 | TCCATCCCAGACT-TGGTATTAACTGGACATAAGGATAATGCTGAATTTGCACTAGCTAT | 59 | |
| GRAPE | -AAGGTTTTGTTCCTGATATTAACTGGGCATAAAGATAATGCAGAATTTGCTCTTGCCAT | 59 | |
| CANOLA | TCCCGTCCTGATT-TGGTATTAACTGGACATCAAGACAATGCTGAATTCSCTCTTGCAAT | 59 | |
| SWITCHGRASS | TCTCGGCCTGATC-TGATATTAACAGGACATCAGGAAAATGCTGAATTCGCGCTTGCCAT | 59 | |
| PISUM-SATIVUM | GTGCCCAACTGAGCCTTATGTCCTTTCAGGAGGAAAAGATAAAACAGTGGTGTTGTGGAG | 119 | |
| MEDICAGO-1 | GTGCCCTACTCAGCCCTATGTGCTTTCTGGAGGAAAAGACAAAACAGTCGTGTTGTGGAG | 119 | |
| SOYBEAN-1 | GTGCCCAACTGAACCCTATGTTCTTTCAGGAGGAAAGGACAAAACAGTGGTGTTGTGGAG | 119 | |
| COTTON | GTGTCCAACTGAGCCTTATGTGCTCTCTGGAGGGAAGGACAAATCAGTGGTTTTGTGGAG | 119 | |
| POPLAR | GTGCCCAACTGATCCCTATGTGCTTTCTGGAGGGAAGGACAAGTTCGTAGTTTTGTGGAG | 119 | |
| SORGHUM | GTGCCCAGCAGAACCATATGTCCTATCAGGAGGAAAGGACAAATCTGTTGTCTTGTGGAG | 119 | |
| MAIZE-1 | GTGTCCAGCAGAACCATATGTCCTATCAGGAGGAAAGGACAAATCTGTTGTCTTGTGGAG | 119 | |
| MAIZE-2 | GTGTCCAGCAGAACCATATGTCCTGTCAGGAGGAAAGGACAAATTTGTTGTCTTGTGGAG | 119 | |
| RICE-1 | GTGCCCAGCTGAGCCATATGTGTTATCTGGAGGAAAAGACAAATCTGTTGTATGGTGGAG | 119 | |
| RICE-2 | GTGCCCAGCTGAGCCATATGTGTTATCTGGAGGAAAAGACAAATCTGTTGTATGGTGGAG | 119 | |
| PETUNIA | GTGCCCCACTGAACCCTTTGTGCTCTCTGGAGGAAAGGACAAATCTGTGGTACTGTGGAG | 119 | |
| ARABIDOPSIS | GTGCCCAACGGAACCCTTTGTGCTCTCCGGAGGCAAGGACAAGTCAGTTGTTTTGTGGAG | 119 | |
| MEDICAGO-2 | GTGTTCAACTGAGCCCTTTGTTCTTTCCGGAGGGAGAGACAAGCTTGTGGTGTTATGGAG | 119 | |
| GRAPE | GTGTCCAACTGAACCATTGGTGCTCTCTGGAGGCAAGGATAAGTCTGTGGTGTTGTGGAG | 119 | |
| CANOLA | GTGCCCAACCGAACCCTTTGTCCTCTCTGGAGGCAAAGACAAGTCAGTTGTTTTGTGGAG | 119 | |
| SWITCHGRASS | GTGTCCAGCAGAACCATATGTACTGTCAGGAGGAAAGGACAAATCTGTTGTCTTGTGGAG | 119 | |
| PISUM-SATIVUM | T | 120 | SEQ ID NO:107 |
| MEDICAGO-1 | T | 120 | SEQ ID NO:108 |
| SOYBEAN-1 | T | 120 | SEQ ID NO:109 |
| COTTON | C | 120 | SEQ ID NO:110 |
| POPLAR | T | 120 | SEQ ID NO:111 |
| SORGHUM | C | 120 | SEQ ID NO:112 |
| MAIZE-1 | C | 120 | SEQ ID NO:113 |
| MAIZE-2 | C | 120 | SEQ ID NO:114 |
| RICE-1 | C | 120 | SEQ ID NO:115 |
| RICE-2 | C | 120 | SEQ ID NO:116 |
| PETUNIA | T | 120 | SEQ ID NO:117 |
| ARABIDOPSIS | T | 120 | SEQ ID NO:118 |
| MEDICAGO-2 | T | 120 | SEQ ID NO:119 |
| GRAPE | T | 120 | SEQ ID NO:120 |
| CANOLA | T | 120 | SEQ ID NO:234 |
| SWITCHGRASS | C | 120 | SEQ ID NO:252 |

### Exemplar WD40-3 D Motifs

| | | | |
|---|---|---|---|
| GRAPE | IGARGIYQGHDDTVEDVQFCPLSAQEFCSVGDDSCLILWD | 40 | SEQ ID NO:149 |
| PETUNIA | IQARGIFQGHEDTVEDVQFCPSSSQEFCSVGDDSCLILWD | 40 | SEQ ID NO:150 |
| PISUM-SATIVUM | VGPRGIYSGHDDTVEDVAFCPSSAQEFCSVGDDSCLILWD | 40 | SEQ ID NO:151 |
| MEDICAGO-1 | VGPRGIYSGHEDTVEDVAFCPSSAQEFCSVGDDSCLILWD | 40 | SEQ ID NO:152 |
| POPLAR | VGPRGIYQGHEDTVEDVAFCPSSAQEFCSVGDDSCLILWD | 40 | SEQ ID NO:153 |
| ARABIDOPSIS | VGPRGVYHGHEDTVEDVAFSPTSAQEFCSVGDDSCLILWD | 40 | SEQ ID NO:154 |
| COTTON | LGPRGVFCGHEDTVEDVTFCPSSAQEFCSVGDDSCLILWD | 40 | SEQ ID NO:155 |
| SOYBEAN-1 | VGPRGIYCGHEDTVEDVTFCPSSAQEFCSVGDDSCLILWD | 40 | SEQ ID NO:156 |
| MEDICAGO-2 | VGARGVYRGHKDTVEDVQFCPSSAQEFCSVGDDSCLILWD | 40 | SEQ ID NO:157 |
| MAIZE-1 | VDPRGIFHGHDSTVEDVQFCPSSAQEFCSVGDDACLILWD | 40 | SEQ ID NO:158 |
| MAIZE-2 | VDPRGIFHGHDSTVEDVQFCPSSAQEFCSVGDDACLILWD | 40 | SEQ ID NO:159 |
| RICE-1 | VDPRGIFLGHDSTVEDVQFCPSSAQEFCSVGDDSCLILWD | 40 | SEQ ID NO:160 |
| RICE-2 | VDPRGIFLGHDSTVEDVQFCPSSAQEFCSVGDDSCLILWD | 40 | SEQ ID NO:161 |
| SORGHUM | VDPRGIFCG------------------------------- | 9 | SEQ ID NO:162 |
| CANOLA | LGPRGVYHGHDDTVEDVAFSPTSAQEFCSVGDDSCLILWD | 40 | SEQ ID NO:255 |
| SWITCHGRASS | VDPRGIFHGHDSTVEDVQFCPSSAQEFCSVGDDACLILWD | 40 | SEQ ID NO:253 |
| PISUM-SATIVUM | GTCGGGCCAAGAGGTATCTACTCTGGGCACGATGATACTGTTGAAGATGTGGCCTTTTGC | 60 | |
| MEDICAGO-1 | GTCGGACCAAGAGGTATCTACTCTGGGCATGAGGATACTGTTGAAGACGTGGCTTTTTGC | 60 | |
| SOYBEAN-1 | GTTGGACCACGAGGCATTTATTGTGGGCATGAGGATACAGTTGAAGATGTGACTTTCTGC | 60 | |
| MAIZE-2 | GTTGATCCTAGAGGTATATTCCATGGCCATGACAGTACTGTTGAAGATGTTCAGTTCTGC | 60 | |
| MAIZE-1 | GTTGATCCTAGAGGTATATTTCATGGACATGACAGCACGGTTGAAGATGTTCAGTTCTGC | 60 | |
| RICE-1 | GTTGATCCTCGTGGTATCTTTCTTGGCCACGACAGTACTGTTGAAGATGTCCAGTTCTGC | 60 | |
| RICE-2 | GTTGATCCTCGTGGTATCTTTCTTGGCCACGACAGTACTGTTGAAGATGTCCAGTTCTGC | 60 | |
| PETUNIA | ATTCAAGCTCGTGGAATCTTCCAAGGCCATGAGGATACCGTTGAAGATGTTCAGTTCTGC | 60 | |
| COTTON | TTAGGTCCTCGTGGTGTCTTCTGTGGGCATGAGGATACAGTTGAAGATGTTACATTCTGT | 60 | |
| POPLAR | GTTGGACCGCGAGGTATCTACCAAGGGCATGAGGATACAGTTGAAGACGTGGCATTCTGT | 60 | |
| GRAPE | ATAGGAGCACGTGGTATCTACCAAGGGCATGATGATACTGTTGAAGATGTGCAATTCTGC | 60 | |
| ARABIDOPSIS | GTTGGCCCACGAGGTGTATATCATGGCCATGAAGATACAGTTGAAGATGTGGCATTCAGC | 60 | |
| MEDICAGO-2 | GTTGGAGCAAGGGGCGTCTACCGGGGTCATAAAGACACTGTTGAAGATGTGCAGTTTTGC | 60 | |
| SORGHUM | GTTGATCCTAGAGGTATATTCTGTGGG--------------------------------- | 27 | |
| CANOLA | CTTGGCCCGCGAGGTGTATATCATGGCCATGATGATACCGTTGAAGATGTCGCTTTCAGC | 60 | |
| SWITCHGRASS | GTTGATCCTCGGGGTATATTCCATGGACATGACAGCACTGTTGAAGATGTTCAGTTCTGC | 60 | |
| PISUM-SATIVUM | CCTTCTAGTGCGCAGGAATTCTGTAGTGTTGGAGATGATTCTTGTCTCATATTATGGGAT | 120 | SEQ ID NO:163 |
| MEDICAGO-1 | CCTTCTAGTGCGCAGGAGTTCTGTAGTGTTGGAGATGATTCTTGTCTCATATTATGGGAT | 120 | SEQ ID NO:164 |
| SOYBEAN-1 | CCATCTAGTGCACAGGAGTTCTGTAGTGTTGGAGATGATTCTTGTCTCATCTTATGGGAT | 120 | SEQ ID NO:165 |
| MAIZE-2 | CCTTCCAGTGCGCAGGAGTTTTGTAGTGTGGGTGATGATGCTTGTCTTATTCTGTGGGAT | 120 | SEQ ID NO:166 |
| MAIZE-1 | CCTTCCAGTGCGCAGGAGTTTTGTAGTGTGGGTGATGATGCTTGTCTTATTCTGTGGGAT | 120 | SEQ ID NO:167 |
| RICE-1 | CCTTCTAGTGCACAGGAGTTTTGTAGTGTAGGCGATGATTCTTGTCTTATTCTTTGGGAT | 120 | SEQ ID NO:168 |
| RICE-2 | CCTTCTAGTGCACAGGAGTTTTGTAGTGTGGGCGATGATTCTTGTCTTATTCTTTGGGAT | 120 | SEQ ID NO:169 |
| PETUNIA | CCATCAAGTTCACAGGAATTCTGTAGTGTTGGTGATGATTCATGTCTCATTCTATGGGAT | 120 | SEQ ID NO:170 |
| COTTON | CCATCAAGTGCACAAGAGTTTTGTAGTGTAGGTGATGATTCCTGCCTCATACTATGGGAT | 120 | SEQ ID NO:171 |
| POPLAR | CCATCCAGTGCGCAGGAGTTTTGTAGTGTAGGAGATGATTCTTGCCTTATATTATGGGAT | 120 | SEQ ID NO:172 |
| GRAPE | CCATTAAGCGCACAGGAGTTCTGTAGCGTAGGTGATGATTCTTGCCTTATCTTATGGGAT | 120 | SEQ ID NO:173 |
| ARABIDOPSIS | CCGACGAGTGCACAAGAATTCTGCAGTGTTGGTGATGATTCTTGCCTTATACTATGGGAT | 120 | SEQ ID NO:174 |
| MEDICAGO-2 | CCCTCAAGTGCACAGGAGTTCTGTAGTGTAGGTGATGATTCTTGTCTCATACTCTGGGAT | 120 | SEQ ID NO:175 |
| SORGHUM | ------------------------------------------------------------ | | SEQ ID NO:176 |
| CANOLA | CCCACTAGTGCACAAGAGTTCTGCAGTGTCGGTGACGATTCTTGCCTTATACTATGGGAT | 120 | SEQ ID NO:256 |
| SWITCHGRASS | CCTTCCAGTGCACAGGAATTCTGTAGTGTGGGTGATGATGCTTGTCTTATTCTCTGGGAT | 120 | SEQ ID NO:254 |

### Exemplar WD40-4 D Motifs

| | | | |
|---|---|---|---|
| ARABIDOPSIS | NPVTKVEKAHDADLHCVDWNPHDDNLILTGSADNTVRLFD | 40 | SEQ ID NO:257 |
| PISUM-SATIVUM | SPVVKVEKAHNADLHCVDWNPHDDNLILTGSADNSVRLFD | 40 | SEQ ID NO:258 |
| PETUNIA | SPVVKVEKAHNADLHCVDWNPHDGNFIITGSADNSVRLFD | 40 | SEQ ID NO:259 |
| SOYBEAN-1 | SPVVKVEKAHNADLHCVDWNPHDDNLILTGSADNSVRMFD | 40 | SEQ ID NO:260 |
| MEDICAGO-1 | SPVVKVEKAHDADLHCVDWNPHDDNLILTGSADNSIRMFD | 40 | SEQ ID NO:261 |
| POPLAR | SPAIKVERAHNADLHCVDWNPQDDNLILTGSADTSVCMFD | 40 | SEQ ID NO:262 |
| COTTON | GPTVKVEKAHNADLHCVDWNPHDDNLILTGSADHTVRMFD | 40 | SEQ ID NO:263 |
| GRAPE | TPAIKVEKAHNADLHCVDWNPHDINLILTGSADNTVRMFD | 40 | SEQ ID NO:264 |
| MAIZE-1 | APAVKVEKAHSGDVHCVDWNPLDVNYILTGSADNSVRMWD | 40 | SEQ ID NO:265 |
| SORGHUM | ----------------VDWNPLDVNYILTGSADNSVRMWD | 24 | SEQ ID NO:266 |
| MAIZE-2 | DPAVKVEKAHSGDVHCVDWNPLDVNYILTGSADNSVRMWD | 40 | SEQ ID NO:267 |
| SWITCHGRASS | SPAVKVEKAHSGDVHCVDWNPLDVNYILTGSADNSVRMWD | 40 | SEQ ID NO:268 |
| RICE-1 | GPAVKVEKAHGGDVHCVDWNLHDVNYILTGSADNSVRMWD | 40 | SEQ ID NO:269 |
| RICE-2 | GPAVKVEKAHGGDVHCVDWNLHDVNYILTG---------- | 30 | SEQ ID NO:270 |
| MEDICAGO-2 | FPAVKVEKAHDGDVHCVDWNTHDINFILTGSADNTVRMFD | 40 | SEQ ID NO:271 |
| CANOLA | SPVTKVEKAHDADLHCVDWNPHDDNLILTGSADNTVRLYD | 40 | SEQ ID NO:272 |
| PISUM-SATIVUM | AGTCCTGTTGTTAAGGTTGAAAAAGCTCATAATGCCGATCTTCACTGTGTTGACTGGAAT | 60 | |
| MEDICAGO-1 | AGTCCTGTGGTTAAGGTTGAAAAAGCTCATGATGCTGATCTTCACTGTGTTGACTGGAAT | 60 | |
| SOYBEAN-1 | AGCCCTGTGGTTAAGGTTGAGAAAGCTCATAATGCTGATCTTCACTGTGTGGACTGGAAT | 60 | |
| ARABIDOPSIS | AACCCTGTCACGAAGGTTGAAAAAGCGCATGATGCTGATCTTCATTGTGTTGATTGGAAT | 60 | |
| CANOLA | AGCCCTGTCACGAAGGTTGAAAAGGCGCACGATGCTGATCTTCATTGTGTCGATTGGAAC | 60 | |
| COTTON | GGCCCCACTGTCAAGGTTGAAAAGGCACATAATGCTGATCTCCATTGTGTTGATTGGAAC | 60 | |
| POPLAR | AGCCCAGCTATAAAGGTTGAAAGGGCACATAATGCTGATCTTCACTGTGTTGATTGGAAT | 60 | |
| PETUNIA | AGTCCAGTTGTAAAGGTTGAAAAAGCTCATAATGCTGATCTCCACTGTGTTGACTGGAAT | 60 | |
| MEDICAGO-2 | TTTCCAGCTGTCAAGGTTGAGAAGGCACATGATGGAGATGTACATTGCGTTGATTGGAAT | 60 | |
| GRAPE | ACTCCAGCCATCAAGGTCGAGAAAGCTCATAATGCTGATCTTCACTGTGTTGATTGGAAT | 60 | |
| MAIZE-2 | GACCCAGCTGTTAAGGTTGAGAAAGCTCACAGTGGAGATGTTCATTGTGTTGATTGGAAT | 60 | |
| SORGHUM | ------------------------------------------------GTTGATTGGAAT | 12 | |
| MAIZE-1 | GCCCCAGCTGTTAAGGTTGAGAAAGCTCACAGTGGAGATGTTCATTGTGTTGATTGGAAT | 60 | |
| SWITCHGRASS | AGCCCGGCTGTTAAGGTTGAGAAAGCTCATAGTGGGGATGTTCATTGTGTGGATTGGAAT | 60 | |
| RICE-1 | GGCCCAGCTGTTAAGGTTGAGAAAGCTCATGGTGGTGATGTTCATTGTGTTGACTGGAAC | 60 | |
| RICE-2 | GGCCCAGCTGTTAAGGTTGAGAAAGCTCATGGTGGTGATGTTCATTGTGTTGACTGGAAC | 60 | |
| PISUM-SATIVUM | CCCCATGATGATAATCTGATTCTTACTGGGTCGGCAGATAATTCTGTTCGCTTGTTTGAT | 120 | SEQ ID NO:273 |
| MEDICAGO-1 | CCCCATGATGATAATCTGATTCTTACTGGGTCGGCAGATAATTCTATTCGCATGTTTGAT | 120 | SEQ ID NO:274 |
| SOYBEAN-1 | CCCCATGATGATAATCTGATTCTTACTGGGTCAGCAGATAATTCTGTTCGCATGTTTGAT | 120 | SEQ ID NO:275 |
| ARABIDOPSIS | CCTCATGACGACAATCTGATCCTGACAGGGTCAGCAGACAACACTGTCCGGTTGTTTGAT | 120 | SEQ ID NO:276 |
| CANOLA | CCTCATGATGACAATCTGATCCTGACAGGGTCTGCAGACAACACTGTTCGGTTGTATGAT | 120 | SEQ ID NO:277 |
| COTTON | CCCCATGATGACAATCTTATCCTGACTGGGTCTGCAGATCATACTGTACGTATGTTTGAT | 120 | SEQ ID NO:278 |
| POPLAR | CCTCAAGATGATAATCTCATTTTAACTGGGTCTGCAGATACTTCTGTTTGCATGTTTGAT | 120 | SEQ ID NO:279 |
| PETUNIA | CCTCATGATGGCAACTTTATTATCACTGGATCTGCAGATAATTCTGTCCGCTTGTTTGAT | 120 | SEQ ID NO:280 |
| MEDICAGO-2 | ACTCATGACATCAATTTTATTCTGACTGGCTCTGCTGATAACACAGTTCGCATGTTTGAT | 120 | SEQ ID NO:281 |
| GRAPE | CCTCATGACATAAATCTTATTCTAACTGGATCGGCTGACAATACTGTTCGCATGTTTGAT | 120 | SEQ ID NO:282 |
| MAIZE-2 | CCCCTTGATGTTAACTATATCTTAACTGGTTCTGCCGATAACTCTGTCCGAATGTGGGAT | 120 | SEQ ID NO:283 |
| SORGHUM | CCCCTTGACGTTAACTATATCTTAACTGGTTCTGCCGATAACTCTGTCCGAATGTGGGAT | 72 | SEQ ID NO:284 |
| MAIZE-1 | CCCCTTGATGTTAACTATATCTTAACTGGGTCTGCCGATAACTCTGTCCGAATGTGGGAT | 120 | SEQ ID NO:285 |
| SWITCHGRASS | CCGCTTGATGTTAACTATATCTTAACTGGTTCTGCTGATAACTCTGTCCGTATGTGGGAT | 120 | SEQ ID NO:286 |
| RICE-1 | CTCCATGATGTTAACTATATCTTAACTGGTTCTGCGGATAATTCTGTCCGTATGTGGGAC | 120 | SEQ ID NO:287 |
| RICE-2 | CTCCATGATGTTAACTATATCTTAACTGGT------------------------------ | 90 | SEQ ID NO:288 |

### Exemplar WD40-5 D Motifs

| | | | |
|---|---|---|---|
| SWITCHGRASS | GSPIHKFEGHKAAVLCVQWSPDPASVFGSSAEDGFLNVWD | 40 | SEQ ID NO:289 |
| MAIZE-2 | GSPIHKFEGHKAAVLCVQWSPDPASVFGSSAEDGFLNVWD | 40 | SEQ ID NO:290 |
| MAIZE-1 | GSPIHKFEGHKAAVLCVQWSPDPASVFGSSAEDGFLNVWD | 40 | SEQ ID NO:291 |
| SORGHUM | SSPIHKFEGHKAAVLCVQWSPDPASVFGSSAEDGFLNVWD | 40 | SEQ ID NO:292 |
| RICE-1 | GIPVHKFEGHKAAVLCVQWSPDKASVFGSSAEDGFLNVWD | 40 | SEQ ID NO:293 |
| RICE-2 | ------------------WSPDKASVFGSSAEDGFLNVWD | 22 | SEQ ID NO:294 |
| GRAPE | GSPIHTFEGHTAAVLCVQWSPDKASIFGSSAEDGILNLWN | 40 | SEQ ID NO:295 |
| COTTON | GSPIYKFEGHKAAVLCVQWSPDKSSVFGSSAEDGLLNIWD | 40 | SEQ ID NO:296 |
| ARABIDOPSIS | GSPIYKFEGHKAAVLCVQWSPDKSSVFGSSAEDGLLNIWD | 40 | SEQ ID NO:297 |
| CANOLA | GTPIYKFEGHKAAVLCVQWSPDKSSVFGSSAEDGLLNIWD | 40 | SEQ ID NO:298 |
| SOYBEAN-1 | GSPIHKFEGHKAAVLCVQWSPDKSSVFGSSAEDGLLNIWD | 40 | SEQ ID NO:299 |
| PISUM-SATIVUM | GSPIHKFEAHKAAVLCVQWSPDKSSVFGSSAEDGLLNIWD | 40 | SEQ ID NO:300 |
| MEDICAGO-1 | GSPIHKFEAHKAAVLCVQWSPDKSSVFGSSAEDGLLNIWD | 40 | SEQ ID NO:301 |
| POPLAR | GLPVYKFEGHKAAVLCVQWSPDKASVFGSSAEDGLLNIWD | 40 | SEQ ID NO:302 |
| PETUNIA | GSPVHIFENHKAAVLCVQWCPDRSSVFGSTAEDGRLNIWD | 40 | SEQ ID NO:303 |
| MEDICAGO-2 | GSPVYKFEGHDEPVLCVQWNPAKSSVFGSGAEDGIINIWD | 40 | SEQ ID NO:304 |
| RICE-1 | GGCATTCCAGTTCACAAATTTGAGGGTCATAAAGCTGCTGTTCTTTGTGTTCAGTGGTCA | 60 | |
| RICE-2 | ------------------------------------------------------TGGTCA | 6 | |
| SORGHUM | AGTTCTCCAATTCATAAATTTGAGGGCCATAAAGCTGCTGTTCTTTGTGTTCAGTGGTCA | 60 | |
| SWITCHGRASS | GGTTCTCCAATTCACAAATTTGAGGGCCATAAAGCTGCTGTTCTTTGTGTCCAGTGGTCA | 60 | |
| MAIZE-1 | GGTTCTCCAATTCACAAATTTGAAGGCCATAAAGCTGCTGTTCTTTGTGTTCAGTGGTCA | 60 | |
| MAIZE-2 | GGTTCTCCAATTCACAAATTTGAGGGCCATAAAGCTGCTGTTCTTTGTGTTCAGTGGTCA | 60 | |
| PISUM-SATIVUM | GGGTCTCCTATTCATAAATTTGAGGCTCATAAAGCTGCCGTTCTTTGTGTTCAGTGGTCT | 60 | |
| MEDICAGO-1 | GGGTCTCCTATCCACAAATTTGAGGCTCATAAGGCTGCTGTCCTTTGTGTTCAGTGGTCT | 60 | |
| SOYBEAN-1 | GGGTCACCCATCCATAAATTTGAGGGTCACAAAGCTGCTGTTCTTTGTGTTCAGTGGTCT | 60 | |
| COTTON | GGATCACCCATTTACAAGTTTGAGGGTCATAAAGCTGCTGTTCTATGCGTGCAGTGGTCT | 60 | |
| POPLAR | GGTTTGCCAGTCTATAAATTTGAGGGTCACAAAGCTGCTGTTCTCTGTGTACAGTGGTCT | 60 | |
| PETUNIA | GGCTCACCAGTCCATATCTTTGAAAATCACAAAGCAGCAGTTCTTTGTGTGCAGTGGTGT | 60 | |
| ARABIDOPSIS | GGTTCGCCTATTTACAAATTTGAGGGACACAAAGCTGCTGTTCTTTGTGTTCAGTGGTCT | 60 | |
| CANOLA | GGTACGCCTATTTACAAATTTGAAGGCCACAAAGCTGCTGTTCTTTGCGTTCAGTGGTCT | 60 | |
| MEDICAGO-2 | GGGTCTCCTGTTTATAAATTTGAAGGCCATGATGAACCAGTCCTCTGTGTACAGTGGAAT | 60 | |
| GRAPE | GGATCACCAATCCATACATTTGAGGGCCATACTGCTGCTGTCCTTTGTGTACAGTGGTCT | 60 | |
| RICE-1 | CCTGACAAGGCATCTGTATTTGGAAGCTCTGCGGAAGACGGCTTCTTAAATGTGTGGGAT | 120 | SEQ ID NO:305 |
| RICE-2 | CCTGACAAGGCATCTGTATTTGGAAGCTCTGCGGAAGACGGTTTCTTAAATGTGTGGGAT | 66 | SEQ ID NO:306 |
| SORGHUM | CCTGACAGAGCATCTGTTTTTGGAAGTTCTGCGGAAGATGGTTTCTTAAACGTGTGGGAT | 120 | SEQ ID NO:307 |
| SWITCHGRASS | CCTGACAGAGCATCTGTTTTCGGAAGTTCTGCGGAAGATGGTTTCTTAAATGTGTGGGAT | 120 | SEQ ID NO:308 |
| MAIZE-1 | CCTGACAGAGCATCTGTTTTTGGAAGTTCTGCAGAAGATGGTTTCTTAAACGTGTGGGAT | 120 | SEQ ID NO:309 |
| MAIZE-2 | CCTGACAGAGCATCTGTTTTTGGAAGTTCTGCAGAAGATGGTTTCTTAAATGTTTGGGAC | 120 | SEQ ID NO:310 |
| PISUM-SATIVUM | CCAGACAAATCATCTGTATTTGGAAGTTCAGCAGAAGACGGTCTCTTAAACATTTGGGAT | 120 | SEQ ID NO:311 |
| MEDICAGO-1 | CCAGACAAATCATCTGTATTTGGAAGTTCAGCAGAAGATGGTCTCCTGAACATTTGGGAT | 120 | SEQ ID NO:312 |
| SOYBEAN-1 | CCAGACAAATCATCTGTATTTGGAAGTTCAGCTGAAGATGGTCTCTTAAACATTTGGGAC | 120 | SEQ ID NO:313 |
| COTTON | CCAGACAAATCATCTGTATTTGGCAGTTCTGCTGAGGATGGGCTCTTGAACATTTGGGAC | 120 | SEQ ID NO:314 |
| POPLAR | CCAGATAAGGCATCTGTTTTTGGGAGTTCTGCGGAGGATGGTCTCTTGAATATTTGGGAT | 120 | SEQ ID NO:315 |
| PETUNIA | CCAGACAGGTCCTCTGTATTTGGGAGTACTGCAGAGGATGGTCGTTTGAATATTTGGGAT | 120 | SEQ ID NO:316 |
| ARABIDOPSIS | CCTGATAAGTCATCCGTCTTTGGGAGCTCTGCAGAAGATGGTCTCTTGAACATCTGGGAT | 120 | SEQ ID NO:317 |
| CANOLA | CCTGATAAGTCATCTGTTTTTGGGAGTTCCGCGGAAGATGGTCTCTTGAACATCTGGGAT | 120 | SEQ ID NO:318 |
| MEDICAGO-2 | CCTGCTAAATCATCTGTATTTGGAAGTGGTGCCGAAGATGGAATTATAAACATCTGGGAC | 120 | SEQ ID NO:319 |
| GRAPE | CCGGACAAGGCTTCGATCTTTGGGAGTTCTGCAGAAGATGGTATCTTAAACCTCTGGAAT | 120 | SEQ ID NO:320 |

### Exemplar WD40-6 D Motifs

| | | | |
|---|---|---|---|
| MEDICAGO-2 | SPGLFFRHAGHRDKVVDFHWNASDPWTTVSVSDDCASTGGGGTLQTWR | 48 | SEQ ID NO:321 |
| GRAPE | PPGLFFRHAGHRDKVVDFHWNASDPWTIVSVSDDGESTGGGGTLQIWR | 48 | SEQ ID NO:322 |
| SOYBEAN-1 | PPGLFFQHAGHRDKVVDFHWNAYDPWTIVSVSDDCESTGGGGTLQIWR | 48 | SEQ ID NO:323 |
| PISUM-SATIVUM | PPGLFFQHAGHRDKVVDFHWNAYDPWTIVSVSDDCESTGGGGTLQIWR | 48 | SEQ ID NO:324 |
| MEDICAGO-1 | PPGLFFQHAGHRDKVVDFHWNAHDPWTLVSVSDDCESTGGGGTLQIWR | 48 | SEQ ID NO:325 |
| COTTON | SAGLFFQHAGHRDKVVDFHWNAFDPWTVVSVFDDCETTGGGGTLQIWG | 48 | SEQ ID NO:326 |
| POPLAR | PAGLFFQHAGHRDKVVDFHWNASDPWTVVSVSDDCDTTGGGGTLQIWR | 48 | SEQ ID NO:327 |
| ARABIDOPSIS | PAGLFFQHAGHRDKVVDFHWNASDPWTIVSVSDDCETTGGGGTLQIWR | 48 | SEQ ID NO:328 |
| CANOLA | PAGLFFQHAGHRDKVVDFHWNAEDPWTIVSVSDDCETTGGGGTLQIWR | 48 | SEQ ID NO:329 |
| SWITCHGRASS | PAGLFFQHAGHRDKIVDFHWNSSDPWTIVSVSDDGESTGGGGTLQIWR | 48 | SEQ ID NO:330 |
| RICE-2 | PAGLFFQHAGHRDKIVDFHWNSSDPWTIVSVSDDGESTGGGGTLQIWR | 48 | SEQ ID NO:331 |
| RICE-1 | PAGLFFQHAGHRDKIVDFHWNSSDPWTIVSVSDDGESTGGGGTLQIWR | 48 | SEQ ID NO:332 |
| SORGHUM | PAGLFFQHAGHRDKIVDFHWNSSDPWTIVSVSDDGESTGGGGTLQIWR | 48 | SEQ ID NO:333 |
| MAIZE-2 | PAGLFFQHAGHRDKIVDFHWNSSDPWTIVSVSDDGESTGGGGTLQIWR | 48 | SEQ ID NO:334 |
| MAIZE-1 | PAGLFFQHAGHRDKIVDFHWNSSDPWTIVSVSDDGESTGGGGTLQIWR | 48 | SEQ ID NO:335 |
| PETUNIA | APGLFFQHAGHRDKIVDFHWNVADPWTIVSVSDDCDSTGGGGTLQIWR | 48 | SEQ ID NO:336 |
| PISUM-SATIVUM | CCTCCAGGGTTGTTTTTCCAACATGCTGGTCATAGAGACAAAGTTGTTGACTTCCATTGG | 60 | |
| MEDICAGO-1 | CCTCCAGGGTTGTTTTTCCAACATGCTGGTCATAGAGACAAAGTTGTTGACTTTCACTGG | 60 | |
| SOYBEAN-1 | CCTCCAGGGTTGTTTTTTCAACATGCAGGTCATAGGGATAAAGTTGTTGACTTCCATTGG | 60 | |
| COTTON | TCTGCGGGACTGTTTTTCCAGCACGCTGGACACAGGGACAAAGTTGTTGACTTCCATTGG | 60 | |
| POPLAR | CCTGCAGGATTGTTTTTCCAGCATGCTGGGCACAGGGATAAAGTTGTTGATTTCCATTGG | 60 | |
| MEDICAGO-2 | TCCCCTGGTTTATTCTTTCGTCATGCAGGGCATAGGGATAAGGTTGTTGACTTTCATTGG | 60 | |
| GRAPE | CCTCCAGGTTTATTCTTCAGACATGCTGGCCATAGGGATAAGGTCGTGGACTTCCATTGG | 60 | |
| PETUNIA | GCTCCAGGTTTATTTTTCCAGCATGCTGGGCACAGGGATAAAATTGTTGACTTCCACTGG | 60 | |
| MAIZE-1 | CCAGCCGGGCTTTTCTTTCAGCACGCTGGTCATAGGGATAAGATTGTAGACTTCCACTGG | 60 | |
| MAIZE-2 | CCAGCTGGGCTTTTCTTTCAGCATGCTGGTCATAGGGATAAGATCGTAGACTTCCACTGG | 60 | |
| SORGHUM | CCAGCTGGGCTTTTCTTCCAGCATGCTGGTCATAGGGATAAGATTGTAGACTTCCACTGG | 60 | |
| SWITCHGRASS | CCAGCTGGGCTTTTCTTTCAACATGCTGGTCACAGGGATAAGATTGTAGACTTCCACTGG | 60 | |
| RICE-1 | CCTGCTGGGCTTTTCTTTCAACATGCTGGTCATAGGGATAAGATTGTAGACTTCCACTGG | 60 | |
| RICE-2 | CCTGCTGGGCTTTTCTTTCAACATGCTGGTCATAGGGATAAGATTGTAGACTTCCACTGG | 60 | |
| ARABIDOPSIS | CCCGCTGGGCTCTTCTTCCAGCATGCTGGTCACAGGGACAAAGTTGTTGATTTCCACTGG | 60 | |
| CANOLA | CCGGCTGGTCTCTTCTTCCAGCATGCTGGTCACAGGGACAAAGTTGTTGATTTCCACTGG | 60 | |
| PISUM-SATIVUM | AATGCTTATGATCCATGGACAATTGTAAGTGTGTCTGATGATTGTGAAAGTACTGGTGGA | 120 | SEQ ID NO:337 |
| MEDICAGO-1 | AATGCACATGATCCATGGACACTTGTTAGTGTGTCTGATGATTGCGAAAGTACTGGTGGA | 120 | SEQ ID NO:338 |
| SOYBEAN-1 | AATGCATATGATCCATGGACGATTGTTAGTGTGTCTGATGACTGTGAAAGTACTGGAGGA | 120 | SEQ ID NO:339 |
| COTTON | AATGCATTTGATCCATGGACTGTTGTTAGTGTGTTTGATGACTGTGAAACAACTGGTGGA | 120 | SEQ ID NO:340 |
| POPLAR | AATGCATCTGATCCTTGGACGGTGGTTAGTGTCTCTGATGACTGTGATACCACTGGCGGG | 120 | SEQ ID NO:341 |
| MEDICAGO-2 | AATGCATCTGATCCATGGACAATTGTTAGTGTATCTGATGATTGTGCAAGCACTGGTGGA | 120 | SEQ ID NO:342 |
| GRAPE | AATGCATCGGATCCATGGACAATCGTTAGCGTATCTGATGATGGTGAAAGTACTGGTGGA | 120 | SEQ ID NO:343 |
| PETUNIA | AATGTGGCTGATCCATGGACAATTGTAAGTGTATCTGATGACTGTGACTCCACAGGTGGA | 120 | SEQ ID NO:344 |
| MAIZE-1 | AATTCGTCAGATCCTTGGACAATTGTCAGTGTCTCTGATGATGGCGAGAGCACTGGTGGA | 120 | SEQ ID NO:345 |
| MAIZE-2 | AATTCGTCAGATCCTTGGACAATTGTCAGTGTCTCAGATGATGGTGAGAGCACTGGTGGA | 120 | SEQ ID NO:346 |
| SORGHUM | AATTCGTCAGATCCTTGGACAATTGTCAGTGTATCTGATGATGGTGAGAGCACTGGTGGA | 120 | SEQ ID NO:347 |
| SWITCHGRASS | AATTCATCGGATCCTTGGACAATCGTCAGTGTCTCAGATGATGGTGAGAGCACCGGTGGA | 120 | SEQ ID NO:348 |
| RICE-1 | AATTCTTCGGATCCTTGGACTATTGTGAGTGTGTCTGATGATGGTGAGAGTACTGGTGGA | 120 | SEQ ID NO:349 |
| RICE-2 | AATTCTTCGGATCCTTGGACTATTGTGAGTGTGTCTGATGATGGTGAGAGTACTGGTGGA | 120 | SEQ ID NO:350 |
| ARABIDOPSIS | AATGCTTCAGACCCTTGGACTATTGTCAGTGTTTCTGATGACTGTGAGACTACTGGTGGA | 120 | SEQ ID NO:351 |
| CANOLA | AATGCAGAGGACCCTTGGACTATTGTCAGTGTTTCTGATGACTGCGAGACTACTGGTGGA | 120 | SEQ ID NO:352 |

### Example 13

### Functional confirmation of FVE homologues and useful motifs

Expression vectors comprising a candidate sequence are introduced into Arabidopsis and assessed for biomass production, stem diameter, delayed flowering and other physiological phenotypes associated with the FVE mutant. Sequences disclosed as SEQ ID NO: 1, 3, 5, 6, 7, 9, 11, 13, 15, 17, 19, 21, 23, 25, 27, 29, 31, 33, 35, 206, 216, 220, 239, and 241 or a portion thereof, such as SEQ ID NO:51-64, 245, 246, 79-92, 233, 248, 107-120, 234, 252, 135-148, 235, 250, 163-176, 256, 254, 273-288, 305-320 and 337-352 or an expression cassette such as SEQ ID NO:372-379, are expressed in Arabidopsis plants, and biomass productivity assessed as described herein. Optionally, the expression of FVE genes can be evaluated in any transformable species, for example, Brassica, maize, cotton, soybean or rice.

1) A method of producing a plant having increased biomass accumulation relative to a wild-type plant, the method comprising introducing into a plant cell a nucleic acid construct that inhibits the expression or activity of FVE.

2) The plant produced by the method of claim 1.

3) The method of claim 1 wherein the method comprises the steps of:
a) providing a nucleic acid construct comprising a promoter operably linked to a nucleic acid construct that inhibits FVE activity;
b) inserting said nucleic construct into a vector;
c) transforming a plant, tissue culture, or a plant cell with the vector to obtain a plant, tissue culture or a plant cell with decreased FVE activity;
d) growing said plant or regenerating a plant from said tissue culture or plant cell,
wherein a plant having increased biomass accumulation relative to a wild-type plant is produced.

4) The transgenic plant of claim 3 wherein the nucleic acid construct comprises an FVE-S1 motif.

5) The transgenic plant of claim 3 wherein the nucleic acid construct comprises an FVE-S2 motif.

6) The transgenic plant of claim 3 wherein the nucleic acid construct comprises an FVE-WD40 motif.

7) The transgenic plant of claim 3 wherein the nucleic acid construct comprises an FVE sequence of at least 21 nucleotides in length or its compliment.

8) A transgenic seed produced by the transgenic plant of claim 1 or claim 3, 4, 5 or 6 wherein said seed produces plant having increased biomass accumulation relative to a wild-type plant.

9) A plant having a non-naturally occurring mutation in an FVE gene, wherein said plant has decreased FVE expression or activity and said plant has increased biomass relative to a wild-type control.

10) A nucleic acid comprising the nucleotide sequence selected from the group consisting of SEQ ID NO's:1, 5, 206, 216, 220, 239, 241, 368, 371, 372, 373, 374, 375, 376, 377, 378, and 379.
